(19) United States
(12) Patent Application Publication
(10) Pub. No.: US 2008/0280968 A1
(43) Pub. Date: Nov. 13, 2008

(54) METHODS OF USING [3.2.0] HETEROCYCLIC COMPOUNDS AND ANALOGS THEREOF FOR TREATING INFECTIOUS DISEASES

(75) Inventor: Michael Palladino, Olivenhain, CA (US)

Correspondence Address: KNOBBE MARTENS OLSON & BEAR LLP, 2040 MAIN STREET, FOURTEENTH FLOOR, IRVINE, CA, 92614, US

(73) Assignee: NEREUS PHARMACEUTICALS, INC., San Diego, CA (US)

(21) Appl. No.: 12/114,449

(22) Filed: May 2, 2008

Related U.S. Application Data
(60) Provisional application No. 60/916,243, filed on May 4, 2007.

(51) Int. Cl. A61K 31/4152; A61K 31/4965; A61K 31/44; A61K 31/496; A61K 31/13; A61K 31/7036; A61K 31/70; A61P 31/04

(52) U.S. Cl. 514/421; 514/255.06; 514/354; 514/254.11; 514/37; 514/34

(57) ABSTRACT

Disclosed are methods of treating infectious diseases comprising administering to the animal, a therapeutically effective amount of a heterocyclic compound. The animal is a mammal, preferably a human or a rodent.

### CROSS-REFERENCE TO RELATED APPLICATIONS

This application claims the benefit of U.S. Provisional Application No. 60/916,243, filed May 4, 2007, which is incorporated herein by reference in its entirety.

### BACKGROUND OF THE INVENTION

1. Field of the Invention

The present invention relates to certain compounds and to methods for the preparation and the use of certain compounds in the fields of chemistry and medicine. Embodiments of the invention disclosed herein relate to methods of using heterocyclic compounds. In some embodiments, the compounds are used as proteasome inhibitors. In other embodiments, the compounds are used to treat infectious diseases.

2. Description of the Related Art

Infectious diseases caused, for example, by bacteria, fungi and protozoa are becoming increasingly difficult to treat and cure. For example, more and more bacteria, fungi and protozoa are developing resistance to current antibiotics and chemotherapeutic agents. A stark example of the growing problem of drug resistant infections can be seen in the case of Tuberculosis.

About two billion people are thought to be infected with the bacillus Mycobacterium tuberculosis (“Mtb”), the causative agent of tuberculosis (“TB”). The majority of those infected do not show signs of disease; however, each year about 8 million individuals develop active tuberculosis and about 2 million die (Dye et al., “Consensus Statement. Global Burden of Tuberculosis: Estimated Incidence, Prevalence, and Mortality by Country. WHO Global Surveillance and Monitoring Project,” JAMA 282(7):677-86 (1999)). Cure of tuberculosis requires months of treatment with multiple anti-infective agents. Incomplete treatment is common and encourages the emergence of multi-drug resistant (“MDR”) strains. MDR isolates are detected in all nations and prevalent in some. Infection can be acquired by sharing airspace with an individual with cavitary disease, with an infectious dose estimated at 1-10 inhaled bacilli.

Mtb infection can persist for decades (World Health Organization, “Tuberculosis and AIDS: Statement on AIDS and Tuberculosis,” Bull. Int. Tuberc. Lung Dis. 64:88111 (1989); Bloom et al., “Tuberculosis: Commentary on a Re-Emergent Killer,” Science 257:55-64 (1992); Russell, “Mycobacterium Tuberculosis: Here Today, and Here Tomorrow,” Nat. Rev. Mol. Cell. Biol. 2:1-9 (2001); Raupach et al., “Immune Responses to Intracellular Bacteria,” Curr. Opin. Imm. 13:417-428 (2001)). The normal immune system creates an environment in which Mtb is not completely sterilized, yet replicates so little that 90% of immune-competent hosts who are infected with Mtb never develop overt TB. During latent infection, the primary residence of Mtb is the macrophage.

Among the most successful forms of anti-Mtb chemotherapy is that applied naturally by the host. Of these, nitric oxide (“NO”) is the only molecule known to be produced by mammalian cells that can kill tubercle bacilli in vitro with a potency (−150 nM) comparable to that of chemotherapy. That the primary product of iNOS is mycobacteriacidal provides one type of evidence consistent with a role for iNOS in controlling tuberculosis.

With the emergence of drug resistant strains of bacteria, such as Tubercle Bacillus, a need exists for additional anti-microbial agents, to treat infectious diseases. Multi-drug resistant TB is defined as resistance to the two most effective first line TB drugs: rifampicin and isoniazid. Extensively drug-resistant TB is also resistant to three or more of the six classes of second-line drugs. Over one-third of the world's population now has the Tubercle Bacillus bacterium (TB) in their bodies and new infections are occurring at a rate of one per second according to the World Health Organization (WHO). In light that an increasing amount of TB is becoming drug resistant, new anti-microbial agents are of interest to treat this disease. A continuing effort is being made by individual investigators, academia and companies to identify new, potentially useful anti-microbial agents.

Marine-derived natural products are a rich source of potential new anti-microbial agents. The oceans are massively complex and house a diverse assemblage of microbes that occur in environments of extreme variations in pressure, salinity, and temperature. Marine microorganisms have therefore developed unique metabolic and physiological capabilities that not only ensure survival in extreme and varied habitats, but also offer the potential to produce metabolites that would not be observed from terrestrial microorganisms (Okami, Y. 1993 J Mar Biotechnol 1:59). Representative structural classes of such metabolites include terpenes, peptides, polyketides, and compounds with mixed biosynthetic origins. Many of these molecules have demonstrable anti-tumor, anti-bacterial, anti-fungal, anti-inflammatory or immunosuppressive activities (Bull, A. T. et al. 2000 Microbiol Mol Biol Rev 64:573; Cragg, G. M. & D. J. Newman 2002 Trends Pharmacol Sci 23:404; Kerr, R. G. & S. S. Kerr 1999 Exp Opin Ther Patents 9:1207; Moore, B. S 1999 Nat Prod Rep 16:653; Faulkner, D. J. 2001 Nat Prod Rep 18:1; Mayer, A. M. & V. K. Lehmann 2001 Anticancer Res 21:2489), validating the utility of this source for isolating invaluable therapeutic agents. Further, the isolation of novel anti-microbial agents that represent alternative mechanistic classes to those currently on the market will help to address resistance concerns, including any mechanism-based resistance that may have been engineered into pathogens for bioterrorism purposes. Additionally, anti-microbial agents that enhance the host organisms natural defenses are thought to be of particular interest.

### SUMMARY OF THE INVENTION

The embodiments disclosed herein generally relate to chemical compounds, including heterocyclic compounds and analogs thereof. Some embodiments are directed to the use of compounds as proteasome inhibitors.

In certain embodiments, the compounds are used to treat infectious diseases. The infectious agent can be a microbe, for example, bacteria, fungi, protozoans, and microscopic algae, or viruses. In some embodiments, the infectious agent can be Tubercle Bacillus (Tuberculosis abbreviated as TB). For example, Tuberculosis is caused by mycobacteria, primarily Mycobacterium tuberculosis. Additionally, other mycobacteria such as Mycobacterium bovis, Mycobacterium africanum and Mycobacterium microti can also cause tuberculosis, but these species do not usually infect healthy adults. In some embodiments the infectious agent is a parasite. Certain embodiments relate to methods of treating infectious agents in animals. The method can include, for example, administering an effective amount of a compound to a patient in need thereof. Other embodiments relate to the use of compounds in the manufacture of a pharmaceutical or medicament for the treatment of infectious diseases.

Some embodiments relate to uses of a compound having the structure of any one of Formulas I and II, and pharmaceutically acceptable salts and pro-drugs thereof:

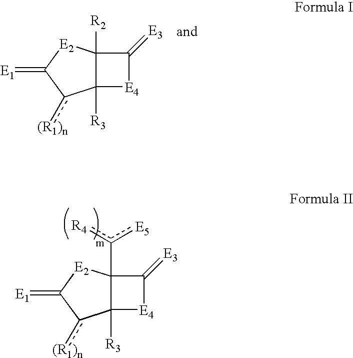

wherein:

the dashed lines represent a single or a double bond;

each R₁ is separately a hydrogen, a halogen, a cyano, a nitro, an azido, a hydroxy, or a thiocyano, or selected from the group consisting of optionally substituted: C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl;

n is 1 or 2, where if n is 2, then each R₁ can be the same or different;

m is 1 or 2, where if m is 2, then each R₄ can be the same or different;

R₂ is a hydrogen, a halogen, a cyano, a nitro, an azido, a hydroxy, or a thiocyano, or selected from the group consisting of optionally substituted: C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl;

R₃ is a halogen or selected from the group consisting of optionally substituted C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, nitro, azido, phenyl, cycloalkylacyl, hydroxy, alkylthio, arylthio, oxysulfonyl, carboxy, cyano, and halogenated alkyl including polyhalogenated alkyl;

each of E₁, E₃, E₄ and E₅ is an optionally substituted heteroatom;

E₂ is an optionally substituted heteroatom or —CH₂— group; and

each R₄ is separately a halogen, a cyano, a nitro, an azido, or a thiocyano, or selected from the group consisting of optionally substituted C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, hydroxy, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl.

In some embodiments, preferably R₁ can be a substituted or unsubstituted C₁ to C₅ alkyl. For example, methyl, ethyl, propyl, isopropyl, butyl, isobutyl, tert-butyl, and pentyl are preferred. In some embodiments, R₁ is not a substituted or unsubstituted, unbranched C₆ alkyl.

In some embodiments a compound having the structure of any one of Formulas I and II, and pharmaceutically acceptable salts and pro-drugs thereof can be used to treat an infectious disease. For example, the infectious disease can be selected from the group consisting of Bacteremia, Botulism, Brucellosis, Clostridium Difficile, Campylobacter Infection, Cat Scratch Disease, Chancroid, Chlamydia, Cholera, Clostridium Perfringens, Bacterial Conjunctivitis, Diphtheria, E. Coli Infections, Ehrlichiosis, Epididymitis, Gardnerella, Gas Gangrene, Gonorrhea, Helicobacter Pylori, Haemophilus, Influenzae B, Impetigo, Intertrigo, Leprosy, Listeriosis, Lyme Disease, Methicillin Resistant Staphylococcus Aureus, Orchitis, Osteomyelitis, Otitis, Media Pertussis, Plague, Pneumonia, Prostatitis Pyelonephritis, Q Fever, Rocky Mountain Spotted Fever, Salmonellosis, Scarlet Fever, Sepsis, Shigellosis, Staphylococcal Infections, Streptococcal Infections, Syphilis, Tetanus, Toxic Shock Syndrome, Trachoma, Traveller's Diarrhea, Tuberculosis, Tularemia, Typhoid Fever, Typhus Fever, Urinary Tract Infections, Bacterial Vaginosis, Pertussis, Yersiniosis, malaria, African trypanosomiasis, candidiasis, histoplasmosis, blastomycosis, coccidioidomycosis, aspergillisis, and mucormycosis and the like. In a preferred embodiment, the infectious disease is a bacterial infectious disease. For example, the bacterial infectious disease can be selected from the group consisting of Bacteremia, Botulism, Brucellosis, Clostridium Difficile, Campylobacter Infection, Cat Scratch Disease, Chancroid, Chlamydia, Cholera, Clostridium Perfringens, Bacterial Conjunctivitis, Diphtheria, E. Coli Infections, Ehrlichiosis, Epididymitis, Gardnerella, Gas Gangrene, Gonorrhea, Helicobacter Pylori, Haemophilus, Influenzae B, Impetigo, Intertrigo, Leprosy, Listeriosis, Lyme Disease, Methicillin Resistant Staphylococcus Aureus, Orchitis, Osteomyelitis, Otitis, Media Pertussis, Plague, Pneumonia, Prostatitis Pyelonephritis, Q Fever, Rocky Mountain Spotted Fever, Salmonellosis, Scarlet Fever, Sepsis, Shigellosis, Staphylococcal Infections, Streptococcal Infections, Syphilis, Tetanus, Toxic Shock Syndrome, Trachoma, Traveller's Diarrhea, Tuberculosis, Tularemia, Typhoid Fever, Typhus Fever, Urinary Tract Infections, Bacterial Vaginosis, Pertussis, Yersiniosis, and the like.

In some embodiments, the compound used to treat the bacterial infectious disease can be Salinosporamide A. In a preferred embodiment, Salinosporamide A can be used to treat Tuberculosis. Further embodiments relate to treating Tuberculosis with Salinosporamide A in combination with one or more anti-microbial agents. For example, the anti-microbial agent or agents can be selected form the group consisting of isoniazid, rifampin, ethambutol, pyrazinamide, rifater, streptomycin, rifapentine, epoxomicin and the like. In some embodiments, Salinosporamide A used in conjunction with other anti-microbial agents can prevent Mycobacterium Tuberculosis from becoming multi-drug resistant. In some embodiments, Salinosporamide A can be used prior to treatment with other anti-microbial agents. In a preferred embodiment, Salinosporamide A can be used prior to treatment of Tuberculosis with other anti-microbial agents preventing Mycobacterium Tuberculosis from becoming multi-drug resistant. In some embodiments, Salinosporamide A can be used to treat multi-drug resistant Tuberculosis. In some embodiments, Salinosporamide A can be used to treat Tuberculosis in which Mycobacterium Tuberculosis has become drug resistant.

Further embodiments relate to pharmaceutical compositions which include a compound of a formula selected from Formulae I and II. The pharmaceutical compositions can further include an anti-microbial agent.

Other embodiments relate to methods of inhibiting proteasome activity that include the step contacting a cell with a compound of a formula selected from Formula I and II, and pharmaceutically acceptable salts and pro-drugs thereof. In one embodiment, a compound of a formula selected from Formula I and II, and pharmaceutically acceptable salts and pro-drugs thereof, can inhibit proteasomes in Mycobacterium Tuberculosis cells. In some embodiments, a compound of a formula selected from Formula I and II, and pharmaceutically acceptable salts and pro-drugs thereof, can selectively inhibit proteasomes in Mycobacterium Tuberculosis cells while not inhibiting or inhibiting less proteasome activity in other cells. In a preferred embodiment, Salinosporamide A can inhibit proteasomes in Mycobacterium Tuberculosis cells. In a further preferred embodiment, Salinosporamide A can selectively inhibit proteasomes in Mycobacterium Tuberculosis cells while not inhibiting or inhibiting less proteasome activity in other cells.

Some embodiments relate to methods for treating a microbial illness including administering an effective amount of a compound of a formula selected from Formulae I and II to a patient in need thereof.

Some embodiments relate to methods for treating a microbial illness including administering an effective amount of a compound of a formula selected from Formulae I and II to a patient in need thereof.

### BRIEF DESCRIPTION OF THE DRAWINGS

The accompanying drawings, which are incorporated in and form part of the specification, merely illustrate certain preferred embodiments of the present invention. Together with the remainder of the specification, they are meant to serve to explain preferred modes of making certain compounds of the invention to those of skilled in the art. In the drawings:

FIG. 1 shows inhibition of the chymotrypsin-like activity of rabbit muscle proteasomes.

FIG. 2 shows inhibition of the PGPH and Caspase-like activity of rabbit muscle proteasomes.

FIG. 3 shows inhibition of the chymotrypsin-like activity of human erythrocyte proteasomes.

FIG. 4 shows proteasomal activity in PWBL prepared from Salinosporamide A (Formula II-16) treated mice.

FIG. 5 shows epoxomicin treatment in the PWBL assay.

FIG. 6 shows intra-assay comparison.

### DETAILED DESCRIPTION OF THE PREFERRED EMBODIMENT

Numerous references are cited herein. The references cited herein, including the U.S. patents cited herein, are each to be considered incorporated by reference in their entirety into this specification.

Embodiments of the invention include, but are not limited to, providing a method for the preparation of compounds, including compounds, for example, those described herein and analogs thereof, and to providing a method for producing pharmaceutically acceptable anti-microbial compositions, for example. The methods can include the compositions in relatively high yield, wherein the compounds and/or their derivatives are among the active ingredients in these compositions. Other embodiments relate to providing novel compounds not obtainable by currently available methods. Furthermore, embodiments relate to methods of treating infectious diseases, particularly those affecting humans. In some embodiments, one or more formulae, one or more compounds, or groups of compounds can be specifically excluded from use in any one or more of the methods of treating the conditions described herein. The methods may include, for example, the step of administering an effective amount of a member of a class of new compounds. Preferred embodiments relate to the compounds and methods of making and using such compounds disclosed herein, but not necessarily in all embodiments of the present invention, these objectives are met.

For the compounds described herein, each stereogenic carbon can be of R or S configuration. Although the specific compounds exemplified in this application can be depicted in a particular configuration, compounds having either the opposite stereochemistry at any given chiral center or mixtures thereof are also envisioned. When chiral centers are found in the derivatives of this invention, it is to be understood that the compounds encompasses all possible stereoisomers.

Some embodiments relate to uses of a compound having the structure of any one of Formulas I and II, and pharmaceutically acceptable salts and pro-drugs thereof:

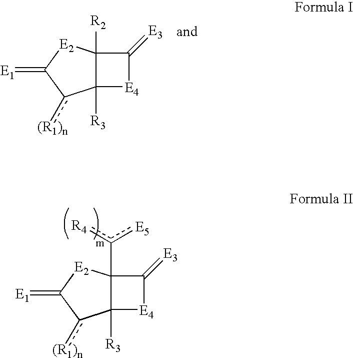

wherein:

the dashed lines represent a single or a double bond;

each R₁ is separately a hydrogen, a halogen, a cyano, a nitro, an azido, a hydroxy, or a thiocyano, or selected from the group consisting of optionally substituted: C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl;

n is 1 or 2, where if n is 2, then each R₁ can be the same or different;

m is 1 or 2, where if m is 2, then each R₄ can be the same or different;

R₂ is a hydrogen, a halogen, a cyano, a nitro, an azido, a hydroxy, or a thiocyano, or selected from the group consisting of optionally substituted: C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl;

R₃ is a halogen or selected from the group consisting of optionally substituted C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, nitro, azido, phenyl, cycloalkylacyl, hydroxy, alkylthio, arylthio, oxysulfonyl, carboxy, cyano, and halogenated alkyl including polyhalogenated alkyl;

each of E₁, E₃, E₄ and E₅ is an optionally substituted heteroatom;

E₂ is an optionally substituted heteroatom or —CH₂— group; and

each R₄ is separately a halogen, a cyano, a nitro, an azido, or a thiocyano, or selected from the group consisting of optionally substituted C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, hydroxy, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl.

In some embodiments E₅ can be, for example, OH, O, OR₁₀, S, SR₁₁, SO₂R₁₁, NH, NH₂, NOH, NHOH, NR₁₂, and NHOR₁₃, wherein R₁₀₋₁₃ may separately include, for example, hydrogen, a substituted or unsubstituted of any of the following: alkyl, an aryl, a heteroaryl, and the like. R₃ can be methyl. Furthermore, R₄ may include a cyclohexyl. Also, each of E₁, E₃ and E₄ can be O and E₂ can be NH. Preferably, R₁ can be CH₂CH₂X, wherein X is selected from the group consisting of hydrogen, fluorine, chlorine, bromine, and iodine; wherein R₄ may include a cyclohexyl; wherein R₃ can be methyl; and wherein each of E₁, E₃ and E₄ separately can be O and E₂ can be NH. In some embodiments, R₁ can be alkyl optionally substituted with a boranic ester or boranic ester. For example, the boronic ester can be B(OMethyl)₂, B(OEthyl)₂, B(OPropyl)₂, B(OPhenyl)₂, and the like.

In certain embodiments, the compound is Salinosporamide A;

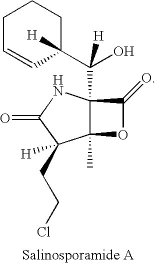

Some embodiments provide a method of treating or preventing infectious diseases comprising administering to an animal a compound having the structure of any one of Formulas I and II, or a pharmaceutically acceptable salt or pro-drug ester thereof:

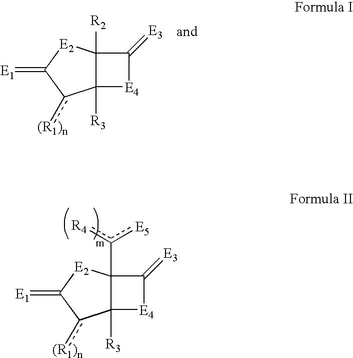

wherein:

the dashed lines represent a single or a double bond;

each R₁ is separately a hydrogen, a halogen, a cyano, a nitro, an azido, a hydroxy, or a thiocyano, or selected from the group consisting of optionally substituted: C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl;

n is 1 or 2, where if n is 2, then each R₁ can be the same or different;

m is 1 or 2, where if m is 2, then each R₄ can be the same or different;

R₂ is a hydrogen, a halogen, a cyano, a nitro, an azido, a hydroxy, or a thiocyano, or selected from the group consisting of optionally substituted: C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl;

R₃ is a halogen or selected from the group consisting of optionally substituted C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, nitro, azido, phenyl, cycloalkylacyl, hydroxy, alkylthio, arylthio, oxysulfonyl, carboxy, cyano, and halogenated alkyl including polyhalogenated alkyl;

each of E₁, E₃, E₄ and E₅ is an optionally substituted heteroatom;

E₂ is an optionally substituted heteroatom or —CH₂— group;

each R₄ is separately a halogen, a cyano, a nitro, an azido, or a thiocyano, or selected from the group consisting of optionally substituted C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, hydroxy, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl; and

wherein the infectious disease is caused by a bacterial infectious disease.

In a preferred embodiment, the animal is a human.

In certain embodiments, the infectious disease can be selected from the group consisting of Bacteremia, Botulism, Brucellosis, Clostridium Difficile, Campylobacter Infection, Cat Scratch Disease, Chancroid, Chlamydia, Cholera, Clostridium Perfringens, Bacterial Conjunctivitis, Diphtheria, E. Coli Infections, Ehrlichiosis, Epididymitis, Gardnerella, Gas Gangrene, Gonorrhea, Helicobacter Pylori, Haemophilus, Influenzae B, Impetigo, Intertrigo, Leprosy, Listeriosis, Lyme Disease, Methicillin Resistant Staphylococcus Aureus, Orchitis, Osteomyelitis, Otitis, Media Pertussis, Plague, Pneumonia, Prostatitis Pyelonephritis, Q Fever, Rocky Mountain Spotted Fever, Salmonellosis, Scarlet Fever, Sepsis, Shigellosis, Staphylococcal Infections, Streptococcal Infections, Syphilis, Tetanus, Toxic Shock Syndrome, Trachoma, Traveller's Diarrhea, Tuberculosis, Tularemia, Typhoid Fever, Typhus Fever, Urinary Tract Infections, Bacterial Vaginosis, Pertussis, Yersiniosis, malaria, African trypanosomiasis, candidiasis, histoplasmosis, blastomycosis, coccidioidomycosis, aspergillisis, and mucormycosis and the like.

In a typical embodiment, the bacterial infection can be Tuberculosis. In some embodiments, the bacteria causing Tuberculosis can be selected from the group consisting of Mycobacterium bovis, Mycobacterium africanum and Mycobacterium microti. For example, the bacteria causing Tuberculosis can be Mycobacterium tuberculosis.

In a typical embodiment, the compound can be Salinosporamide A:

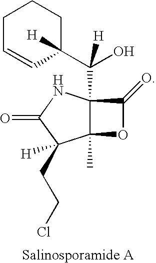

In some embodiments, the method further comprises co-administering one or more anti-infective agent(s). For example, the anti-infective agent(s) can be selected from the group consisting of isoniazid, rifampin, ethambutol, pyrazinamide, rifater, streptomycin, rifapentine, epoxomicin, and the like.

Some embodiments provide a pharmaceutical composition comprising a compound of any one of Formulas I and II:

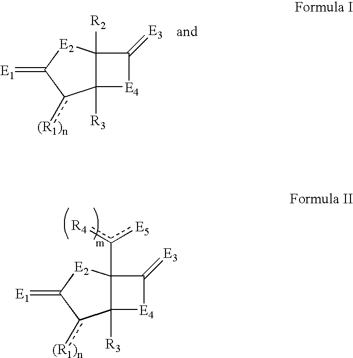

wherein:

the dashed lines represent a single or a double bond;

each R₁ is separately a hydrogen, a halogen, a cyano, a nitro, an azido, a hydroxy, or a thiocyano, or selected from the group consisting of optionally substituted: C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl;

n is 1 or 2, where if n is 2, then each R₁ can be the same or different;

m is 1 or 2, where if m is 2, then each R₄ can be the same or different;

R₂ is a hydrogen, a halogen, a cyano, a nitro, an azido, a hydroxy, or a thiocyano, or selected from the group consisting of optionally substituted: C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl;

R₃ is a halogen or selected from the group consisting of optionally substituted C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, nitro, azido, phenyl, cycloalkylacyl, hydroxy, alkylthio, arylthio, oxysulfonyl, carboxy, cyano, and halogenated alkyl including polyhalogenated alkyl;

each of E₁, E₃, E₄ and E₅ is an optionally substituted heteroatom;

E₂ is an optionally substituted heteroatom or —CH₂— group; and

each R₄ is separately a halogen, a cyano, a nitro, an azido, or a thiocyano, or selected from the group consisting of optionally substituted C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, hydroxy, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl.

In some embodiments E₅ can be, for example, OH, O, OR₁₀, S, SR₁₁, SO₂R₁₁, NH, NH₂, NOH, NHOH, NR₁₂, and NHOR₁₃, wherein R₁₀₋₁₃ may separately include, for example, hydrogen, a substituted or unsubstituted of any of the following: alkyl, an aryl, a heteroaryl, and the like. R₃ can be methyl. Furthermore, R₄ may include a cyclohexyl. Also, each of E₁, E₃ and E₄ can be O and E₂ can be NH. Preferably, R₁ can be CH₂CH₂X, wherein X is selected from the group consisting of hydrogen, fluorine, chlorine, bromine, and iodine; wherein R₄ may include a cyclohexyl; wherein R₃ can be methyl; and wherein each of E₁, E₃ and E₄ separately can be O and E₂ can be NH. In some embodiments, R₁ can be alkyl optionally substituted with a boranic ester or boranic ester. For example, the boronic ester can be B(OMethyl)₂, B(OEthyl)₂, B(OPropyl)₂, B(OPhenyl)₂, and the like.

Some embodiments provide a method of treating infectious diseases comprising administering to an animal a compound having the structure of Formula I:

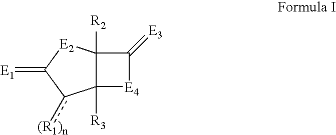

wherein:

the dashed lines represent a single or a double bond;

each R₁ is separately a hydrogen, a halogen, a cyano, a nitro, an azido, a hydroxy, or a thiocyano, or selected from the group consisting of optionally substituted: C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl;

n is 1 or 2, where if n is 2, then each R₁ can be the same or different;

R₂ is a hydrogen, a halogen, a cyano, a nitro, an azido, a hydroxy, or a thiocyano, or selected from the group consisting of optionally substituted: C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl;

R₃ is a halogen or selected from the group consisting of optionally substituted C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, nitro, azido, phenyl, cycloalkylacyl, hydroxy, alkylthio, arylthio, oxysulfonyl, carboxy, cyano, and halogenated alkyl including polyhalogenated alkyl;

each of E₁, E₃, and E₄ is an optionally substituted heteroatom;

E₂ is an optionally substituted heteroatom or —CH₂— group; and

wherein the infectious disease is caused by a bacterial infectious disease.

In a preferred embodiment, the animal is a human.

Some embodiments provide a method of treating infectious diseases comprising administering to an animal a compound having the structure of Formula II:

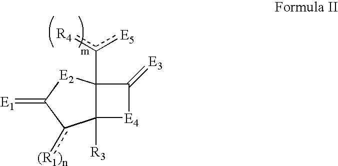

wherein:

the dashed lines represent a single or a double bond;

each R₁ is separately a hydrogen, a halogen, a cyano, a nitro, an azido, a hydroxy, or a thiocyano, or selected from the group consisting of optionally substituted: C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl;

n is 1 or 2, where if n is 2, then each R₁ can be the same or different;

m is 1 or 2, where if m is 2, then each R₄ can be the same or different;

R₃ is a halogen or selected from the group consisting of optionally substituted C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, nitro, azido, phenyl, cycloalkylacyl, hydroxy, alkylthio, arylthio, oxysulfonyl, carboxy, cyano, and halogenated alkyl including polyhalogenated alkyl;

each of E₁, E₃, E₄ and E₅ is an optionally substituted heteroatom;

E₂ is an optionally substituted heteroatom or —CH₂— group;

each R₄ is separately a halogen, a cyano, a nitro, an azido, or a thiocyano, or selected from the group consisting of optionally substituted C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, hydroxy, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl; and

wherein the infectious disease is caused by a bacterial infectious disease.

In a preferred embodiment, the animal is a human.

In some embodiments n can be equal to 1, while in others it can be equal to 2. When n is equal to 2, the substituents can be the same or can be different. Furthermore, in some embodiments R₃ is not a hydrogen. In some embodiments m can be equal to 1 or 2, and when m is equal to 2, R₄ can be the same or different.

In some embodiments E₅ can be, for example, OH, O, OR₁₀, S, SR₁₁, SO₂R₁₁, NH, NH₂, NOH, NHOH, NR₁₂, and NHOR₁₃, wherein R₁₀₋₁₃ may separately include, for example, hydrogen, a substituted or unsubstituted of any of the following: alkyl, an aryl, a heteroaryl, and the like. R₃ can be methyl. Furthermore, R₄ may include a cyclohexyl. Also, each of E₁, E₃ and E₄ can be O and E₂ can be NH. Preferably, R₁ can be CH₂CH₂X, wherein X is selected from the group consisting of hydrogen, fluorine, chlorine, bromine, and iodine; wherein R₄ may include a cyclohexyl; wherein R₃ can be methyl; and wherein each of E₁, E₃ and E₄ separately can be O and E₂ can be NH. In some embodiments, R₁ can be alkyl optionally substituted with a boranic ester or boranic ester. For example, the boronic ester can be B(OMethyl)₂, B(OEthyl)₂, B(OPropyl)₂, B(OPhenyl)₂, and the like.

In some embodiments, R₂ is not cyclohex-2-enyl carbinol when one of the R₁ substituents is ethyl or chloroethyl and R₃ is methyl.

In some embodiments, R₁ can be an optionally substituted C₁ to C₅ alkyl. For example, R₁ can be methyl, ethyl, propyl, isopropyl, butyl, isobutyl, tert-butyl, pentyl and the like. In some embodiments, R₁ is not a substituted or unsubstituted, unbranched C₆ alkyl.

In another embodiment, E₅ can be OH. For example, the compound may have the following Formula I-1:

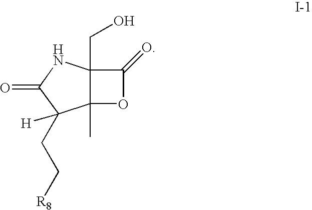

In some embodiments, for example, R₈ can be selected from the group consisting of hydrogen, fluorine, chlorine, bromine and iodine.

As an example, Formula I-1 may have the following stereochemistry:

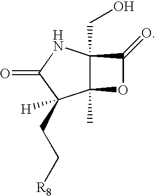

In some embodiments, for example, R₈ can be selected from the group consisting of hydrogen, fluorine, chlorine, bromine and iodine.

Still a further exemplary compound of Formula II is a compound having the following Formula I-2:

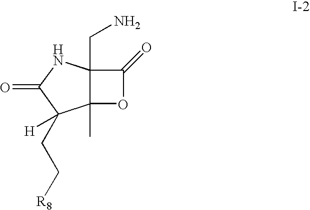

In some embodiments, for example, R₈ can be selected from the group consisting of hydrogen, fluorine, chlorine, bromine and iodine.

For example, Formula I-2 may have the following stereochemistry:

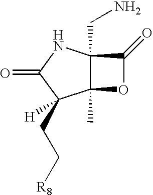

In some embodiments, for example, R₈ can be selected from the group consisting of hydrogen, fluorine, chlorine, bromine and iodine.

An exemplary compound of Formula II can have the following Formula II-1:

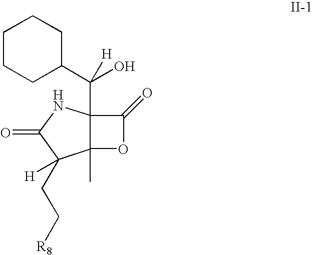

In some embodiments, for example, R₈ can be selected from the group consisting of hydrogen, fluorine, chlorine, bromine and iodine.

Exemplary stereochemistry can be as follows:

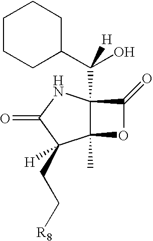

In some embodiments, the compound of Formula I can have any of the following structures of Formulae II-2, II-3, and II-4:

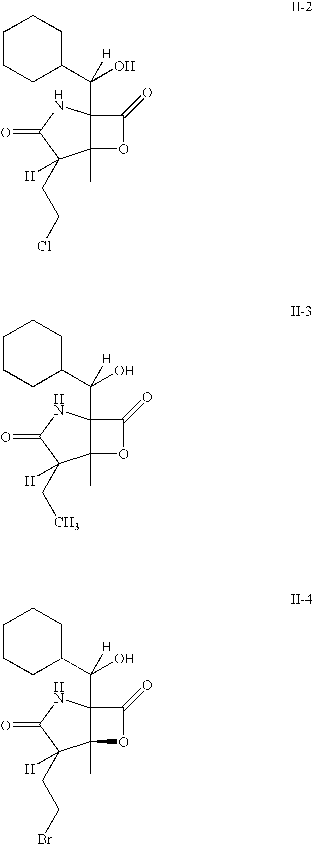

The following is exemplary stereochemistry for compounds having the structures of Formulae II-2, II-3, and II-4, respectively:

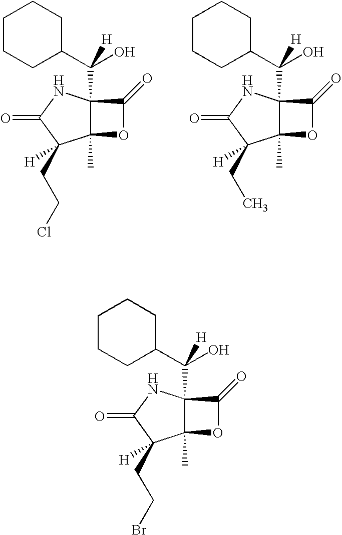

In other embodiments wherein R₄ may include a 7-oxa-bicyclo[4.1.0]hept-2-yl). An exemplary compound of Formula I is the following Formula II-5:

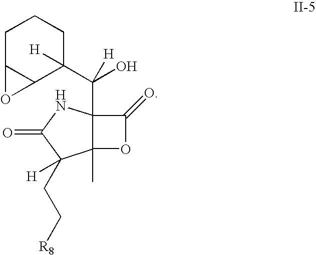

In some embodiments, for example, R₈ can be selected from the group consisting of hydrogen, fluorine, chlorine, bromine and iodine.

The following are examples of compounds of Formula II-5 having the structures of Formulae II-5A and II-5B:

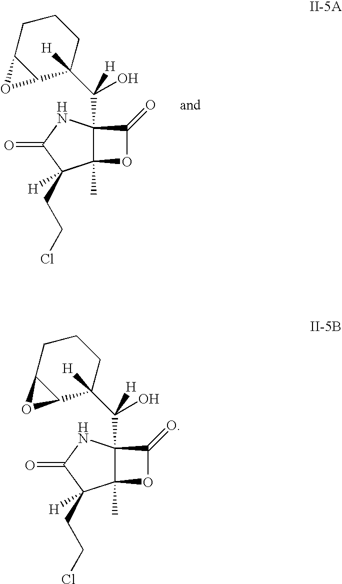

In still further embodiments, at least one R₄ may include an optionally substituted branched alkyl. For example, a compound of Formula I can be the following Formula II-6:

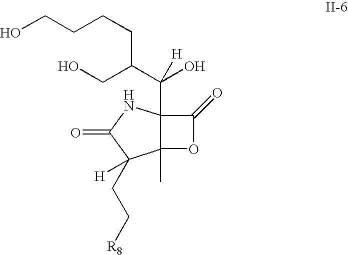

In some embodiments, for example, R₈ can be selected from the group consisting of hydrogen, fluorine, chlorine, bromine and iodine.

The following is exemplary stereochemistry for a compound of Formula II-6:

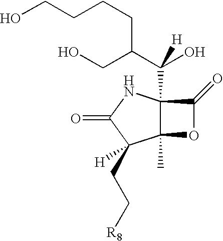

As another example, the compound of Formula I can be the following Formula II-7:

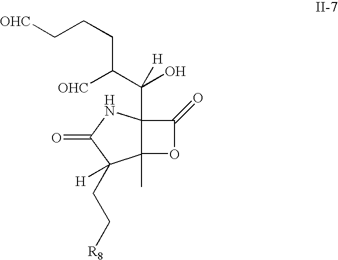

In some embodiments, for example, R₈ can be selected from the group consisting of hydrogen, fluorine, chlorine, bromine and iodine.

The following is exemplary stereochemistry for a compound having the structure of Formula II-7:

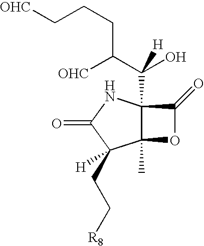

In other embodiments, at least one R₄ can be an optionally substituted cycloalkyl and E₅ can be an oxygen. For example, R₄ can be cyclopropyl, cyclopentyl, cyclohexyl, cycloheptyl, and the like. An exemplary compound of Formula I can have the structure of Formula II-8:

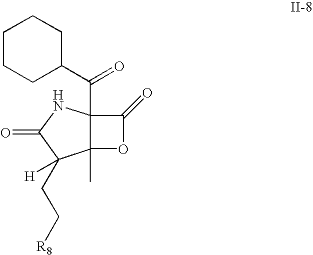

In some embodiments, R₈ can be, for example, hydrogen (II-8A), fluorine (II-8B), chlorine (II-8C), bromine (II-8D) and iodine (II-8E).

The following is exemplary stereochemistry for a compound having the structure of Formula II-8:

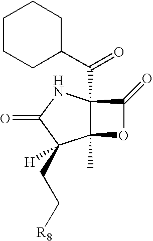

In some embodiments E₅ can be an amine oxide, giving rise to an oxime. An exemplary compound of Formula I has the following structure of Formula II-9:

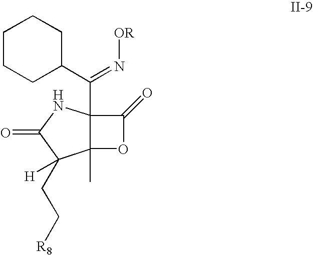

R₈ can be selected from the group consisting of hydrogen, fluorine, chlorine, bromine and iodine; R can be a hydrogen, or an optionally substituted substituent selected from the group consisting of alkyl, aryl, heteroaryl, and the like.

The following is exemplary stereochemistry for a compound having the structure of Formula II-9:

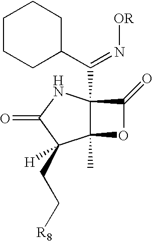

A further exemplary compound of Formula I has the following structure of Formula II-10:

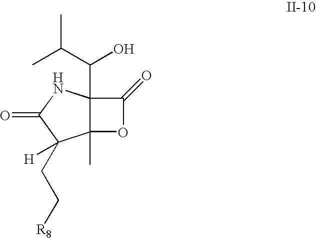

In some embodiments, for example, R₈ can be selected from the group consisting of hydrogen, fluorine, chlorine, bromine and iodine.

The following is exemplary stereochemistry for a compound having the structure of Formula II-10:

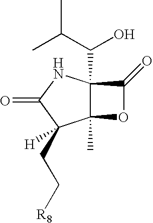

In some embodiments, E₅ can be NH₂. An exemplary compound of Formula I has the following structure of Formula II-11:

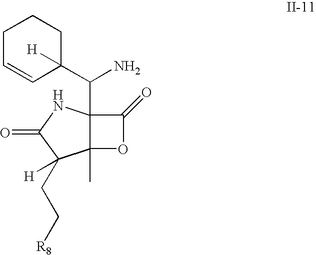

In some embodiments, for example, R₈ can be selected from the group consisting of hydrogen, fluorine, chlorine, bromine and iodine.

The following is exemplary stereochemistry for a compound having the structure of Formula II-11:

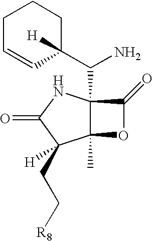

In some embodiments, at least one R₄ can be an optionally substituted cycloalkyl and E₅ can be NH₂. For example, R₄ can be cyclopropyl, cyclopentyl, cyclohexyl, cycloheptyl, and the like. An exemplary compound of Formula I has the following structure of Formula II-12:

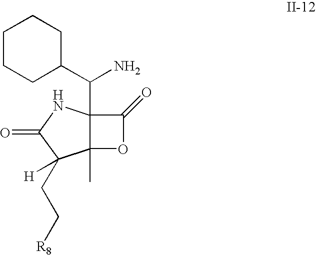

In some embodiments, for example, R₈ can be selected from the group consisting of hydrogen, fluorine, chlorine, bromine and iodine.

The following is exemplary stereochemistry for a compound having the structure of Formula II-12:

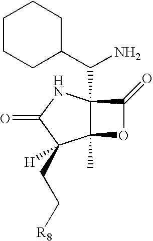

A further exemplary compound of Formula I has the following structure of Formula II-13:

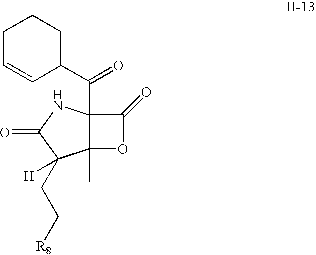

R₈ may include, for example, hydrogen (II-13A), fluorine (II-13B), chlorine (II-13C), bromine (II-13D) and iodine (II-13E).

The following is exemplary stereochemistry for a compound having the structure of Formula II-13:

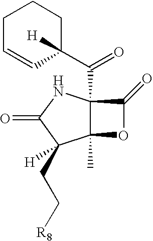

In another embodiment a compound of Formula I can have the following structure of Formula II-14:

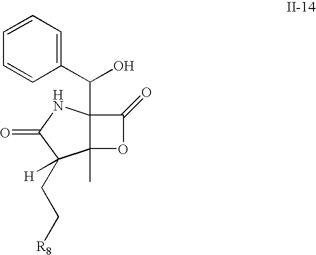

For example, R₈ can be selected from the group consisting of hydrogen, fluorine, chlorine, bromine and iodine.

The following is exemplary stereochemistry for a compound having the structure of Formula II-14:

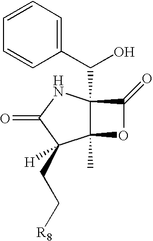

In another embodiment, for example, the radical R₄ of a compound of Formula II can be an optionally substituted cycloalkene. Furthermore, in some embodiments, the compounds of Formula II may include a hydroxy at E₅, for example. A further exemplary compound of Formula II has the following structure of Formula II-15:

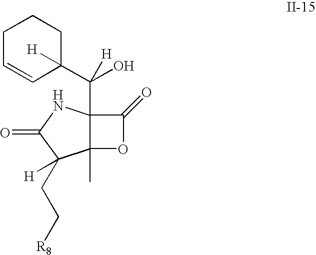

In some embodiments, for example, R₈ can be selected from the group consisting of hydrogen, fluorine, chlorine, bromine and iodine R₈ may include, for example, hydrogen, fluorine, chlorine, bromine and iodine.

Exemplary stereochemistry can be as follows:

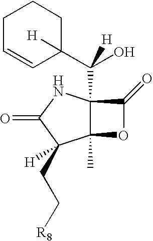

The following is exemplary stereochemistry for compounds having the structure of Formulae II-16, II-17, II-18, and II-19, respectively:

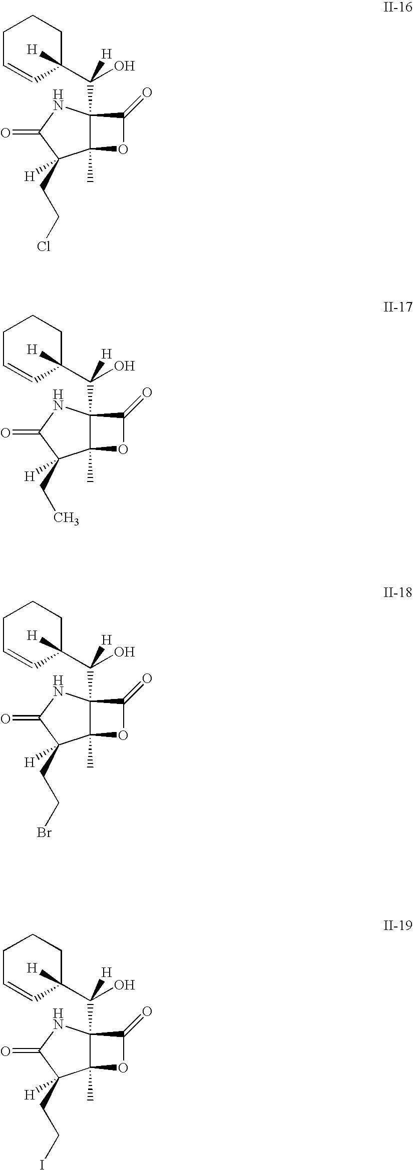

The compounds of Formulae II-16, II-17, II-18 and II-19 can be obtained by fermentation, synthesis, or semi-synthesis and isolated/purified as set forth below. Furthermore, the compounds of Formulae II-16, II-17, II-18 and II-19 can be used, and are referred to, as “starting materials” to make other compounds described herein.

In some embodiments, the compounds of Formula I, may include a methyl group as R₁, for example. A further exemplary compound, structure II-20, has the following structure and stereochemistry:

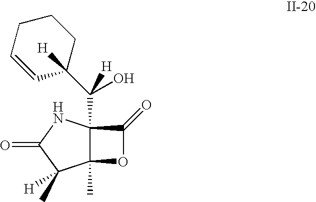

In some embodiments, the compounds of Formula I, may include hydroxyethyl as R₁, for example. A further exemplary compound, Formula II-21, has the following structure and stereochemistry:

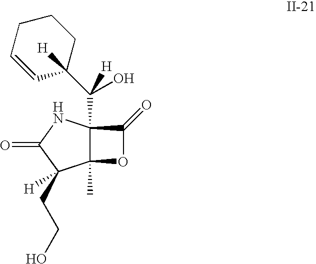

In some embodiments, the hydroxyl group of Formula II-21 can be esterified such that R₁ may include ethylpropionate, for example. An exemplary compound, structure II-22, has the following structure and stereochemistry:

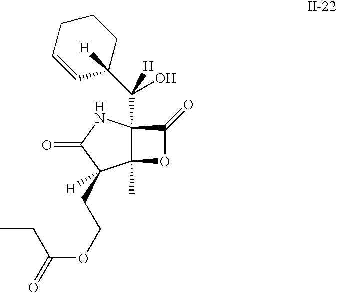

In some embodiments, the compounds of Formula I may include an ethyl group as R₃, for example. A further exemplary compound of Formula I has the following structure of Formula II-23:

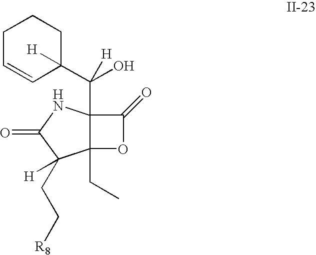

For example, R₈ can be selected from the group consisting of hydrogen, fluorine, chlorine, bromine and iodine. Exemplary stereochemistry can be as follows:

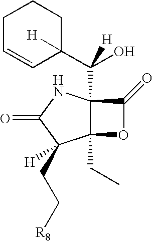

In some embodiments, the compounds of Formula II-23 may have the following structure and stereochemistry, exemplified by structure of Formula II-24C, where R₈ is chlorine:

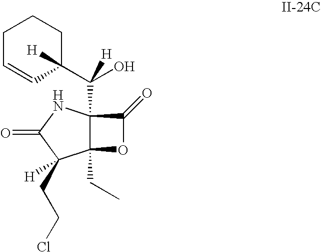

In some embodiments, the compounds of Formula II-15 may have the following stereochemistry, exemplified by the compound of Formula II-25, where R₈ is chlorine:

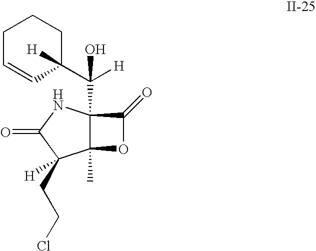

In some embodiments, the compound of Formula II-15 may have the following stereochemistry, exemplified by the compound of Formula II-26, where R₈ is chlorine:

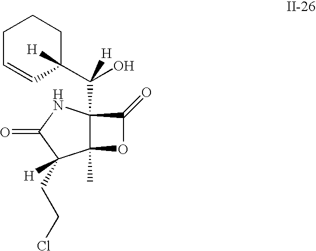

In some embodiments, the compound of Formula I may have the following structure and stereochemistry, exemplified by the structure of Formula II-27, where R₁ is ethyl:

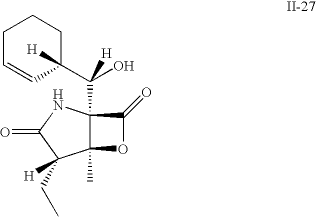

In some embodiments, the compound of Formula I may have the following structure and stereochemistry, exemplified by the structure of Formula II-28, where R₁ is methyl:

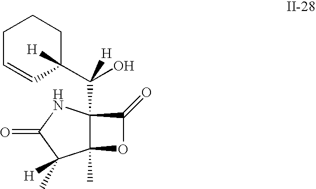

In some embodiments, the compounds of Formula I may include azidoethyl as R₁, for example. A further exemplary compound, Formula II-29, has the following structure and stereochemistry:

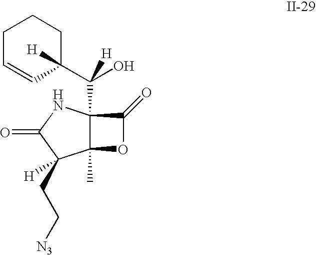

In some embodiments, the compounds of Formula I may include propyl as R₁, for example. A further exemplary compound, Formula II-30, has the following structure and stereochemistry:

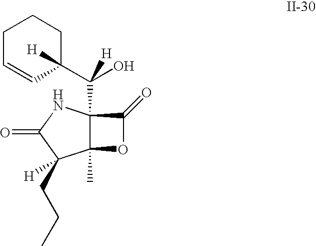

Still further exemplary compounds, Formulae II-31 and II-32, have the following structure and stereochemistry:

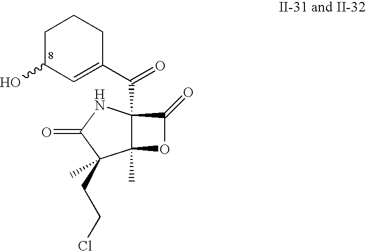

Other exemplary compounds, Formulae II-33, II-34, II-35 and II-36, have the following structure and stereochemistry:

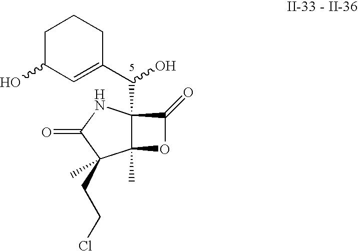

In some embodiments, the compound of Formula I may include cyanoethyl as R₁; for example, the compound of Formula II-37 has the following structure and stereochemistry:

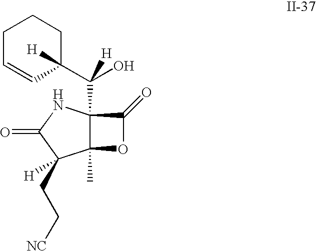

In another embodiment, the compound of Formula I may include ethylthiocyanate as R₁; for example, the compound of Formula II-38 has the following structure and stereochemistry:

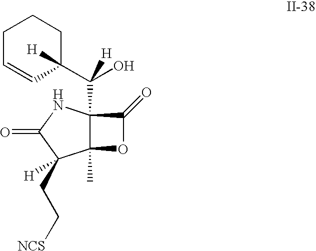

In some embodiments, the compounds of Formula I may include a thiol as R₁, for example. A further exemplary compound, Formula II-39, has the following structure and stereochemistry, where R═H, alkyl, aryl, or substituted alkyl or aryl:

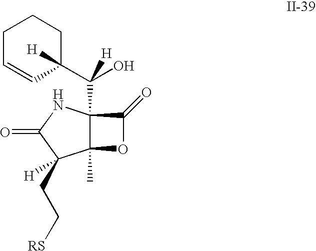

In a further exemplary compound, the sulfur of the compound of Formula II-39 can be oxidized to a sulfoxide (n=1) or sulfone (n=2), for example, as in the compound of structure II-40:

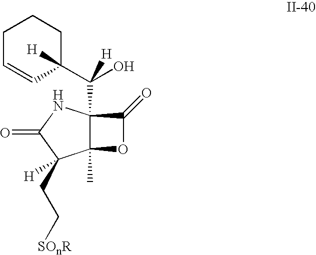

In some embodiments, the substituent R₁ of the compound of Formula I may include a leaving group, for example, a halogen, as in compounds of Formulae II-18 or II-19, or another leaving group, such as a sulfonate ester. One example is the methane sulfonate (mesylate) of Formula II-41:

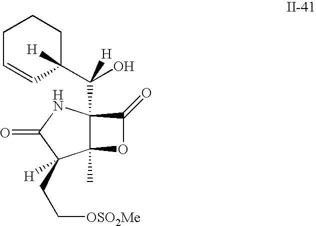

In some embodiments, the substituent R₁ of the compound of Formula I may include electron acceptors. The electron acceptor can be, for example, a Lewis acid, such as a boronic acid or ester. An exemplary compound, Formula II-42, has the following structure and stereochemistry, where n=0, 1, 2, 3, 4, 5, or 6, for example, and where R═H or alkyl, for example:

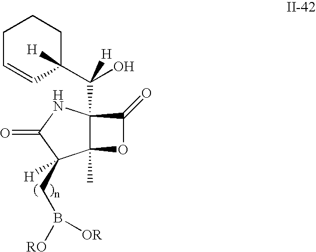

Further exemplary compounds of Formula II-42 are the compounds of Formula II-42A, where n=2 and R═H, and the compound of Formula II-42B, where n=1 and R═H:

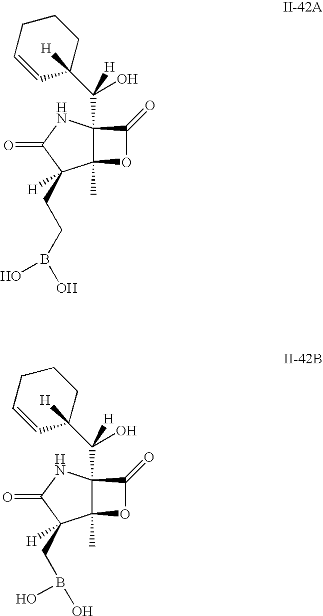

In some embodiments where the substituent R₁ of the compound of Formula I includes an electron acceptor, the electron acceptor can be, for example, a Michael acceptor. An exemplary compound, structure II-43 has the following structure, where n=0, 1, 2, 3, 4, 5, 6, and where Z is an electron withdrawing group, for example, CHO, COR, COOR, CONH₂, CN, NO₂, SOR, SO₂R, etc:

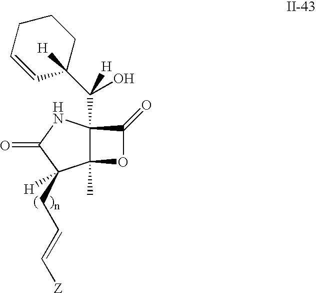

A further exemplary compound of Formula II-43 is the compound of structure II-43A, where n=1 and Z=CO₂CH₃:

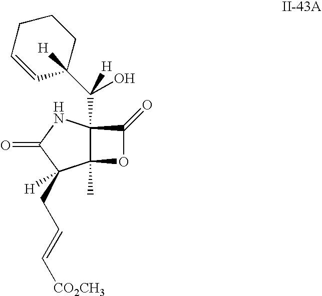

In some embodiments, the compounds can be prodrug esters or thioesters of the compounds of Formula I. For example, the compound of Formula II-44 (a prodrug thioester of the compound of structure II-16) has the following structure and stereochemistry:

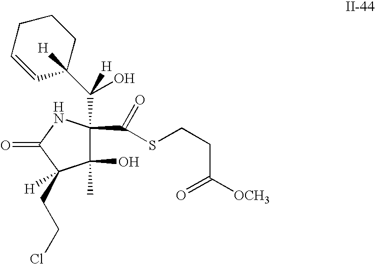

In some embodiments, the compounds of Formula I may include an alkenyl group as R₁, for example, ethylenyl. A further exemplary compound, Formula II-46, has the following structure and stereochemistry:

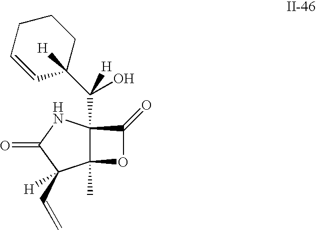

In some embodiments, the compounds can be prodrug esters or thioesters of the compounds of Formula I. For example, the compound of Formula II-47 (a prodrug thioester of the compound of structure II-17) has the following structure and stereochemistry:

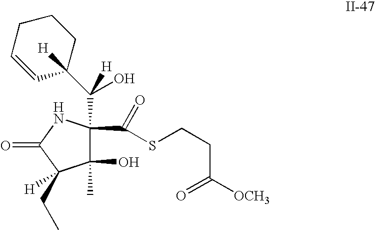

In some embodiments, the compounds can be prodrug esters or thioesters of the compounds of Formula I. For example, the compound of Formula II-48 has the following structure and stereochemistry:

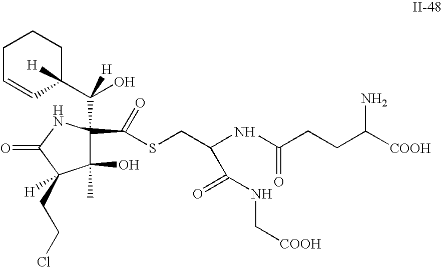

Another exemplary compound, structure II-49 has the following structure and stereochemistry:

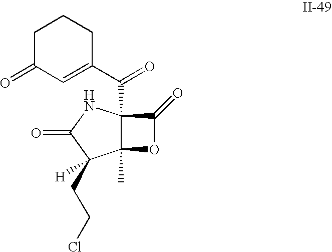

In some embodiments, the compound can be prodrug ester or thioester of the compounds of Formula I. For example, the compound of Formula II-50 (prodrug ester of the compound of Formula II-16) has the following structure and stereochemistry:

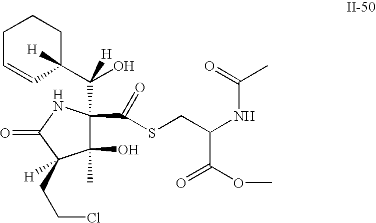

An exemplary compound of Formula I is the following Formula III-1, with and without exemplary stereochemistry:

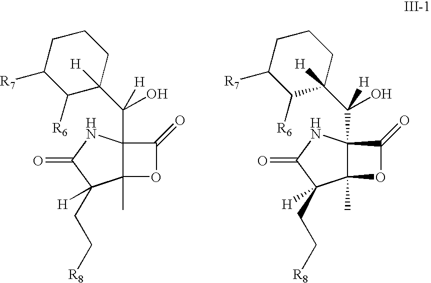

In some embodiments, for example, R₈ can be selected from the group consisting of hydrogen, fluorine, chlorine, bromine and iodine. The substituent(s) R₆ and R₇ may each separately be selected from a hydrogen, a halogen, a nitro, a cyano, or an optionally substituted substituent selected from the group consisting of C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxy carbonyl, alkoxy carbonylacyl, amino, aminocarbonyl, aminocarbonyloxy, azido, phenyl, hydroxy, alkylthio, arylthio, oxysulfonyl, carboxy, and halogenated alkyl including polyhalogenated alkyl. Further, R₆ and R₇ both can be the same or different.

For example, an exemplary compound of Formula I has the following Formula III-2:

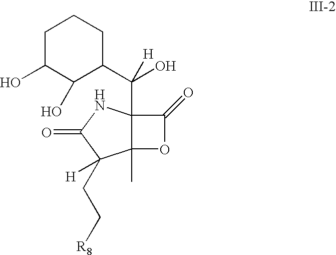

R₈ may include, for example, hydrogen, fluorine, chlorine, bromine and iodine.

Exemplary stereochemistry can be as follows:

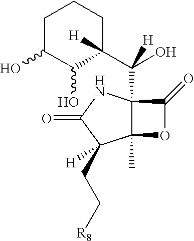

For example, an exemplary compound of Formula I has the following Formula III-3:

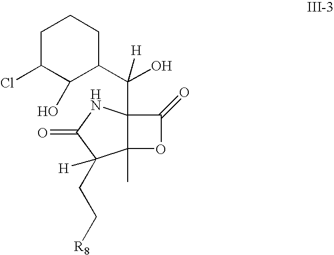

R₈ may include, for example, hydrogen (III-3A), fluorine (III-3B), chlorine (III-3C), bromine (III-3D) and iodine (III-3E).

Exemplary structure and stereochemistry can be as follows:

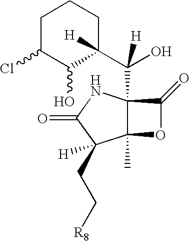

Additional exemplary structure and stereochemistry can be as follows:

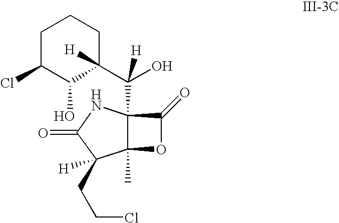

For example, an exemplary compound of Formula I has the following Formula III-4:

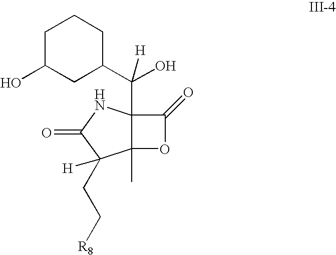

R₈ may include, for example, hydrogen, fluorine, chlorine, bromine and iodine.

Exemplary stereochemistry can be as follows:

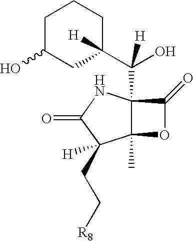

Certain embodiments also provide pharmaceutically acceptable salts and pro-drug esters or thioesters of the compound of Formulae I and II, and provide methods of obtaining and purifying such compounds by the methods disclosed herein.

The term “pro-drug,” especially when referring to a pro-drug ester of the compound of Formula I synthesized by the methods disclosed herein, refers to a chemical derivative of the compound that is rapidly transformed in vivo to yield the compound, for example, by hydrolysis in blood or inside tissues. The term “pro-drug ester” refers to derivatives of the compounds disclosed herein formed by the addition of any of several ester- or thioester-forming groups that are hydrolyzed under physiological conditions. Examples of pro-drug ester groups include pivoyloxymethyl, acetoxymethyl, phthalidyl, indanyl and methoxymethyl, as well as other such groups known in the art, including a (5-R-2-oxo-1,3-dioxolen-4-yl)methyl group. Other prodrugs can be prepared by preparing a corresponding thioester of the compound, for example, by reacting with an appropriate thiol, such as thiophenol, Cysteine or derivatives thereof, or propanethiol, for example. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design: Theory and Application”, edited by E. B. Roche, Pergamon Press: New York, 14-21 (1987) (providing examples of esters useful as prodrugs for compounds containing carboxyl groups). Each of the above-mentioned references is hereby incorporated by reference in its entirety.

The term “pharmaceutically acceptable salt,” as used herein, and particularly when referring to a pharmaceutically acceptable salt of a compound, including a compound of Formulae I and II, and Formula I and II as produced and synthesized by the methods disclosed herein, refers to any pharmaceutically acceptable salts of a compound, and preferably refers to an acid addition salt of a compound. Preferred examples of pharmaceutically acceptable salt are the alkali metal salts (sodium or potassium), the alkaline earth metal salts (calcium or magnesium), or ammonium salts derived from ammonia or from pharmaceutically acceptable organic amines, for example C₁-C₇ alkylamine, cyclohexylamine, triethanolamine, ethylenediamine or tris-(hydroxymethyl)-aminomethane. With respect to compounds synthesized by the method of this embodiment that are basic amines, the preferred examples of pharmaceutically acceptable salts are acid addition salts of pharmaceutically acceptable inorganic or organic acids, for example, hydrohalic, sulfuric, phosphoric acid or aliphatic or aromatic carboxylic or sulfonic acid, for example acetic, succinic, lactic, malic, tartaric, citric, ascorbic, nicotinic, methanesulfonic, p-toluensulfonic or naphthalenesulfonic acid.

Preferred pharmaceutical compositions disclosed herein include pharmaceutically acceptable salts and pro-drugs of a compound of compound of Formulae I and II obtained and purified by the methods disclosed herein. Accordingly, if the manufacture of pharmaceutical formulations involves intimate mixing of the pharmaceutical excipients and the active ingredient in its salt form, then it is preferred to use pharmaceutical excipients which are non-basic, that is, either acidic or neutral excipients.

It will be also appreciated that the phrase “compounds and compositions comprising the compound,” or any like phrase, is meant to encompass compounds in any suitable form for pharmaceutical delivery, as discussed in further detail herein. For example, in certain embodiments, the compounds or compositions comprising the same may include a pharmaceutically acceptable salt of the compound.

In one embodiment the compounds can be used to treat microbial diseases. Disease is meant to be construed broadly to cover infectious diseases, and also autoimmune diseases, non-infectious diseases and chronic conditions. In a preferred embodiment, the disease is caused by a microbe, such as a bacterium, a fungi, and protozoa, for example. The methods of use may also include the steps of administering a compound or composition comprising the compound to an individual with an infectious disease. The compound or composition can be administered in an amount effective to treat the particular infectious disease.

The infectious disease can be, for example, one caused by Bacillus, such as Tubercle Bacillus. The compound or composition can be administered with a pharmaceutically acceptable carrier, diluent, excipient, and the like.

The term “halogen atom,” as used herein, means any one of the radio-stable atoms of column 7 of the Periodic Table of the Elements, i.e., fluorine, chlorine, bromine, or iodine.

The term “alkyl,” as used herein, means any unbranched or branched, substituted or unsubstituted, fully saturated (no double or triple bonds) hydrocarbon group. The alkyl group may have 1 to 24 carbon atoms (whenever it appears herein, a numerical range such as “1 to 24” refers to each integer in the given range; e.g., “1 to 24 carbon atoms” means that the alkyl group may consist of 1 carbon atom, 2 carbon atoms, 3 carbon atoms, etc., up to and including 24 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds may be designated as “C₁₋₆ alkyl” or similar designations. By way of example only, “C₁₋₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, t-butyl, pentyl and hexyl. Typical alkyl groups include, but are in no way limited to, methyl, ethyl, propyl, isopropyl, butyl, isobutyl, t-butyl, pentyl, hexyl, and the like.

The term “substituted” has its ordinary meaning, as found in numerous contemporary patents from the related art. See, for example, U.S. Pat. Nos. 6,509,331; 6,506,787; 6,500,825; 5,922,683; 5,886,210; 5,874,443; and 6,350,759; all of which are incorporated herein in their entireties by reference. Specifically, the definition of substituted is as broad as that provided in U.S. Pat. No. 6,509,331, which defines the term “substituted alkyl” such that it refers to an alkyl group, preferably of from 1 to 10 carbon atoms, having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyacylamino, cyano, halogen, hydroxyl, carboxyl, carboxylalkyl, keto, thioketo, thiol, thioalkoxy, substituted thioalkoxy, thiocyanate, aryl, aryloxy, heteroaryl, heteroaryloxy, heterocyclic, heterocyclooxy, hydroxyamino, alkoxyamino, nitro, azido, boronic acid, boronic ester, —SO-alkyl, —SO-substituted alkyl, —SO-aryl, —SO-heteroaryl, —SO₂-alkyl, —SO₂-substituted alkyl, —SO₂-aryl, —SO₂-heteroaryl, —OSO-alkyl, —OSO-substituted alkyl, —OSO-aryl, —OSO-heteroaryl, —OSO₂-alkyl, —OSO₂-substituted alkyl, —OSO₂-aryl, and —OSO₂-heteroaryl. The other above-listed patents also provide standard definitions for the term “substituted” that are well-understood by those of skill in the art.

The term “cycloalkyl” as used herein, refers to any non-aromatic hydrocarbon ring, preferably having three to twelve atoms comprising the ring.

The term “acyl” as used herein, refers to alkyl or aryl groups derived from an oxoacid, with an acetyl group being preferred.

The term “alkoxycarbonylacyl” as used herein, refers to an acyl group substituted with an alkoxycarbonyl group. Typical alkoxycarbonylacyl groups include, but are in no way limited to, CH₃OC(O)CH₂C(O)—, CH₃CH₂CH₂OC(O)CH₂C(O)—, 4-ethoxycarbonylbenzoyl-, 4-methoxycarbonylbenzoyl-, 4-propoxycarbonylbenzoyl-, 3-tert-butoxycarbonylbenzoyl-, and the like.

The term “amino” as used herein, refers to amine radicals, wherein one or both hydrogen atoms are optionally replaced by substituents such as alkyl, and aryl groups. Typical amino groups include, but are in no way limited to, —NH₂, —NHMe, —NHEt, —NHCH₂phenyl, —N(Me)(phenyl), —N(Et)(Me), —N(Phenyl)(Et), —N(Et)(CH₂phenyl), —N(CH₂phenyl)(phenyl), and the like.

The term “aminocarbonyl” and as used herein, refers to a carbonyl substituted with an amino. Typical aminocarbonyl groups include, but are in no way limited to, —C(O)NH₂, —C(O)NHMe, —C(O)NHEt, —C(O)NHCH₂phenyl, —C(O)N(Me)(phenyl), —C(O)N(Et)(Me), —C(O)N(Phenyl)(Et), —C(O)N(Et)(CH₂phenyl), —C(O)N(CH₂phenyl)(phenyl), and the like.

The term “acyloxy” as used herein, refers to an acyl group attached to an oxygen with the oxygen being the attachment point. Typical acyloxy groups include, but are in no way limited to, MeC(O)O—, PhenylC(O)O—, and the like.

The term “alkenyl” as used herein, means any unbranched or branched, substituted or unsubstituted, unsaturated hydrocarbon including polyunsaturated hydrocarbons, with C₁-C₆ unbranched, mono-unsaturated and di-unsaturated, unsubstituted hydrocarbons being preferred, and mono-unsaturated, di-halogen substituted hydrocarbons being most preferred.

The term “cycloalkenyl” as used herein, refers to any non-aromatic hydrocarbon ring, preferably having five to twelve atoms comprising the ring and having at least one unsaturated bond.

The term “heterocycle” or “heterocyclic” refer to any non-aromatic cyclic compound containing one or more heteroatoms. In polycyclic ring systems, the one or more heteroatoms, may be present in only one of the rings. A heterocycle or heterocyclic group may be substituted or unsubstituted. The substituted heterocycle or heterocyclic group can be substituted with any substituent, including those described above and those known in the art.

The term “aryl” as used herein, refers to a carbocyclic (all carbon) ring or two or more fused rings (rings that share two adjacent carbon atoms) that have a fully delocalized pi-electron system. Typical aryl groups include, but are in no way limited to, benzene, naphthalene, azulene and the like. An aryl group may be substituted or unsubstituted. The substituted aryls can be substituted with any substituent, including those described above and those known in the art.

The term “heteroaryl” as used herein, refers to an aromatic heterocyclic group, whether one ring or multiple fused rings. In fused ring systems, the one or more heteroatoms, may be present in only one of the rings. The hetero atom is an element other than carbon, including but not limited to, nitrogen, oxygen and sulfur. Typical heteroaryl groups include, but are in no way limited to, indole, oxazole, benzoxazole, isoxazole, benzisoxazole, thiazole, benzothiazole, isothiazole, imidazole, benzimidazole, pyrazole, pyridazine, pyridine, pyrimidine, purine, pyrazine, pteridine, pyrrole, phenoxazole, oxazole, isoxazole, oxadiazole, benzopyrazole, indazole, quinolizine, cinnoline, phthalazine, quinazoline, quinoxaline, and the like. A heteroaryl group of this invention may be substituted or unsubstituted. The substituted heteroaryls can be substituted with any substituent, including those described above and those known in the art.

The term “alkoxy” as used herein, refers to any unbranched, or branched, substituted or unsubstituted, saturated or unsaturated ether, with C₁-C₆ unbranched, saturated, unsubstituted ethers being preferred, with methoxy being preferred, and also with dimethyl, diethyl, methyl-isobutyl, and methyl-tert-butyl ethers also being preferred.

The term “cycloalkoxy” as used herein, refers to any cycloalkyl attached to an oxygen atom with the oxygen being the attachment point to the rest of the molecule.

The term “arylalkoxy” as used herein, refers to an alkoxy group substituted with an aryl group. For example, arylalkoxy can be methoxy substituted with an aryl group, such as benzyloxy and the like.

The term “arylalkoxycarbonyl” as used herein, refers to an arylalkoxy group attached to a carbonyl group with the carbonyl being the attachment point to the rest of the molecule. Typical arylalkoxycarbonyl groups include, but are in no way limited to, benzyloxycarbonyl (i.e., PhenylCH₂OC(O)—) and the like.

The term “cycloalkyl” as used herein, refers to any non-aromatic hydrocarbon ring.

The term “alkoxycarbonyl” as used herein, refers to any linear, branched, cyclic, saturated, unsaturated, aliphatic or aryl alkoxy attached to a carbonyl group with the carbonyl group being the attachment point to the rest of the molecule. Typical alkoxycarbonyl groups include, but are in no way limited to, ethoxycarbonyl group, propyloxycarbonyl group, isopropyloxycarbonyl group, butoxycarbonyl group, sec-butoxycarbonyl group, tert-butoxycarbonyl group, cyclopentyloxycarbonyl group, cyclohexyloxycarbonyl group, benzyloxycarbonyl group, allyloxycarbonyl group, phenyloxycarbonyl group, pyridyloxycarbonyl group, and the like.

The term “alkoxycarbonyloxy” as used herein, refers to an alkoxycarbonyl group attached to an oxygen with the oxygen being the attachment point to the rest of the molecule. Typical alkoxycarbonyloxy groups include, but are in no way limited to, MeOC(O)O—, methoxycarbonyloxy group, ethoxycarbonyloxy group, propyloxycarbonyloxy group, isopropyloxycarbonyloxy group, butoxycarbonyloxy group, sec-butoxycarbonyloxy group, tert-butoxycarbonyloxy group, cyclopentyloxycarbonyloxy group, cyclohexyloxycarbonyloxy group, allyloxycarbonyloxy group, benzyloxycarbonyloxy group and the like. Additionally, alkoxycarbonyloxy groups refer to aryloxy and heteroaryloxy groups such as, phenyloxycarbonyloxy group, pyridyloxycarbonyloxy group, and the like.

The terms “pure,” “purified,” “substantially purified,” and “isolated” as used herein refer to the compound of the embodiment being free of other, dissimilar compounds with which the compound, if found in its natural state, would be associated in its natural state. In certain embodiments described as “pure,” “purified,” “substantially purified,” or “isolated” herein, the compound may comprise at least 0.5%, 1%, 5%, 10%, or 20%, and most preferably at least 50% or 75% of the mass, by weight, of a given sample.

The terms “derivative,” “variant,” or other similar term refers to a compound that is an analog of the other compound.

Certain of the compounds of any of Formulae I and II can be obtained and purified or can be obtained via semi-synthesis from purified compounds as set forth herein. Generally, without being limited thereto, the compounds of Formula II-15, preferably, Formulae II-16 (Salinosporamide A), II-17, II-18 and II-19, can be obtained synthetically or by fermentation. Exemplary fermentation procedures are provided below. Further, the compounds of structure II-15, preferably, Formulae II-16, II-17, II-18 and II-19 can be used as starting compounds in order to obtain/synthesize various of the other compounds described herein. Exemplary non-limiting syntheses are provided herein.

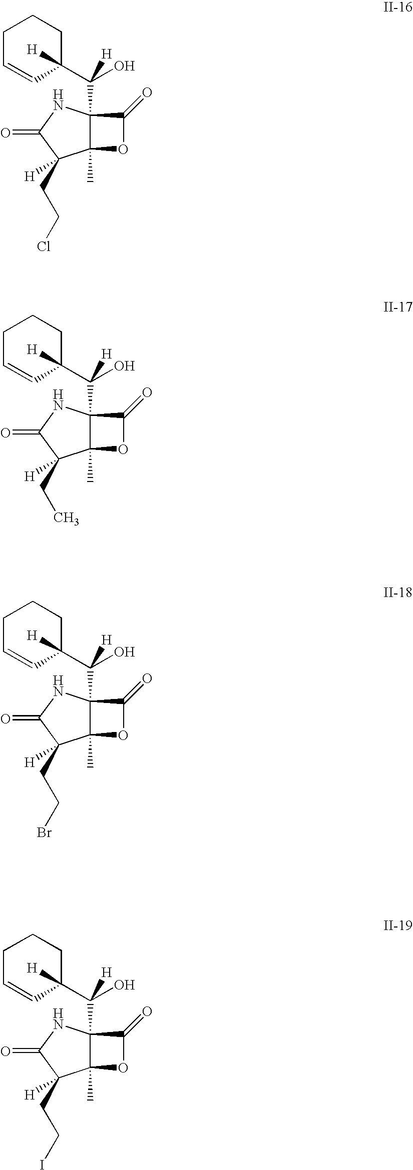

The compound of Formula II-16 may be produced through a high-yield saline fermentation (˜350-400 mg/L) and modifications of the conditions have yielded new analogs in the fermentation extracts. Additional analogs can be generated through directed biosynthesis. Directed biosynthesis is the modification of a natural product by adding biosynthetic precursor analogs to the fermentation of producing microorganisms (Lam, et al., J Antibiot (Tokyo) 44:934 (1991), Lam, et al., J Antibiot (Tokyo) 54:1 (2001); which is hereby incorporated by reference in its entirety).

Exposing the producing culture to analogs of acetic acid, phenylalanine, valine, butyric acid, shikimic acid, and halogens, preferably, other than chlorine, can lead to the formation of new analogs. The new analogs produced can be easily detected in crude extracts by HPLC and LC-MS. For example, after manipulating the medium with different concentrations of sodium bromide, a bromo-analog, the compound of Formula II-18, was successfully produced in shake-flask culture at a titer of 14 mg/L.

A second approach to generate analogs is through biotransformation. Biotransformation reactions are chemical reactions catalyzed by enzymes or whole cells containing these enzymes. Zaks, A., Curr Opin Chem Biol 5:130 (2001). Microbial natural products are ideal substrates for biotransformation reactions as they are synthesized by a series of enzymatic reactions inside microbial cells. Riva, S., Curr Opin Chem Biol 5:106 (2001).

Given the structure of the described compounds, including those of Formula I-15, for example, the possible biosynthetic origins are acetyl-CoA, ethylmalonyl-CoA, phenylalanine and chlorine. Ethylmalonyl-CoA is derived from butyryl-CoA, which can be derived either from valine or crotonyl-CoA. Liu, et al., Metab Eng 3:40 (2001). Phenylalanine is derived from shikimic acid.

Alternatively, compounds such as structure II-16 and its analogs may be produced synthetically, e.g., such as described in U.S. application Ser. No. 11/697,689, which is incorporated by reference in its entirety.

### Production of Compounds of Formulae I-7, II-16, II-17, II-18, II-20, II-24C, II-26, II-27 and II-28

The production of compounds of Formulae I-7, II-16, II-17, II-18, II-20, II-24C, II-26, II-27 and II-28 can be carried out by cultivating strain CNB476 and strain NPS21184, a natural variant of strain CNB476, in a suitable nutrient medium under conditions described herein, preferably under submerged aerobic conditions, until a substantial amount of compounds are detected in the fermentation; harvesting by extracting the active components from the fermentation broth with a suitable solvent; concentrating the solvent containing the desired components; then subjecting the concentrated material to chromatographic separation to isolate the compounds from other metabolites also present in the cultivation medium.

The culture (CNB476) was deposited on Jun. 20, 2003 with the American Type Culture Collection (ATCC) in Rockville, Md. and assigned the ATCC patent deposition number PTA-5275. Strain NPS21184, a natural variant of strain CNB476, was derived from strain CNB476 as a single colony isolate. Strain NPS21184 has been deposited to ATCC on Apr. 27, 2005. The ATCC deposit meets all of the requirements of the Budapest treaty. The culture is also maintained at and available from Nereus Pharmaceutical Culture Collection at 10480 Wateridge Circle, San Diego, Calif. 92121. In addition to the specific microorganism described herein, it should be understood that mutants, such as those produced by the use of chemical or physical mutagens including X-rays, etc. and organisms whose genetic makeup has been modified by molecular biology techniques, may also be cultivated to produce the starting compounds of Formulae II-16, II-17, and II-18.

### Fermentation of Strain CNB476 and Strain NPS21184

Production of compounds can be achieved at temperature conducive to satisfactory growth of the producing organism, e.g. from 16° C. to 40° C., but it is preferable to conduct the fermentation at 22° C. to 32° C. The aqueous medium can be incubated for a period of time necessary to complete the production of compounds as monitored by high pressure liquid chromatography (HPLC), preferably for a period of about 2 to 10 days, on a rotary shaker operating at about 50 rpm to 400 rpm, preferably at 150 rpm to 250 rpm, for example. The production of the compounds can also be achieved by cultivating the production strain in a bioreactor, such as a fermentor system that is suitable for the growth of the production strain.

Growth of the microorganisms can be achieved by one of ordinary skill of the art by the use of appropriate medium. Broadly, the sources of carbon include glucose, fructose, mannose, maltose, galactose, mannitol and glycerol, other sugars and sugar alcohols, starches and other carbohydrates, or carbohydrate derivatives such as dextran, cerelose, as well as complex nutrients such as oat flour, corn meal, millet, corn, and the like. The exact quantity of the carbon source that is utilized in the medium will depend in part, upon the other ingredients in the medium, but an amount of carbohydrate between 0.5 to 25 percent by weight of the medium can be satisfactorily used, for example. These carbon sources can be used individually or several such carbon sources can be combined in the same medium, for example. Certain carbon sources are preferred as hereinafter set forth.

The sources of nitrogen include amino acids such as glycine, arginine, threonine, methionine and the like, ammonium salt, as well as complex sources such as yeast extracts, corn steep liquors, distiller solubles, soybean meal, cotttonseed meal, fish meal, peptone, and the like. The various sources of nitrogen can be used alone or in combination in amounts ranging from 0.5 to 25 percent by weight of the medium, for example.

Among the nutrient inorganic salts, which can be incorporated in the culture media, are the customary salts capable of yielding sodium, potassium, magnesium, calcium, phosphate, sulfate, chloride, carbonate, and like ions. Also included are trace metals such as cobalt, manganese, iron, molybdenum, zinc, cadmium, and the like.

### Biological Activity and Uses of Compounds

Some embodiments relate to methods of treating infectious diseases, particularly those affecting humans. The methods may include, for example, the step of administering an effective amount of a compound disclosed herein.

The compounds have proteasome inhibitory activity. The proteasome inhibitory activity may, in whole or in part, contribute to the ability of the compounds to act as anti-microbial agents.

The proteasome is a multisubunit protease that degrades intracellular proteins through its chymotrypsin-like, trypsin-like and peptidylglutamyl-peptide hydrolyzing (PGPH; and also know as the caspase-like activity) activities. The 26S proteasome contains a proteolytic core called the 20S proteasome and one or two 19S regulatory subunits. The 20S proteasome is responsible for the proteolytic activity against many substrates including damaged proteins, the transcription factor NF-κB and its inhibitor IκB, signaling molecules, tumor suppressors and cell cycle regulators. There are three distinct protease activities within the proteasome: 1) chymotrypsin-like; 2) trypsin-like; and the 3) peptidyl glutamyl peptide hydrolyzing (PGPH) activity.

As an example, compounds of Formula II-16 were more potent (EC₅₀ 2 nM) at inhibiting the chymotrypsin-like activity of rabbit muscle proteasomes than Omuralide (EC₅₀ 52 nM) and also inhibited the chymotrypsin-like activity of human erythrocyte derived proteasomes (EC₅₀ ˜250 pM). Compounds of Formula II-16 exhibit a significant preference for inhibiting chymotrypsin-like activity of the proteasome over inhibiting the catalytic activity of chymotrypsin. Compounds of Formula II-16 also exhibit low nM trypsin-like inhibitory activity (˜10 nM), but are less potent at inhibiting the PGPH activity of the proteasome (EC₅₀ ˜350 nM).

Additional studies have characterized the effects of compounds described herein, including studies of Formula II-16 on the NF-κB/IκB signaling pathway. Treatment of HEK293 cells (human embryonic kidney) with Tumor Necrosis Factor-alpha (TNF-α) induces phosphorylation and proteasome-mediated degradation of IκBα followed by NF-κB activation. To confirm proteasome inhibition, HEK293 cells were pre-treated for 1 hour with compounds of Formula II-16 followed by TNF-α stimulation. Treatment with compounds of Formula II-16 promoted the accumulation of phosphorylated IκBα suggesting that the proteasome-mediated IκBα degradation was inhibited.

Furthermore, a stable HEK293 clone (NF-κB/Luc 293) was generated carrying a luciferase reporter gene under the regulation of 5×NF-κB binding sites. Stimulation of NF-κB/Luc 293 cells with TNF-α increases luciferase activity as a result of NF-κB activation while pretreatment with compounds of Formula II-16 decreases activity. Western blot analyses demonstrated that compounds of Formula II-16 promoted the accumulation of phosphorylated-IκBα and decreased the degradation of total IκBα in the NF-κB/Luc 293 cells. Compounds of Formula II-16 were also shown to increase the levels of the cell cycle regulatory proteins, p21 and p27.

### Anti-Tuberculosis Activity

Another potential application for proteasome inhibitors comes from recent studies on sensitivity of Mycobacterium tuberculosis to nitric oxide and other reactive nitrogen intermediates. Pieters, et al., Science 301: 1900 (2003) and Darwin, et al., Science 301: 1963 (2003). Tuberculosis infection is caused when small droplets containing Mycobacterium tuberculosis are inhaled and lodge in the lungs where they are internalized by alveolar macrophages. Within the macrophage phagosomes Mycobacterium tuberculosis thrives by actively blocking their fusion with lysosomes thus avoiding destruction.

The host organisms ability to generate reactive nitrogen intermediates plays a key role in controlling Mycobacterium tuberculosis growth. It was reasoned that because Mycobacterium tuberculosis can survive decades in host organisms the bacteria must have a mechanism to ameliorate exposure to reactive nitrogen intermediates. It has been shown that mycobacterial proteasomes can counter the destructive effects of reactive nitrogen intermediates thereby allowing the bacteria to survive. It is suggested that inhibition of the proteasome of Mycobacterium tuberculosis can prevent resistance of the bacteria to reactive nitrogen intermediates.

Salinosporamide A can increase sensitivity of Mycobacterium tuberculosis to nitric oxide and reactive nitrogen intermediates by inhibiting the proteasome of mycobacterium tuberculosis. The activity of Salinosporamide A in inhibition of the proteasome of mycobacterium tuberculosis is tested by measuring proteasomal protease activity in cell lysates.

Salinosporamide A can be assayed in liquid culture to test its ability to inhibit recovery of wild-type Mycobacterium tuberculosis from nitrite-mediated injury. Salinosporamide A can block the ability of Mycobacterium tuberculosis to recover from nitrate-mediated injury.

In survival assays Salinosporamide A can be evaluated based on the growth of Mycobacterium tuberculosis on agar plates. Salinosporamide A can augment the antimycobacterial effect of nitrite by irreversibly inhibiting proteasomal protease, this in turn can increases the when the inhibitors and nitrite are removed simultaneously by plating bacteria on agar after 6 days of exposure. Salinosporamide A can augment the antimycobacterial effect of nitrite, if present, after nitrite mediated injury. Salinosporamide A can also enhance the antimycobacterial effect when added along with nitrite at day 0 or after the subculture on day 6, plating on day 10. Salinosporamide A can increase the antimycobacterial activity of nitrite when Mycobacterium tuberculosis is given time to recover during a 4-day period of subculture at pH 6.5 before being plated. Salinosporamide A can also be effective if added at the time of subculture.

### Pharmaceutical Compositions

In one embodiment, the compounds disclosed herein are used in pharmaceutical compositions. The compounds preferably can be produced by the methods disclosed herein. The compounds can be used, for example, in pharmaceutical compositions comprising a pharmaceutically acceptable carrier prepared for storage and subsequent administration. Also, embodiments relate to a pharmaceutically effective amount of the products and compounds disclosed above in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), which is incorporated herein by reference in its entirety. Preservatives, stabilizers, dyes and even flavoring agents can be provided in the pharmaceutical composition. For example, sodium benzoate, ascorbic acid and esters of p-hydroxybenzoic acid can be added as preservatives. In addition, antioxidants and suspending agents can be used.

The compositions can be formulated and used as tablets, capsules, or elixirs for oral administration; suppositories for rectal administration; sterile solutions, suspensions for injectable administration; patches for transdermal administration, and sub-dermal deposits and the like. Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. If desired, absorption enhancing preparations (for example, liposomes), can be utilized.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate. Dragee cores are provided with suitable coatings. For this purpose, concentrated sugar solutions can be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses. For this purpose, concentrated sugar solutions can be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses. Such formulations can be made using methods known in the art (see, for example, U.S. Pat. Nos. 5,733,888 (injectable compositions); 5,726,181 (poorly water soluble compounds); 5,707,641 (therapeutically active proteins or peptides); 5,667,809 (lipophilic agents); 5,576,012 (solubilizing polymeric agents); 5,707,615 (anti-viral formulations); 5,683,676 (particulate medicaments); 5,654,286 (topical formulations); 5,688,529 (oral suspensions); 5,445,829 (extended release formulations); 5,653,987 (liquid formulations); 5,641,515 (controlled release formulations) and 5,601,845 (spheroid formulations); all of which are incorporated herein by reference in their entireties.

Further disclosed herein are various pharmaceutical compositions well known in the pharmaceutical art for uses that include topical, intraocular, intranasal, and intraauricular delivery. Pharmaceutical formulations include aqueous ophthalmic solutions of the active compounds in water-soluble form, such as eyedrops, or in gellan gum (Shedden et al., Clin. Ther., 23(3):440-50 (2001)) or hydrogels (Mayer et al., Opthalmologica, 210(2):101-3 (1996)); ophthalmic ointments; ophthalmic suspensions, such as microparticulates, drug-containing small polymeric particles that are suspended in a liquid carrier medium (Joshi, A. 1994 J Ocul Pharmacol 10:29-45), lipid-soluble formulations (Alm et al., Prog. Clin. Biol. Res., 312:447-58 (1989)), and microspheres (Mordenti, Toxicol. Sci., 52(1):101-6 (1999)); and ocular inserts. All of the above-mentioned references, are incorporated herein by reference in their entireties. Such suitable pharmaceutical formulations are most often and preferably formulated to be sterile, isotonic and buffered for stability and comfort. Pharmaceutical compositions may also include drops and sprays often prepared to simulate in many respects nasal secretions to ensure maintenance of normal ciliary action. As disclosed in Remington's Pharmaceutical Sciences (Mack Publishing, 18^{th} Edition), which is incorporated herein by reference in its entirety, and well-known to those skilled in the art, suitable formulations are most often and preferably isotonic, slightly buffered to maintain a pH of 5.5 to 6.5, and most often and preferably include anti-microbial preservatives and appropriate drug stabilizers. Pharmaceutical formulations for intraauricular delivery include suspensions and ointments for topical application in the ear. Common solvents for such aural formulations include glycerin and water.

To formulate the compounds of Formulae I and II as an anti-cancer agent, known surface active agents, excipients, smoothing agents, suspension agents and pharmaceutically acceptable film-forming substances and coating assistants, and the like can be used. Preferably alcohols, esters, sulfated aliphatic alcohols, and the like can be used as surface active agents; sucrose, glucose, lactose, starch, crystallized cellulose, mannitol, light anhydrous silicate, magnesium aluminate, magnesium methasilicate aluminate, synthetic aluminum silicate, calcium carbonate, sodium acid carbonate, calcium hydrogen phosphate, calcium carboxymethyl cellulose, and the like can be used as excipients; magnesium stearate, talc, hardened oil and the like can be used as smoothing agents; coconut oil, olive oil, sesame oil, peanut oil, soya can be used as suspension agents or lubricants; cellulose acetate phthalate as a derivative of a carbohydrate such as cellulose or sugar, or methylacetate-methacrylate copolymer as a derivative of polyvinyl can be used as suspension agents; and plasticizers such as ester phthalates and the like can be used as suspension agents. In addition to the foregoing preferred ingredients, sweeteners, fragrances, colorants, preservatives and the like can be added to the administered formulation of the compound produced by the method of the embodiment, particularly when the compound is to be administered orally.

When used as an anti-cancer compound, for example, the compounds of Formulae I and II or compositions including compounds of Formulae I and II can be administered by either oral or non-oral pathways. When administered orally, it can be administered in capsule, tablet, granule, spray, syrup, or other such form. When administered non-orally, it can be administered as an aqueous suspension, an oily preparation or the like or as a drip, suppository, salve, ointment or the like, when administered via injection, subcutaneously, intraperitoneally, intravenously, intramuscularly, or the like.

In one embodiment, the anti-cancer agent can be mixed with additional substances to enhance their effectiveness.

### Methods of Administration

In an alternative embodiment, the disclosed chemical compounds and the disclosed pharmaceutical compositions are administered by a particular method as an anti-cancer, anti-microbial or anti-inflammatory. Such methods include, among others, (a) administration though oral pathways, which administration includes administration in capsule, tablet, granule, spray, syrup, or other such forms; (b) administration through non-oral pathways, which administration includes administration as an aqueous suspension, an oily preparation or the like or as a drip, suppository, salve, ointment or the like; administration via injection, subcutaneously, intraperitoneally, intravenously, intramuscularly, intradermally, or the like; as well as (c) administration topically, (d) administration rectally, or (e) administration vaginally, as deemed appropriate by those of skill in the art for bringing the compound of the present embodiment into contact with living tissue; and (f) administration via controlled released formulations, depot formulations, and infusion pump delivery. As further examples of such modes of administration and as further disclosure of modes of administration, disclosed herein are various methods for administration of the disclosed chemical compounds and pharmaceutical compositions including modes of administration through intraocular, intranasal, and intraauricular pathways.

The pharmaceutically effective amount of the compositions that include the described compounds required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. In a typical embodiment, a compound represented by Formulae I and II can be administered to a patient in need of an anti-cancer agent, until the need is effectively reduced or preferably removed.

In practicing the methods of the embodiment, the products or compositions can be used alone or in combination with one another, or in combination with other therapeutic or diagnostic agents. These products can be utilized in vivo, ordinarily in a mammal, preferably in a human, or in vitro. In employing them in vivo, the products or compositions can be administered to the mammal in a variety of ways, including parenterally, intravenously, subcutaneously, intramuscularly, colonically, rectally, vaginally, nasally or intraperitoneally, employing a variety of dosage forms. Such methods may also be applied to testing chemical activity in vivo.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved or adverse side effects disappear. The dosage may range broadly, depending upon the desired affects and the therapeutic indication. Typically, dosages can be between about 10 mg/kg and 100 mg/kg body weight, preferably between about 100 mg/kg and 10 mg/kg body weight. Alternatively dosages can be based and calculated upon the surface area of the patient, as understood by those of skill in the art. Administration is preferably oral on a daily or twice daily basis.

The exact formulation, route of administration and dosage can be chosen by the individual physician in view of the patient's condition. See for example, Fingl et al., in The Pharmacological Basis of Therapeutics, 1975, which is incorporated herein by reference in its entirety. It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above can be used in veterinary medicine.

Depending on the specific conditions being treated, such agents can be formulated and administered systemically or locally. A variety of techniques for formulation and administration can be found in Remington's Pharmaceutical Sciences, 18th Ed., Mack Publishing Co., Easton, Pa. (1990), which is incorporated herein by reference in its entirety. Suitable administration routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections.

For injection, the agents of the embodiment can be formulated in aqueous solutions, preferably in physiologically compatible buffers such as Hanks' solution, Ringer's solution, or physiological saline buffer. For such transmucosal administration, penetrants appropriate to the barrier to be permeated are used in the formulation. Such penetrants are generally known in the art. Use of pharmaceutically acceptable carriers to formulate the compounds herein disclosed for the practice of the embodiment into dosages suitable for systemic administration is within the scope of the embodiment. With proper choice of carrier and suitable manufacturing practice, the compositions disclosed herein, in particular, those formulated as solutions, can be administered parenterally, such as by intravenous injection.

The compounds can be formulated readily using pharmaceutically acceptable carriers well known in the art into dosages suitable for oral administration. Such carriers enable the compounds of the embodiment to be formulated as tablets, pills, capsules, liquids, gels, syrups, slurries, suspensions and the like, for oral ingestion by a patient to be treated.

Agents intended to be administered intracellularly can be administered using techniques well known to those of ordinary skill in the art. For example, such agents can be encapsulated into liposomes, then administered as described above. All molecules present in an aqueous solution at the time of liposome formation are incorporated into the aqueous interior. The liposomal contents are both protected from the external micro-environment and, because liposomes fuse with cell membranes, are efficiently delivered into the cell cytoplasm. Additionally, due to their hydrophobicity, small organic molecules can be directly administered intracellularly.

Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration can be in the form of tablets, dragees, capsules, or solutions. The pharmaceutical compositions can be manufactured in a manner that is itself known, for example, by means of conventional mixing, dissolving, granulating, dragee-making, levitating, emulsifying, encapsulating, entrapping, or lyophilizing processes.

Compounds disclosed herein can be evaluated for efficacy and toxicity using known methods. For example, the toxicology of a particular compound, or of a subset of the compounds, sharing certain chemical moieties, can be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, can be determined using known methods. The efficacy of a particular compound can be established using several art recognized methods, such as in vitro methods, animal models, or human clinical trials. Art-recognized in vitro models exist for nearly every class of condition, including the conditions abated by the compounds disclosed herein, including cancer, cardiovascular disease, and various immune dysfunction, and infectious diseases. Similarly, acceptable animal models can be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

In the case of using a compound produced by methods of the embodiment as a biochemical test reagent, the compound produced by methods of the embodiment inhibits the progression of the disease when it is dissolved in an organic solvent or hydrous organic solvent and it is directly applied to any of various cultured cell systems. Usable organic solvents include, for example, methanol, methylsulfoxide, and the like. The formulation can, for example, be a powder, granular or other solid inhibitor, or a liquid inhibitor prepared using an organic solvent or a hydrous organic solvent. While a preferred concentration of the compound produced by the method of the embodiment for use as an anticancer compound is generally in the range of about 1 to about 100 μg/mL, the most appropriate use amount varies depending on the type of cultured cell system and the purpose of use, as will be appreciated by persons of ordinary skill in the art. Also, in certain applications it can be necessary or preferred to persons of ordinary skill in the art to use an amount outside the foregoing range.

As will be understood by one of skill in the art, “need” is not an absolute term and merely implies that the patient can benefit from the treatment of the anti-infective agent in use. By “patient” what is meant is an organism that can benefit by the use of an anti-infective agent. For example, any organism with an infectious disease, such as, Tuberculosis. In one embodiment, the patient's health may not require that an anti-infective agent be administered, however, the patient may still obtain some benefit by the reduction of the level of bacteria in the patient, and thus be in need. In one embodiment, the patient's health may not require that an anti-infective agent be administered, however, the spread of the infection from the patient to an individual who does not have the infection can be prevented by administration of the anti-infective agent to the patient. In another embodiment, the anti-infective agent can be administered to an individual in a profalactive preventative measure. In still further embodiments, the anti-infective agent is effective against a broad spectrum of infections. Examples of infections, against which the compounds can be effective include Bacteremia, Botulism, Brucellosis, Clostridium Difficile, Campylobacter Infection, Cat Scratch Disease, Chancroid, Chlamydia, Cholera, Clostridium Perfringens, Bacterial Conjunctivitis, Diphtheria, E. Coli Infections, Ehrlichiosis, Epididymitis, Gardnerella, Gas Gangrene, Gonorrhea, Helicobacter Pylori, Haemophilus, Influenzae B, Impetigo, Intertrigo, Leprosy, Listeriosis, Lyme Disease, Methicillin Resistant Staphylococcus Aureus, Orchitis, Osteomyelitis, Otitis, Media Pertussis, Plague, Pneumonia, Prostatitis Pyelonephritis, Q Fever, Rocky Mountain Spotted Fever, Salmonellosis, Scarlet Fever, Sepsis, Shigellosis, Staphylococcal Infections, Streptococcal Infections, Syphilis, Tetanus, Toxic Shock Syndrome, Trachoma, Traveller's Diarrhea, Tuberculosis, Tularemia, Typhoid Fever, Typhus Fever, Urinary Tract Infections, Bacterial Vaginosis, Pertussis, Yersiniosis, Sleeping Sickness, African trypanosomiasis, malaria, candidiasis, histoplasmosis, blastomycosis, coccidioidomycosis, aspergillisis, mucormycosis and the like.

“Therapeutically effective amount,” “pharmaceutically effective amount,” or similar term, means that amount of drug or pharmaceutical agent that will result in a biological or medical response of a cell, tissue, system, animal, or human that is being sought. In a preferred embodiment, the medical response is one sought by a researcher, veterinarian, medical doctor, or other clinician.

In one embodiment, a described compound, preferably a compound having any one of Formulas I and II, including those as described herein, is considered an effective anti-infective agent if the compound can influence 10% of the bacterial cells, for example. In a more preferred embodiment, the compound is effective if it can influence 10 to 50% of the bacterial cells. In an even more preferred embodiment, the compound is effective if it can influence 50-80% of the bacterial cells. In an even more preferred embodiment, the compound is effective if it can influence 80-95% of the bacterial cells. In an even more preferred embodiment, the compound is effective if it can influence 95-99% of the bacterial cells. “Influence” is defined by the mechanism of action for each compound. For example, if a compound prevents the proliferation of bacterial cells, then influence is a measure of prevention of bacterial cell proliferation. Not all mechanisms of action need be at the same percentage of effectiveness. In an alternative embodiment, a low percentage effectiveness can be desirable if the lower degree of effectiveness is offset by other factors, such as the specificity of the compound, for example. Thus a compound that is only 10% effective, for example, but displays little in the way of harmful side-effects to the host, or non-harmful microbes or cells, can still be considered effective.

The following non-limiting examples are meant to describe the preferred embodiments of the methods. Variations in the details of the particular methods employed and in the precise chemical compositions obtained will undoubtedly be appreciated by those of skill in the art.

### EXAMPLES

### Example 1

### Fermentation of Compound of Formulae I-7, II-16, II-17, II-20, II-24C, II-26 and II-28 Using Strain CNB476

Strain CNB476 was grown in a 500-mL flask containing 100 mL of vegetative medium consisting of the following per liter of deionized water: glucose, 4 g; Bacto tryptone, 3 g; Bacto casitone, 5 g; and synthetic sea salt (Instant Ocean, Aquarium Systems), 30 g. The first seed culture was incubated at 28° C. for 3 days on a rotary shaker operating at 250 rpm. Five mL each of the first seed culture was inoculated into three 500-mL flasks containing of 100 mL of the vegetative medium. The second seed cultures were incubated at 28° C. and 250 rpm on a rotary shaker for 2 days. Five mL each of the second seed culture was inoculated into thirty-five 500-mL flasks containing of 100 mL of the vegetative medium. The third seed cultures were incubated at 28° C. and 250 rpm on a rotary shaker for 2 days. Five mL each of the third seed culture was inoculated into four hundred 500-mL flasks containing 100 mL of the Production Medium A consisting of the following per liter of deionized water: starch, 10 g; yeast extract, 4 g; Hy-Soy, 4 g; ferric sulfate, 40 mg; potassium bromide, 100 mg; calcium carbonate, 1 g; and synthetic sea salt (Instant Ocean, Aquarium Systems), 30 g. The production cultures were incubated at 28° C. and 250 rpm on rotary shakers for 1 day. Approximately 2 to 3 grams of sterile Amberlite XAD-7 resin were added to the production cultures. The production cultures were further incubated at 28° C. and 250 rpm on rotary shakers for 5 days and achieved a titer of Compound II-16 of about 200 mg/L. The culture broth was filtered through cheese cloth to recover the Amberlite XAD-7 resin. The resin was extracted with 2 times 6 liters ethyl acetate followed by 1 time 1.5 liters ethyl acetate. The combined extracts were dried in vacuo. The dried extract, containing 3.8 grams the compound of Formula II-16 and lesser quantities of compounds of formulae II-20 and II-24C, was then processed for the recovery of the compounds of Formula I-7, II-16, II-20, II-24C, II-26 and II-28.

### Example 2

### Fermentation of Compounds I-7, II-16, II-17, II-20, II-24C, II-26 and II-28 Using Strain NPS21184

Strain NPS21184 was grown in a 500-mL flask containing 100 mL of vegetative medium consisting of the following per liter of deionized water: glucose, 8 g; yeast extract, 6 g; Hy-Soy, 6 g; and synthetic sea salt (Instant Ocean, Aquarium Systems), 30 g. The first seed culture was incubated at 28° C. for 3 days on a rotary shaker operating at 250 rpm. Five mL of the first seed culture was inoculated into 500-mL flask containing of 100 mL of the vegetative medium. The second seed cultures were incubated at 28° C. and 250 rpm on a rotary shaker for 2 days. Five mL each of the second seed culture was inoculated into 500-mL flask containing of 100 mL of the vegetative medium. The third seed cultures were incubated at 28° C. and 250 rpm on a rotary shaker for 2 days. Five mL each of the third seed culture was inoculated into 500-mL flask containing 100 mL of the Production Medium B consisting of the following per liter of deionized water: starch, 20 g; yeast extract, 4 g; Hy-Soy, 8 g; ferric sulfate, 40 mg; potassium bromide, 100 mg; calcium carbonate, 1 g; and synthetic sea salt (Instant Ocean, Aquarium Systems), 30 g. The production cultures were incubated at 28° C. and 250 rpm on rotary shakers for 1 day. Approximately 2 to 3 grams of sterile Amberlite XAD-7 resin were added to the production culture. The production culture was further incubated at 28° C. and 250 rpm on rotary shaker for 4 days and achieved a titer of 350-400 mg/L for Compound II-16.

Alternatively, the production of the compounds can be achieved in a 42 L fermentor system using strain NPS21184. Strain NPS21184 was grown in a 500-mL flask containing 100 mL of vegetative medium consisting of the following per liter of deionized water: glucose, 8 g; yeast extract, 6 g; Hy-Soy, 6 g; and synthetic sea salt (Instant Ocean, Aquarium Systems), 30 g. The first seed culture was incubated at 28° C. for 3 days on a rotary shaker operating at 250 rpm. Five mL of the first seed culture was inoculated into 500-mL flask containing of 100 mL of the vegetative medium. The second seed cultures were incubated at 28° C. and 250 rpm on a rotary shaker for 2 days. Twenty mL each of the second seed culture was inoculated into 2.8 L Fernbach flask containing of 400 mL of the vegetative medium. The third seed cultures were incubated at 28° C. and 250 rpm on a rotary shaker for 2 days. 1.2 L of the third seed culture was inoculated into a 42 L fermentor containing 26 L of Production Medium A. Production Medium B and Production Medium C, with the following composition, can also be used. Production Medium C consisting of the following per liter of deionized water: starch, 15 g; yeast extract 6 g; Hy-Soy, 6 g; ferric sulfate, 40 mg; potassium bromide, 100 mg; calcium carbonate, 1 g; and synthetic sea salt (Instant Ocean, Aquarium Systems), 30 g. The fermentor cultures were operated at the following parameters: temperature, 28° C.; agitation, 200 rpm; aeration, 13 L/min and back pressure, 4.5 psi. At 36 to 44 hours of the production cycle, approximately 600 grams of sterile Amberlite XAD-7 resin were added to the fermentor culture. The production culture was further incubated at the above operating parameters until day 4 of the production cycle. The aeration rate was lowered to 8 L/min. At day 5 of the production cycle, the fermentor culture achieved a titer of about 300 mg/L for Compound II-16. The culture broth was filtered through cheese cloth to recover the Amberlite XAD-7 resin. The resin was extracted with 2 times 4.5 liters ethyl acetate followed by 1 time 1.5 liters ethyl acetate. The combined extracts were dried in vacuo. The dried extract was then processed for the recovery of the Compounds of Formulae I-7, II-16, II-17, II-20, II-24C, II-26 and II-28.

### Example 3

### Purification of Compound of Formulae I-7, II-16, II-20, II-24C, II-26 and II-28

### 3A: Purification of Compound of Formulae II-16, II-20, II-24C, II-26 and II-28

The pure compounds of Formulae II-16, II-20 II-24C, II-26 and II-28 were obtained by flash chromatography followed by HPLC. Eight grams crude extract containing 3.8 grams of the compound of Formula II-16 and lesser quantities of II-20, II-24C, II-26 and II-28 was processed by flash chromatography using Biotage Flash40i system and Flash 40M cartridge (KP-Sil Silica, 32-63 μm, 90 grams). The flash chromatography was developed by the following step gradient:

1. Hexane (1 L)

2. 10% Ethyl acetate in hexane (1 L)

3. 20% Ethyl acetate in hexane, first elution (1 L)

4. 20% Ethyl acetate in hexane, second elution (1 L)

5. 20% Ethyl acetate in hexane, third elution (1 L)

6. 25% Ethyl acetate in hexane (1 L)

7. 50% Ethyl acetate in hexane (1 L)

8. Ethyl acetate (1 L)

Fractions containing the compound of Formula II-16 in greater or equal to 70% UV purity by HPLC were pooled and subject to HPLC purification, as described below, to obtain II-16, along with II-20 and II-24C, each as pure compounds

| | |
|---|---|
| Column | Phenomenex Luna 10 μm Silica |
| Dimensions | 25 cm × 21.2 mm ID |
| Flow rate | 25 mL/min |
| Detection | ELSD |
| Solvent | Gradient of 24% EtOAc/hexane for 19 min, |
| | 24% EtOAc/hexane to 100% EtOAc in 1 |
| | min, then 100% EtOAc for 4 min |

The fraction enriched in compound of Formula II-16 (described above; ˜70% pure with respect to II-16) was dissolved in acetone (60 mg/mL). Aliquots (950 μL) of this solution were injected onto a normal-phase HPLC column using the conditions described above. Compound II-16 typically eluted after 14 minutes and compounds II-24C and II-26 co-eluted as a single peak at 11 min. When parent samples containing compounds II-17, II-20 and II-28 were processed, compound II-17 eluted at 22 minutes, while II-20 and II-28 co-eluted at 23 minutes during the 100% ethyl acetate wash. Fractions containing compound II-16 and minor analogs were pooled based on composition of compounds present, and evaporated under reduced pressure on a rotary evaporator. This process yielded pure Compound A, as well as separate fractions containing minor compounds II-20, II-24C, II-26 and II-28, which were further purified as described below.

Sample containing II-24C and II-26 generated from the process described above were further separated using reversed-phase preparative HPLC as follows. The sample containing II-24C (70 mg) was dissolved in acetonitrile at a concentration of 10 mg/mL, and 500 μL was loaded on an HPLC column of dimensions 21 mm i.d. by 15 cm length containing Eclipse XDB-C18 support. The solvent gradient increased linearly from 15% acetonitrile/85% water to 100% acetonitrile over 23 minutes at a flow rate of 14.5 mL/min. The solvent composition was held at 100% acetonitrile for 3 minutes before returning to the starting solvent mixture. Compound II-26 eluted at 17.5 minutes while compound II-24C eluted at 19 minutes under these conditions.

Crystalline II-26 was obtained using a vapor diffusion method. Compound II-26 (15 mg) was dissolved in 100 μL of acetone in a 1.5 mL v-bottom HPLC vial. This vial was then placed inside a larger sealed vessel containing 1 mL of pentane. Crystals suitable for X-ray crystallography experiments were observed along the sides and bottom of the inner vial after 48 hours of incubation at 4° C. Crystallography data was collected on a Bruker SMART APEX CCD X-ray diffractometer (F(000)=2656, MO_{Kα} radiation, λ=0.71073 Å, μ=0.264 mm⁻¹, T=100 K) at the UCSD Crystallography Lab and the refinement method used was full-matrix least-squares on F². Crystal data NPI-2065: C₁₅H₂₀ClNO₄, MW=313.77, tetragonal, space group P4(1)2(1)2, a=b=11.4901(3) Å, c=46.444(2) Å, α=β=γ=90°, vol=6131.6(3) Å³, Z=16, ρ_{calcd}=1.360 g cm⁻³, crystal size, 0.30×0.15×0.07 mm³, θ range, 1.75-26.00°, 35367 reflections collected, 6025 independent reflections (Rᵢₙₜ=0.0480), final R indices (I>2σ(I)): R₁=0.0369, wR₂=0.0794, GOF=1.060.

In order to separate II-28 from II-20, a reverse-phase isocratic method was employed. Sample (69.2 mg) containing both compounds was dissolved in acetonitrile to a concentration of 10 mg/mL, and 500 μL was loaded on a reverse-phase HPLC column (ACE 5 C18-HL, 15 cm×21 mm ID) per injection. An isocratic solvent system of 27% acetonitrile/63% water at flow rate of 14.5 mL/min was used to separate compounds II-28 and II-20, which eluted after 14 and 16 minutes, respectively. Fractions containing compounds of interest were immediately evaporated under reduced pressure at room temperature on a rotary evaporator. Samples were then loaded onto a small column of silica and eluted with 10 mL of 70% hexane/30% acetone to remove additional impurities.

Samples generated from the preparative normal-phase HPLC method described above that contained II-20, but which were free of II-28 could also be triturated with 100% EtOAc to remove minor lipophilic impurities.

Compound of Formula II-16: UV (Acetonitrile/H₂O) λₘₐₓ 225(sh) nm. Low Res. Mass: m/z 314 (M+H), 336 (M+Na).

Compound of Formula II-20: UV (Acetonitrile/H₂O) λₘₐₓ 225(sh) nm. Low Res. Mass: m/z 266 (M+H); HRMS (ESI), m/z 266.1396 (M+H), Δ_{calc}=1.2 ppm.

Compound of Formula II-24C: UV (Acetonitrile/H₂O) λₘₐₓ 225(sh) nm. Low Res. Mass: m/z 328 (M+H), 350 (M+Na); HRMS (ESI), m/z 328.1309 (M+H), Δ_{calc}=−2.0 ppm, C₁₆H₂₃NO₄Cl.

Compound of Formula II-26: UV (Acetonitrile/H₂O) λₘₐₓ 225(sh) nm; HRMS (ESI), m/z 314.1158 (M+H), Δ_{calc}=−0.4 ppm, C₁₅H₂₁NO₄Cl.

Compound of Formula II-28: UV (Acetonitrile/H₂O) λₘₐₓ 225(sh) nm; HRMS (ESI), m/z 266.1388 (M+H), Δ_{calc}=−1.8 ppm, C₁₄H₂₀NO₄.

### 3B: Purification of Compound of Formula I-7

A Biotage Flash 75Li system with a Flash 75L KP-Sil cartridge was used to process the filtered crude extract (10.0 g), enriched in Compound II-16 and containing Compound of Formula I-7. The crude extract was dissolved to a concentration of 107 mg/mL in acetone and loaded directly onto the cartridge. The following solvent step gradient was then run through the cartridge at a flow rate between 235 mL/min and 250 mL/min

1. 10% EtOAc in n-Heptane (3.2 L)

2. 25% EtOAc in n-Heptane (16 L)

3. 30% EtOAc in n-Heptane (5.4 L)

Fractions enriched in Compound II-16 were pooled and concentrated by rotavapor until ˜5% of the total pooled volume of solvent remained. The solvent was removed, leaving behind the white solid.

A crystallization was then performed on the solid by dissolving the sample (4.56 g) in 1:1 acetone:n-heptane (910 mL). The solvent was slowly evaporated using a rotary evaporator until the solvent was reduced to about 43% of its original volume. The solution (supernatant) was removed and concentrated (598 mg).

The supernatant was dissolved in acetone (80 mg/mL). Aliquots (500 μL) of this solution were injected onto a normal-phase HPLC column using the conditions described above for normal phase purification of Compounds II-16, II-24C, II-26 and II-28. Compound of Formula I-7 eluted at 7.5 minutes as a pure compound.

Compound of Formula I-7: UV (Acetonitrile/H₂O) λₘₐₓ 225(sh) nm. Low Res. Mass: m/z 298 (M+H), 320 (M+Na).

### Example 4

### Fermentation of Compounds of Formulae II-17, II-18, and II-27

Strain CNB476 was grown in a 500-mL flask containing 100 mL of the first vegetative medium consisting of the following per liter of deionized water: glucose, 4 g; Bacto tryptone, 3 g; Bacto casitone, 5 g; and synthetic sea salt (Instant Ocean, Aquarium Systems), 30 g. The first seed culture was incubated at 28 C for 3 days on a rotary shaker operating at 250 rpm. Five mL of the first seed culture was inoculated into a 500-mL flask containing 100 mL of the second vegetative medium consisting of the following per liter of deionized water: starch, 10 g; yeast extract, 4 g; peptone, 2 g; ferric sulfate, 40 mg; potassium bromide, 100 mg; calcium carbonate, 1 g; and sodium bromide, 30 g. The second seed cultures were incubated at 28° C. for 7 days on a rotary shaker operating at 250 rpm. Approximately 2 to 3 gram of sterile Amberlite XAD-7 resin were added to the second seed culture. The second seed culture was further incubated at 28° C. for 2 days on a rotary shaker operating at 250 rpm. Five ml of the second seed culture was inoculated into a 500-ml flask containing 100 mL of the second vegetative medium. The third seed culture was incubated at 28° C. for 1 day on a rotary shaker operating at 250 rpm. Approximately 2 to 3 gram of sterile Amberlite XAD-7 resin were added to the third seed culture. The third seed culture was further incubated at 28° C. for 2 days on a rotary shaker operating at 250 rpm. Five ml of the third culture was inoculated into a 500-ml flask containing 100 mL of the second vegetative medium. The fourth seed culture was incubated at 28° C. for 1 day on a rotary shaker operating at 250 rpm. Approximately 2 to 3 gram of sterile Amberlite XAD-7 resin were added to the fourth seed culture. The fourth seed culture was further incubated at 28° C. for 1 day on a rotary shaker operating at 250 rpm. Five mL each of the fourth seed culture was inoculated into ten 500-mL flasks containing 100 mL of the second vegetative medium. The fifth seed cultures were incubated at 28° C. for 1 day on a rotary shaker operating at 250 rpm. Approximately 2 to 3 grams of sterile Amberlite XAD-7 resin were added to the fifth seed cultures. The fifth seed cultures were further incubated at 28° C. for 3 days on a rotary shaker operating at 250 rpm. Four mL each of the fifth seed culture was inoculated into one hundred and fifty 500-mL flasks containing 100 mL of the production medium having the same composition as the second vegetative medium. Approximately 2 to 3 grams of sterile Amberlite XAD-7 resin were also added to the production culture. The production cultures were incubated at 28° C. for 6 day on a rotary shaker operating at 250 rpm. The culture broth was filtered through cheese cloth to recover the Amberlite XAD-7 resin. The resin was extracted with 2 times 3 liters ethyl acetate followed by 1 time 1 liter ethyl acetate. The combined extracts were dried in vacuo. The dried extract, containing 0.42 g of the compound Formula II-17 and 0.16 gram the compound of Formula II-18, was then processed for the recovery of the compounds.

### Example 5

### Purification of Compounds of Formula II-17, II-18 and II-27

The pure compounds of Formula II-17 and II-18 were obtained by reversed-phase HPLC as described below:

| | |
|---|---|
| Column | ACE 5 C18-HL |
| Dimensions | 15 cm × 21 mm ID |
| Flow rate | 14.5 mL/min |
| Detection | 214 nm |
| Solvent | Gradient of 35% acetonitrile/65% |
| | H₂O to 90% acetonitrile/10% H₂O |
| | over 15 min |

Crude extract (100 mg) was dissolved in 15 mL of acetonitrile. Aliquots (900 μL) of this solution were injected onto a reversed-phase HPLC column using the conditions described above. Compounds of Formulae II-17 and II-18 eluted at 7.5 and 9 minutes, respectively. Fractions containing the pure compounds were first concentrated using nitrogen to remove organic solvent. The remaining solution was then frozen and lyophilized to dryness.

An alternative purification method for Compound II-17 and II-18 was developed for larger scale purification and involved fractionation of the crude extract on a normal phase VLC column. Under these conditions, sufficient amounts of several minor metabolites were identified, including compound II-27. The crude extract (2.4 g) was dissolved in acetone (10 mL) and this solution adsorbed onto silica gel (10 cc) by drying in vacuo. The adsorbed crude extract was loaded on a normal phase silica VLC column (250 cc silica gel, column dimensions 2.5 cm diameter by 15 cm length) and washed with a step gradient of hexane/EtOAc, increasing in the percentage of hexane in steps of 5% (100 mL solvent per step). The majority of compound II-16 eluted in the 60% hexane/40% EtOAc wash while the majority of compound II-17 eluted in the 50% hexane/50% ethyl acetate wash. Final separation of the compounds was achieved using C18 HPLC chromatography (ACE 5 μm C18-HL, 150 mm×21 mm ID) using an isocratic solvent system consisting of 35% acetonitrile/65% H₂O. Under these conditions, compound II-27 eluted at 11 minutes, compound II-17 eluted at 12.0 minutes, traces of compound A eluted at 23.5 minutes, and compound II-18 eluted at 25.5 minutes. The resulting samples were dried in vacuo using no heat to remove the aqueous solvent mixture. The spectroscopic data for these samples of compound II-16 and compound II-18 were found to be identical with those of samples prepared from earlier purification methods. The sample of compound II-18 was found to contain 8% of the lactone hydrolysis product and was further purified by washing through a normal phase silica plug (1 cm diameter by 2 cm height) and eluting using a solvent mixture of 20% EtOAc/80% Hexanes (25 mL). The resulting sample was found to contain pure compound II-18.

The fractions containing compound II-27 described above were further purified using normal phase semipreparative HPLC (Phenomenex Luna Si 10 μm, 100 Å; 250×10 mm id) using a solvent gradient increasing from 100% hexane to 100% EtOAc over 20 minutes with a flowrate of 4 mL/min. Compound II-27 eluted as a pure compound after 11.5 minutes (0.8 mg, 0.03% isolated yield from dried extract weight).

Compound of Formula II-17: UV (Acetonitrile/H₂O) λₘₐₓ 225(sh) nm. High Res. Mass (APCI): m/z 280.156 (M+H), Δ_{calc}=2.2 ppm, C₁₅H₂₂NO₄.

Compound of Formula II-18: UV (Acetonitrile/H₂O) λₘₐₓ 225(sh) nm. High Res. Mass (APCI): m/z 358.065 (M+H), Δ_{calc}=−1.9 ppm, C₁₅H₂₁NO₄Br.

Compound II-27: UV (Acetonitrile/H₂O) λₘₐₓ 225(sh) nm; MS (HR-ESI), m/z 280.1556 (M+H) Δ_{calc}=2.7 ppm (C₁₅H₂₂NO₄).

### Example 6

### Preparation of Compound of Formula II-19 from II-16

A sample of compound of Formula II-16 (250 mg) was added to an acetone solution of sodium iodide (1.5 g in 10 mL) and the resulting mixture stirred for 6 days. The solution was then filtered through a 0.45 micron syringe filter and injected directly on a normal phase silica HPLC column (Phenomenex Luna 10 μm Silica, 25 cm×21.2 mm) in 0.95 mL aliquots. The HPLC conditions for the separation of compound formula II-19 from unreacted II-16 employed an isocratic HPLC method consisting of 24% ethyl acetate and 76% hexane, in which the majority of compound II-19 eluted 2.5 minutes before compound II-16. Equivalent fractions from each of 10 injections were pooled to yield 35 mg compound II-19. Compound II-19: UV (Acetonitrile/H₂O) 225 (sh), 255 (sh) nm; ESMS, m/z 406.0 (M+H); HRMS (ESI), m/z 406.0513 [M+H]⁺, Δ_{calc}=−0.5 ppm, C₁₅H₂₁NO₄I.

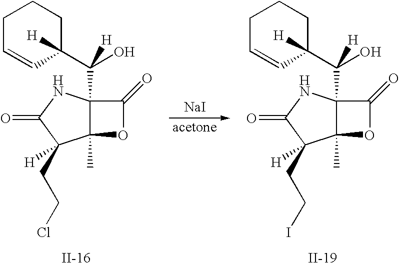

### Example 7

### Synthesis of the Compounds of Formulae II-2, II-3, and II-4

Compounds of Formulae II-2, II-3 and II-4 can be synthesized from compounds of Formulae II-16, II-17 and II-18, respectively, by catalytic hydrogenation.

### Exemplary Depiction of Synthesis

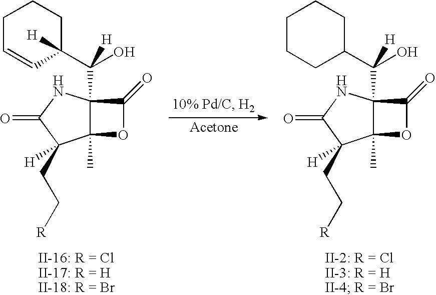

### Example 7A

### Catalytic Hydrogenation of Compound of Formula II-16

Compound of Formula II-16 (10 mg) was dissolved in acetone (5 mL) in a scintillation vial (20 mL) to which was added the 10% (w/w) Pd/C (1-2 mg) and a magnetic stirrer bar. The reaction mixture was stirred in a hydrogen atmosphere at room temperature for about 15 hours. The reaction mixture was filtered through a 3 cc silica column and washed with acetone. The filtrate was filtered again through 0.2 μm Gelman Acrodisc to remove any traces of catalyst. The solvent was evaporated off from filtrate under reduced pressure to yield the compound of Formula II-2 as a pure white powder: UV (acetonitrile/H₂O): λₘₐₓ 225 (sh) nm: m/z 316 (M+H), 338 (M+Na).

### Example 7B

### Catalytic Hydrogenation of Compound of Formula II-17

Compound of Formula II-17 (5 mg) was dissolved in acetone (3 mL) in a scintillation vial (20 mL) to which was added the 10% (w/w) Pd/C (about 1 mg) and a magnetic stirrer bar. The reaction mixture was stirred in a hydrogen atmosphere at room temperature for about 15 hours. The reaction mixture was filtered through a 0.2 μm Gelman Acrodisc to remove the catalyst. The solvent was evaporated off from filtrate to yield the compound of Formula II-3 as a white powder which was purified by normal phase HPLC using the following conditions:

| | |
|---|---|
| Column: | Phenomenex Luna 10 μm Silica |
| Dimensions: | 25 cm × 21.2 mm ID |
| Flow rate: | 14.5 mL/min |
| Detection: | ELSD |
| Solvent: | 5% to 60% EtOAc/Hex for 19 min, 60 to 100% EtOAc in 1 |
| | min, then 4 min at 100% EtOAc |

Compound of Formula II-3 eluted at 22.5 min as a pure compound: UV (acetonitrile/H₂O): λₘₐₓ 225 (sh) nm: m/z 282 (M+H), 304 (M+Na).

### Example 7C

### Catalytic Hydrogenation of Compound of Formula II-18

3.2 mg of compound of Formula I-18 was dissolved in acetone (3 mL) in a scintillation vial (20 mL) to which was added the 10% (w/w) Pd/C (about 1 mg) and a magnetic stirrer bar. The reaction mixture was stirred in hydrogen atmosphere at room temperature for about 15 hours. The reaction mixture was filtered through a 0.2 μm Gelman Acrodisc to remove the catalyst. The solvent was evaporated off from filtrate to yield the compound of Formula II-4 as a white powder which was further purified by normal phase HPLC using the following conditions:

| | |
|---|---|
| Column: | Phenomenex Luna 10 μm Silica |
| Dimensions: | 25 cm × 21.2 mm ID |
| Flow rate: | 14.5 mL/min |
| Detection: | ELSD |
| Solvent: | 5% to 80% EtOAc/Hex for 19 min, 80 to 100% EtOAc in 1 |
| | min, then 4 min at 100% EtOAc |

Compound of Formula II-4 eluted at 16.5 min as a pure compound: UV (acetonitrile/H₂O): λₘₐₓ 225 (sh) nm: m/z 360 (M+H), 382 (M+Na).

In addition, high resolution mass spectrometry data were obtained for compounds II-2, II-3, and II-4. Compound II-2: HRMS (ESI), m/z 316.1305 [M+H]⁺, Δ_{calc}=−3.5 ppm, C₁₅H₂₃NO₄Cl. Compound II-3: HRMS (ESI), m/z 282.1706 [M+H]⁺, Δ_{calc}=0.3 ppm, C₁₅H₂₄NO₄. Compound II-4: HRMS (ESI), m/z 360.0798 [M+H]⁺, Δ_{calc}=−3.4 ppm, C₁₅H₂₃NO₄Br.

### Example 8

### Synthesis of the Compounds of Formulae II-5A and II-5B

Compounds of Formula II-5A and Formula II-5B can be synthesized from compound of Formula II-16 by epoxidation with mCPBA.

Compound of Formula II-16 (101 mg, 0.32 mmole) was dissolved in methylenechloride (30 mL) in a 100 mL of round bottom flask to which was added 79 mg (0.46 mmole) of meta-chloroperbenzoic acid (mCPBA) and a magnetic stir bar. The reaction mixture was stirred at room temperature for about 18 hours. The reaction mixture was poured onto a 20 cc silica flash column and eluted with 120 mL of CH₂Cl₂, 75 mL of 1:1 ethyl acetate/hexane and finally with 40 ml of 100% ethyl acetate. The 1:1 ethyl acetate/hexane fractions yield a mixture of diastereomers of epoxyderivatives, Formula II-5A and II-5B, which were separated by normal phase HPLC using the following conditions:

| | |
|---|---|
| Column | Phenomenex Luna 10 μm Silica |
| Dimensions | 25 cm × 21.2 mm ID |
| Flow rate | 14.5 mL/min |
| Detection | ELSD |
| Solvent | 25% to 80% EtOAc/Hex over 19 |
| | min, 80 to 100% EtOAc in 1 min, |
| | then 5 min at 100% EtOAc |

Compound Formula II-5A (major product) and II-5B (minor product) eluted at 21.5 and 19 min, respectively, as pure compounds. Compound II-5B was further chromatographed on a 3 cc silica flash column to remove traces of chlorobenzoic acid reagent.

### Chemical Structures:

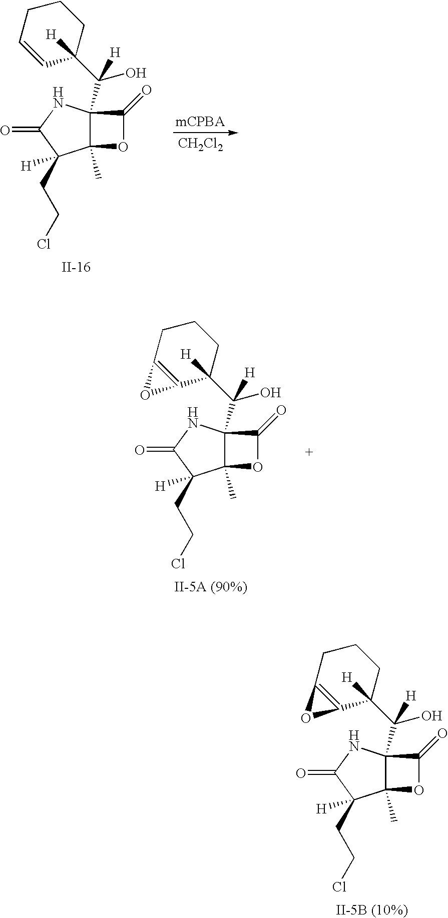

### Structural Characterization

Formula II-5A: UV (Acetonitrile/H₂O) λₘₐₓ 225 (sh) nm. Low Res. Mass: m/z 330 (M+H), 352 (M+Na); HRMS (ESI), m/z 330.1099 [M+H]⁺, Δ_{calc}=−2.9 ppm, C₁₅H₂₁NO₅Cl.

Formula II-5B: UV (Acetonitrile/H₂O) λₘₐₓ 225 (sh) nm. Low Res. Mass: m/z 330 (M+H), 352 (M+Na); HRMS (ESI), m/z 330.1105 [M+H]⁺, Δ_{calc}=−0.9 ppm, C₁₅H₂₁NO₅Cl.

### Example 9

### Synthesis of the Compounds of Formulae III-1, III-2, III-3 and III-4

### Synthesis of Diol Derivatives (Formula III-2)

Diols can be synthesized by Sharpless dihydroxylation using AD mix-α and β: AD mix-α is a premix of four reagents, K₂OsO₂(OH)₄; K₂CO₃; K₃Fe(CN)₆; (DHQ)₂-PHAL [1,4-bis(9-O-dihydroquinine)phthalazine] and AD mix-β is a premix of K₂OsO₂(OH)₄; K₂CO₃; K₃Fe(CN)₆; (DHQD)₂-PHAL [1,4-bis(9-O-dihydroquinidine)phthalazine] which are commercially available from Aldrich. The diol can also be synthesized by acid or base hydrolysis of epoxy compounds (Formula II-5A and II-5B) which may be different to that of products obtained in Sharpless dihydroxylation in their stereochemistry at carbons bearing hydroxyl groups

### Sharpless Dihydroxylation of Compounds II-16, II-17 and II-18

Any of the compounds of Formulae II-16, II-17 and II-18 can be used as the starting compound. In the example below, compound of Formula II-16 is used. The starting compound is dissolved in t-butanol/water in a round bottom flask to which is added AD mix-α or β and a magnetic stir bar. The reaction is monitored by silica TLC as well as mass spectrometer. The pure diols are obtained by usual workup and purification by flash chromatography or HPLC. The structures are confirmed by NMR spectroscopy and mass spectrometry. In this method both hydroxyl groups are on same side.

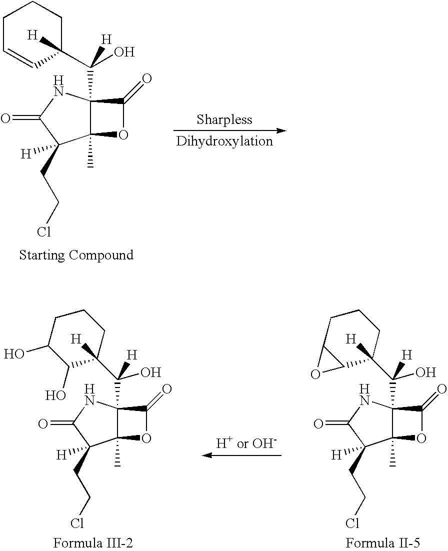

### Nucleophilic Ring Opening of Epoxy Compounds (II-5):

The epoxy ring is opened with various nucleophiles like NaCN, NaN₃, NaOAc, HBr, HCl, etc. to create various substituents on the cyclohexane ring, including a hydroxyl substituent.

### Examples:

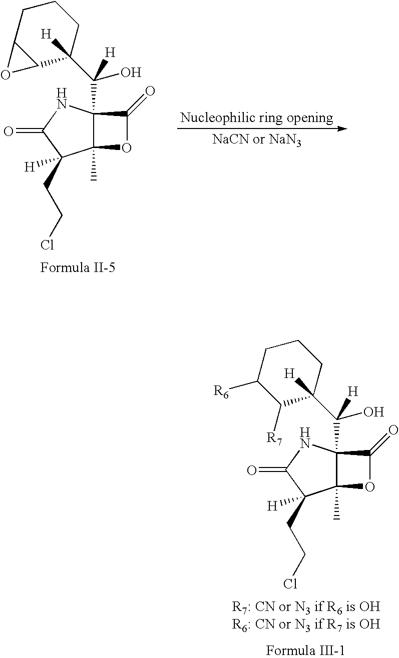

The epoxy is opened with HCl to make Formula III-3:

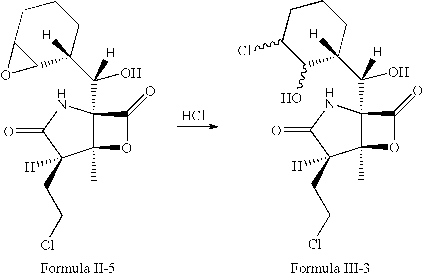

Compound of Formula II-5A (3.3 mg) was dissolved in acetonitrile (0.5 mL) in a 1 dram vial to which was added 5% HCl (500 μL) and a magnetic stir bar. The reaction mixture was stirred at room temperature for about an hour. The reaction was monitored by mass spectrometry. The reaction mixture was directly injected on normal phase HPLC to obtain compound of Formula III-3C as a pure compound without any work up. The HPLC conditions used for the purification were as follows: Phenomenex Luna 10 μm Silica column (25 cm×21.2 mm ID) with a solvent gradient of 25% to 80% EtOAc/Hex over 19 min, 80 to 100% EtOAc in 1 min, then 5 min at 100% EtOAc at a flow rate of 14.5 mL/min. An ELSD was used to monitor the purification process. Compound of Formula III-3C eluted at about 18 min (2.2 mg). Compound of Formula III-3C: UV (Acetonitrile/H₂O) λₘₐₓ 225 (sh) nm; ESMS, m/z 366 (M+H), 388 (M+Na); HRMS (ESI), m/z 366.0875 [M+H]⁺, Δ_{calc}=0.0 ppm, C₁₅H₂₂NO₅Cl₂. The stereochemistry of the compound of Formula III-3C was determined based on coupling constants observed in the cyclohexane ring in 1:1 C₆D₆/DMSO-d₆.

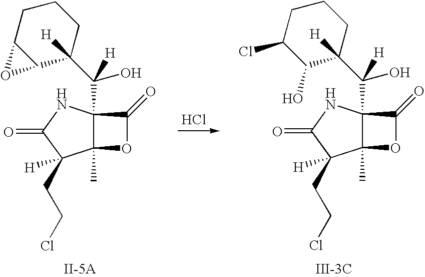

Reductive ring opening of epoxides (II-5): The compound of Formula is treated with metalhydrides like BH₃-THF complex to make compound of Formula III-4.

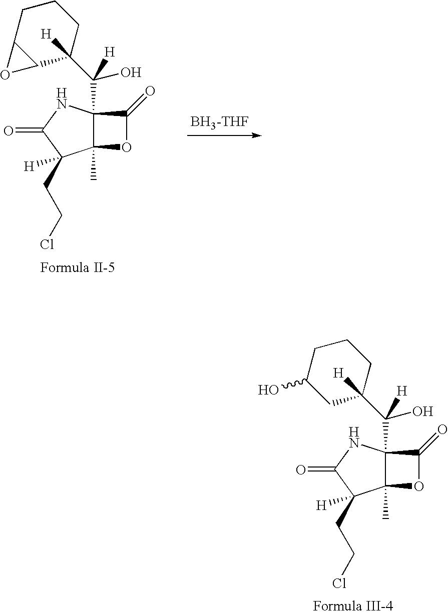

### Example 10

### Synthesis of the Compounds of Formulae II-13C and II-8C

Compound of Formula II-16 (30 mg) was dissolved in CH₂Cl₂ (6 mL) in a scintillation vial (20 mL) to which Dess-Martin Periodinane (122 mg) and a magnetic stir bar were added. The reaction mixture was stirred at room temperature for about 2 hours. The progress of the reaction was monitored by TLC (Hex:EtOAc, 6:4) and analytical HPLC. From the reaction mixture, the solvent volume was reduced to one third, absorbed on silica gel, poured on top of a 20 cc silica flash column and eluted in 20 mL fractions using a gradient of Hexane/EtOAc from 10 to 100%. The fraction eluted with 30% EtOAc in Hexane contained a mixture of rotamers of Formula II-13C in a ratio of 1.5:8.5. The mixture was further purified by normal phase HPLC using the Phenomenex Luna 10 μm Silica column (25 cm×21.2 mm ID) with a solvent gradient of 25% to 80% EtOAc/Hex over 19 min, 80 to 100% EtOAc over 1 min, holding at 100% EtOAc for 5 min, at a flow rate of 14.5 mL/min. An ELSD was used to monitor the purification process. Compound of Formula II-13C eluted at 13.0 and 13.2 mins as a mixture of rotamers with in a ratio of 1.5:8.5 (7 mg). Formula II-13C: UV (Acetonitrile/H₂O) λₘₐₓ 226 (sh) & 300 (sh) nm; ESMS, m/z 312 (M+H)⁺, 334 (M+Na)⁺; HRMS (ESI), m/z 312.1017 [M+H]⁺, Δ_{calc}=4.5 ppm, C₁₅H₁₉NO₄Cl.

The rotamer mixture of Formula II-13C (4 mg) was dissolved in acetone (1 mL) in a scintillation vial (20 mL) to which a catalytic amount (0.5 mg) of 10% (w/w) Pd/C and a magnetic stir bar were added. The reaction mixture was stirred in a hydrogen atmosphere at room temperature for about 15 hours. The reaction mixture was filtered through a 0.2 μm Gelman Acrodisc to remove the catalyst. The solvent was evaporated from the filtrate to yield compound of Formula II-8C as a colorless gum which was further purified by normal phase HPLC using a Phenomenex Luna 10 μm Silica column (25 cm×21.2 mm ID) with a solvent gradient of 25% to 80% EtOAc/Hex over 19 min, 80 to 100% EtOAc over 1 min, holding at 100% EtOAc for 5 min, at a flow rate of 14.5 mL/min. An ELSD was used to monitor the purification process. Compound of Formula II-8C (1 mg) eluted at 13.5 min as a pure compound. Formula II-8C: UV (Acetonitrile/H₂O) λₘₐₓ 225 (sh) nm; ESMS, m/z 314 (M+H)⁺, 336 (M+Na)⁺; HRMS (ESI), m/z 314.1149 [M+H]⁺, Δ_{calc}=3.3 ppm, C₁₅H₂₁NO₄Cl.

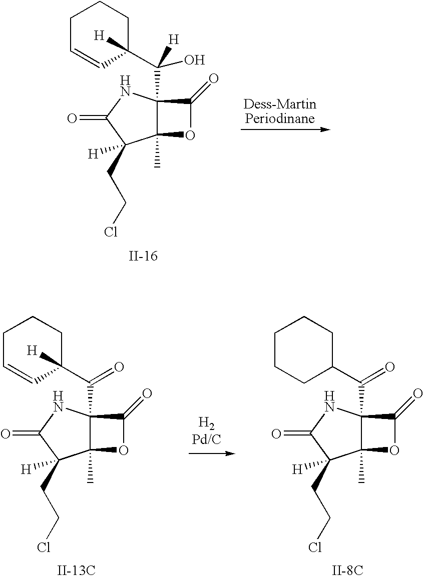

### Example 11

### Synthesis of the Compound of Formula II-25 from II-13C

The rotamer mixture of Formula II-13C (5 mg) was dissolved in dimethoxy ethane (monoglyme; 1.5 mL) in a scintillation vial (20 mL) to which water (15 μL (1% of the final solution concentration)) and a magnetic stir bar were added. The above solution was cooled to −78° C. on a dry ice-acetone bath, and a sodium borohydride solution (3.7 mg of NaBH₄ in 0.5 mL of monoglyme (created to allow for slow addition)) was added drop-wise. The reaction mixture was stirred at −78° C. for about 14 minutes. The reaction mixture was acidified using 2 mL of 4% HCl solution in water and extracted with CH₂Cl₂. The organic layer was evaporated to yield mixture of compound of formulae II-25 and II-16 in a 9.5:0.5 ratio as a white solid, which was further purified by normal phase HPLC using a Phenomenex Luna 10 μm Silica column (25 cm×21.2 mm ID). The mobile phase was 24% EtOAc/76% Hexane, which was held isocratic for 19 min, followed by a linear gradient of 24% to 100% EtOAc over 1 min, and held at 100% EtOAc for 3 min; the flow rate was 25 mL/min. An ELSD was used to monitor the purification process. Compound of formula II-25 (1.5 mg) eluted at 11.64 min as a pure compound. Compound of Formula II-25: UV (Acetonitrile/H₂O) λₘₐₓ 225 (sh) nm; ESMS, m/z 314 (M+H)⁺, 336 (M+Na)⁺; HRMS (ESI), m/z 314.1154 [M+H]⁺, Δ_{calc}=−0.6 ppm, C₁₅H₂₁NO₄Cl.

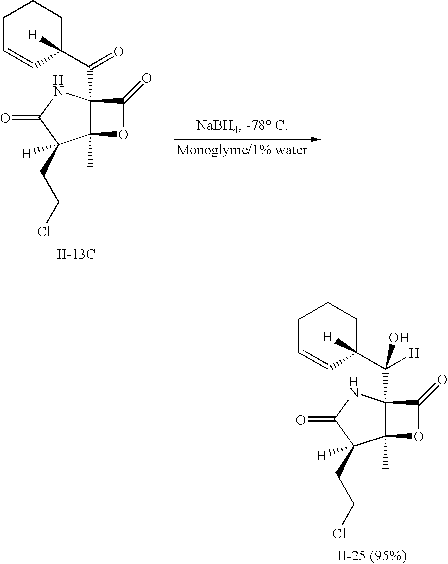

### Example 12

### Synthesis of the Compounds of Formulae II-31, II-32 and II-49 from II-13C; and Compounds of Formulae II-33, II-34, II-35 and II-36 from II-31 and II-32

A rotamer mixture of the Compound of Formula II-13C (20 mg) was dissolved in acetone (4 mL) in a scintillation vial (20 mL) to which a catalytic amount (3 mg) of 10% (w/w) Pd/C and a magnetic stir bar were added. The reaction mixture was stirred at room temperature for about 15 hours. The reaction mixture was filtered through a 0.2 μm Gelman Acrodisc to remove the catalyst. The solvent was evaporated from the filtrate to yield a mixture of diastereomers of hydroxy derivatives of Formulae II-31 and II-32 (1:1) and a minor compound II-49, which were separated by reversed phase HPLC using Ace 5 μm C18 column (150 mm×22 mm ID) with a solvent gradient of 90% to 30% H₂O/acetonitrile over 15 min, 70 to 100% acetonitrile over 5 min, holding at 100% acetonitrile for 4 min, at a flow rate of 14.5 mL/min. A diode array detector was used to monitor the purification process. Compound II-31 (2 mg), II-32 (2 mg) and II-49 (0.2 mg) eluted at 10.6, 10.8 and 11.54 min, respectively, as pure compounds. II-31: UV (Acetonitrile/H₂O) λₘₐₓ 250 (sh) nm; ESMS m/z 328.1 (M+H)⁺& 350.0 (M+Na)⁺. II-32: UV (Acetonitrile/H₂O) λₘₐₓ 250 (sh) nm; ESMS, m/z 328.1 (M+H)⁺& 350.0 (M+Na)⁺. II-49: UV (Acetonitrile/H₂O) λₘₐₓ 250 (sh) and 320 nm; ESMS, m/z 326.0 (M+H)⁺, 343.1 (M+H₂O)⁺& 348.0 (M+Na)⁺.

In an alternate method, compounds II-31, II-32 and II-49 were separated by normal phase HPLC using Phenomenex Luna 10 μm Silica column (25 cm×21.2 mm ID) with a solvent gradient of 10% to 100% Hexane/EtOAc over 24 min, holding at 100% EtOAc for 3 min, at a flow rate of 14.5 mL/min. ELSD was used to monitor the purification process.

The ketone of the compounds of formula II-31 and II-32 can be reduced by using sodium borohydride at 0 to −10° C. in monoglyme solvent for about 14 minutes. The reaction mixture can be acidified using 4% HCl solution in water and extracted with CH₂Cl₂. The organic layer can be evaporated to yield the mixtures of compounds of formulae II-33, II-34, II-35 and II-36 which can be separated by chromatographic methods.

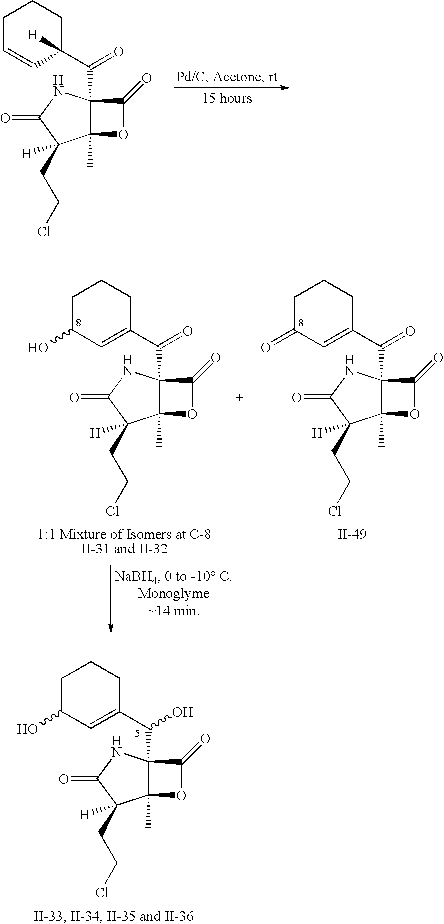

### Example 13

### Synthesis of the Compound of Formulae II-21 from II-19

Acetone (7.5 mL) was vigorously mixed with 5 N NaOH (3 mL) and the resulting mixture evaporated to a minimum volume in vacuo. A sample of 100 μL of this solution was mixed with compound of Formula II-19 (6.2 mg) in acetone (1 mL) and the resulting biphasic mixture vortexed for 2 minutes. The reaction solution was immediately subjected to preparative C18 HPLC. Conditions for the purification involved a linear gradient if 10% acetonitrile/90% water to 90% acetonitrile/10% water over 17 minutes using an Ace 5 μm C18 HPLC column of dimensions 22 mm id by 150 mm length. Compound of Formula II-21 eluted at 9.1 minutes under these conditions to yield 0.55 mg compound. Compound of Formula II-21: UV (Acetonitrile/H₂O) 225 (sh), ESMS, m/z 296.1 (M+H).

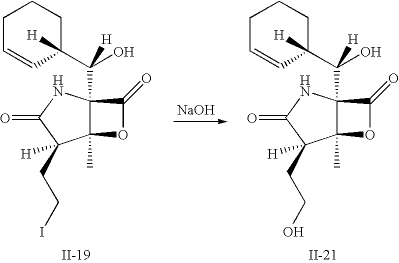

### Example 14

### Synthesis of the Compound of Formulae II-22 from II-19

A sample of 60 mg sodium propionate was added to a solution of compound of Formula II-19 (5.3 mg) in DMSO (1 mL) and the mixture sonicated for 5 minutes, though the sodium propionate did not completely dissolve. After 45 minutes, the solution was filtered through a 0.45 μm syringe filter and purified directly using HPLC. Conditions for the purification involved a linear gradient if 10% acetonitrile/90% water to 90% acetonitrile/10% water over 17 minutes using an Ace 5 μm C18 HPLC column of dimensions 22 mm id by 150 mm length. Under these conditions, compound of Formula II-22 eluted at 12.3 minutes to yield 0.7 mg compound (15% isolated yield). UV (Acetonitrile/H₂O) 225 (sh), ESMS, m/z 352.2 (M+H); HRMS (ESI), m/z 352.1762 [M+H]⁺, Δ_{calc}=0.6 ppm, C₁₈H₂₆NO₆.

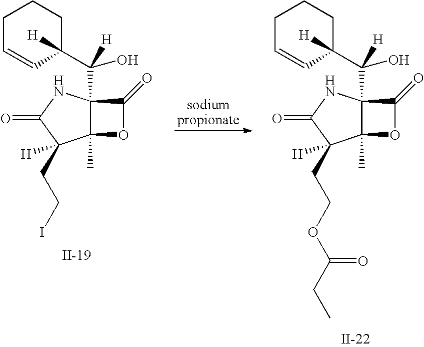

### Example 15

### Synthesis of the Compound of Formula II-29 from II-19

A sample of NaN₃ (80 mg) was dissolved in DMSO (1 mL) and transferred to a vial containing Compound II-19 (6.2 mg) which was contaminated with approximately 10% Compound II-16. The solution was incubated at room temperature for 1 hr prior to purification on C18 HPLC (ACE 5 μm C18-HL, 150 mm×21 mm ID) using a solvent gradient of 10% acetonitrile/90% H₂O to 90% acetonitrile/10% H₂O over 17 minutes. Using this method, the desired azido derivative II-29 co-eluted with Compound II-16 contaminant at 12.5 minutes (4.2 mg, 85% yield). A 2.4 mg portion of compound II-29 was further purified using additional C18 HPLC chromatography (ACE 5 μm C18-HL, 150 mm×21 mm ID) using an isocratic solvent gradient consisting of 35% acetonitrile/65% H₂O. Under these conditions compound II-29 eluted after 20 minutes, while Compound II-16 eluted after 21.5 minutes. The resulting sample consisted of 1.1 mg Compound II-29 was used for characterization in biological assays.

Compound II-29: UV (Acetonitrile/H₂O) 225 (sh), ESMS, m/z 321.1 (M+H).

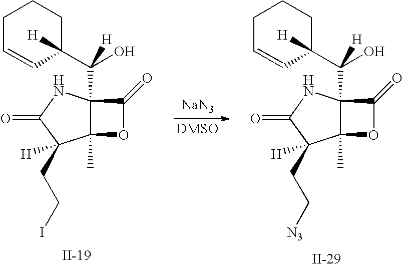

### Example 16

### Synthesis of the Compounds of Formulae II-37 and II-38 from II-19

The compounds of Formulae II-37 and II-38 can be prepared from the compound of Formula II-19 by cyano-de-halogenation or thiocyanato-de-halogenation, respectively. Compound II-19 can be treated with NaCN or KCN to obtain compound II-37. Alternatively, Compound II-19 can be treated with NaSCN or KSCN to obtain compound II-38.

Synthesis of the Compound of Formula II-38 from II-19:

The compound of formula II-19 (10.6 mg, 0.0262 mmol) was dissolved in 1.5 mL of acetone in a scintillation vial (20 mL) to which sodium thiocyanate (10.0 mg, 0.123 mmol), triethylamine (5 μL, 0.036 mmol) and a magnetic stir bar were added. The reaction mixture was stirred at room temperature for 72 hours. The reaction mixture was concentrated in vacuo to yield the compound II-38. Compound II-38 was purified by normal phase HPLC using a Phenomenex Luna 10 μm Silica column (25 cm×21.2 mm ID) with a solvent gradient of 0 to 95% H₂O/acetonitrile over 21 min, at a flow rate of 14.5 mL/min. Diode array detector was used to monitor the purification process. Compound II-38 (3.0 mg, 34% yield) eluted at 18.0 min as a pure compound. II-38: UV Acetonitrile/H₂O λₘₐₓ 203 (sh) nm; ESMS m/z 337.1 (M+H)⁺& 359.1 (M+Na)⁺.

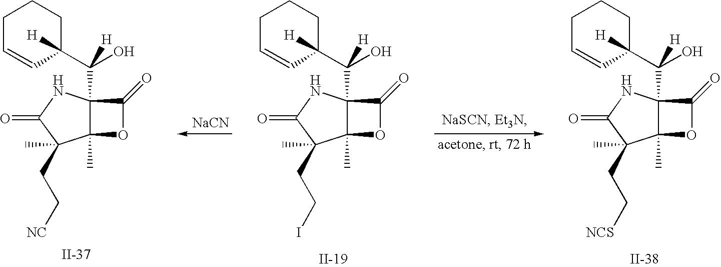

### Example 17

### Synthesis of the Compound of Formula II-39 from II-19

Thiols and thioethers of the Formula II-39 can be formed by dehalogenation of the compound of Formula II-19. Thiols (R═H) can be formed by treatment of Compound II-19 with NaSH, for example, while thioethers (R=alkyl) can be formed by treatment of Compound II-19 with salts of thiols, or alternatively, by treatment with thiols themselves by running the reaction in benzene in the presence of DBU.

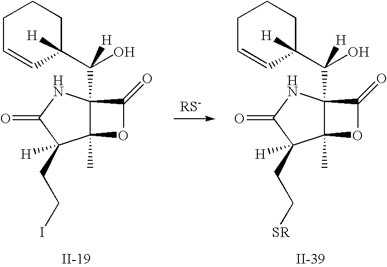

### Example 18

### Synthesis of the Compound of Formula II-40 from II-39

Sulfoxides (n=1) and sulfones (n=2) of the Formula II-40 can be formed by oxidation of thioethers of the Formula II-39, for example, with hydrogen peroxide or other oxidizing agents.

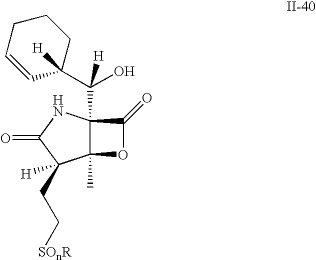

### Example 19

### Synthesis of the Compound of Formula II-41 from II-21

The compound of the Formula II-41 can be prepared by treatment of the compound of Formula II-21 (or a protected derivative of II-21, where the C-5 alcohol or lactam NH are protected, for example) with methyl sulfonyl chloride (mesyl chloride) in pyridine, for example, or by treatment with mesyl chloride in the presence of triethylaminde. Other sulfonate esters can be similarly prepared.

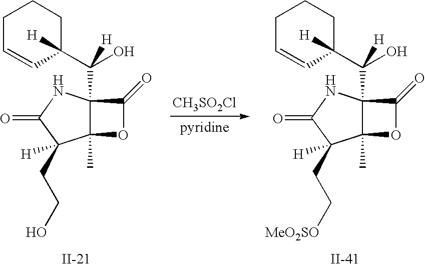

### Example 20

### Synthesis of the Compound of Formula II-46 from II-19 or II-41

The alkene of the Formula II-46 can be prepared by dehydroiodination of the compound of Formula II-19, or by hydro-mesyloxy elimination of the compound of Formula II-41, for example, by treatment with base.

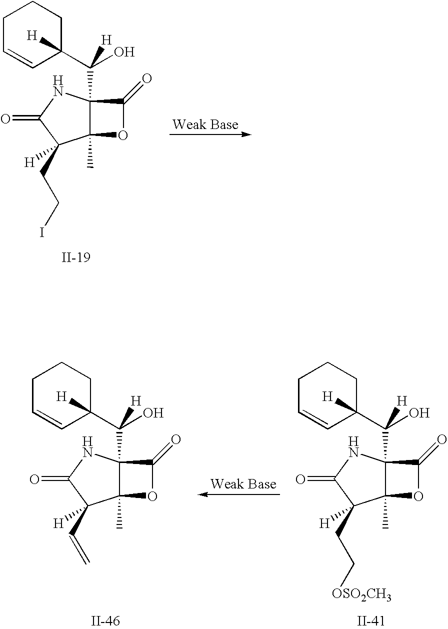

### Example 21

### Synthesis of the Compound of Formula II-42A

Synthesis of boronic acids or esters, for example, the compound of the Formula II-42A, can be achieved as outlined in the retrosynthetic scheme below. Hydroboration of the alkene of Formula II-46 gives the corresponding alkyl borane, which can be converted to the corresponding boronic acid or ester, for example, the compound of the Formula II-42A.

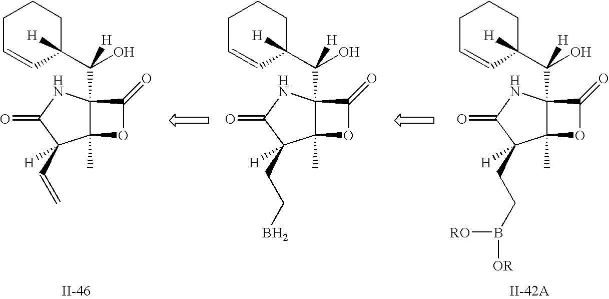

### Example 22

### Synthesis of the Compound of Formula II-43A

The compound of the Formula II-43A can be prepared by treatment of the compound of Formula II-19 with triphenyl phosphine to make a phosphorus ylide, which can be treated with various aldehydes, for example, glyoxylic acid methyl ester, to make Formula II-43A.

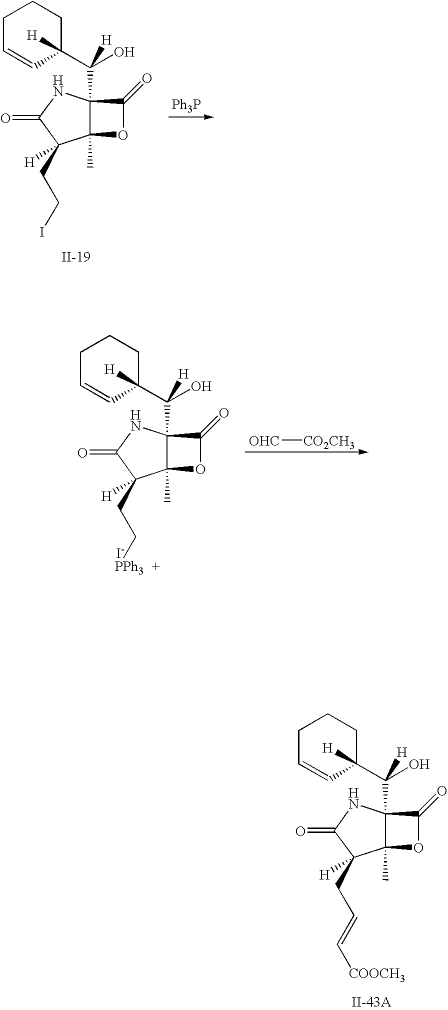

### Example 23

### Synthesis of the Compound of Formula II-30 from II-19

A portion of CuI (100 mg) was placed in a 25 mL pear bottom flask and flushed with argon gas for 30 minutes. Argon gas flow was maintained through the flask throughout the course of the reaction. The vessel was cooled to −78° C. prior to addition of dry THF (5 mL) followed by the immediate dropwise addition of a solution of methyllithium in dry THF (5.0 mL, 1.6 M) with vigorous stirring. A solution of Compound II-19 in dry THF (12 mg Compound II-19, 1 mL THF) was added slowly to the clear dialkylcuprate solution and the resulting mixture stirred at −78° C. for 1 hr. The reaction was quenched by washing the THF solution through a plug of silica gel (1 cm diameter by 2 cm length) along with further washing using a solution of 50% EtOAc/50% hexanes (50 mL). The combined silica plug washes were dried in vacuo and subjected to further C18 HPLC purification in 2 injections (ACE 5 μm C18-HL, 150 mm×21 mm ID) using an isocratic solvent gradient consisting of 35% acetonitrile/65% H₂O. Compound II-30 eluted under these conditions at 23.5 minutes and yielded 2.4 mg material (27% isolated yield) at 90.8% purity as measured by analytical HPLC. An alternative normal phase purification method can be utilized using Phenomenex Luna 10 μm Silica column (25 cm×21.2 mm ID) with a solvent gradient consisting of 100% hexanes/ethyl acetate to 0% hexanes over 20 minutes. Compound II-30 eluted under these conditions at 16.5 minutes and yielded 3.0 mg material (41% isolated yield) at 97.1% purity as measured by analytical HPLC.

Compound II-30: UV (Acetonitrile/H₂O) 225 (sh), ESMS, m/z 294.1 (M+H); HRMS (ESI), m/z 294.1696 [M+H]⁺, Δ_{calc}=−3.2 ppm, C₁₆H₂₄NO₄.

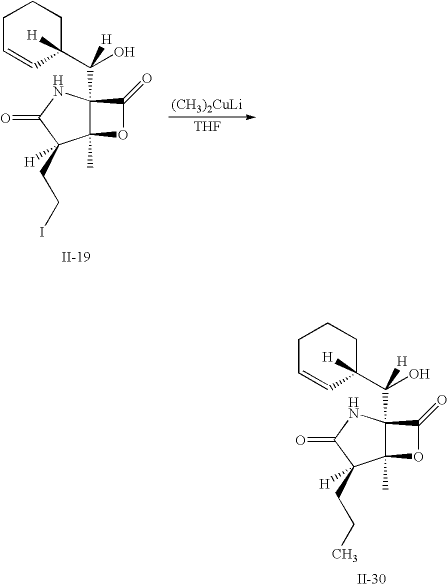

Compound II-30 can also be obtained by saline fermentation of strain CNB476. In one example, CNB476 was transferred to 500-mL flasks containing 100 mL production medium consisting of the following per liter of deionized water: starch, 10 g; yeast extract, 4 g; Hy-Soy, 4 g; ferric sulfate, 40 mg; potassium bromide, 100 mg; calcium carbonate, 1 g; and synthetic sea salt, 30 g. The production cultures were incubated at 28° C. and 250 rpm for 1 day. Approximately 2 g of sterile Amberlite XAD-7 resin was added to the production cultures. The production cultures were further incubated for 5 days. The resin was recovered from the broth and extracted with ethyl acetate. The extract was dried in vacuo. The dried extract (8 g) was then processed for the recovery of Compound II-30.

The crude extract was processed by flash chromatography using a Biotage Flash system. The flash chromatography was developed by the following step gradient: i) Hexanes (1 L); ii) 10% EtOAc in hexanes (1 L); iii) 20% EtOAc in hexanes, first elution (1 L); iv) 20% EtOAc in hexanes, second elution (1 L); v) 20% EtOAc in hexanes, third elution (1 L); yl) 25% EtOAc in hexanes (1 L); vii) 50% EtOAc in hexanes (1 L); viii) EtOAc (1 L). Fractions containing Compound II-30 was further purified by normal phase HPLC using an isocratic solvent system of 24% EtOAc/hexanes followed by a 100% EtOAc. Compound II-30 eluted 22 minutes into the isocratic portion of the run.

Fractions enriched in Compound II-30 were further processed by normal phase HPLC using a 27 minute linear gradient from 15% hexanes/85% EtOAc to 100% EtOAc. Compound II-30 eluted after 15 min.

### Example 24

### Synthesis of the Compound of Formulae II-44 from II-16

The compound of Formula II-16 (30 mg, 0.096 mmol) was dissolved in CH₂Cl₂ (9 mL) in a scintillation vial (20 mL) to which triethylamine (40 μL, 0.29 mmol), methyl-3-mercapto propionate (thiol, 250 μL) and a magnetic stir bar were added. The reaction mixture was stirred at room temperature for about 4 hours. The solvent was evaporated from the reaction mixture to yield a mixture of compound of Formulae II-44, which was separated by reversed phase HPLC using Ace 5 μm C18 column (150 mm×22 mm ID) with a solvent gradient of 35% to 90% H₂O/acetonitrile over 17 min, 90 to 100% acetonitrile overt min, holding at 100% acetonitrile for 1 min, at a flow rate of 14.5 mL/min. Diode array detector was used to monitor the purification process. Compound II-44 (20 mg) eluted at 11.68 min as a pure compound. Compound II-44: UV (Acetonitrile/H₂O) λₘₐₓ 240 (sh) nm; ESMS m/z 434.0 (M+H)⁺& 456.0 (M+Na)⁺.

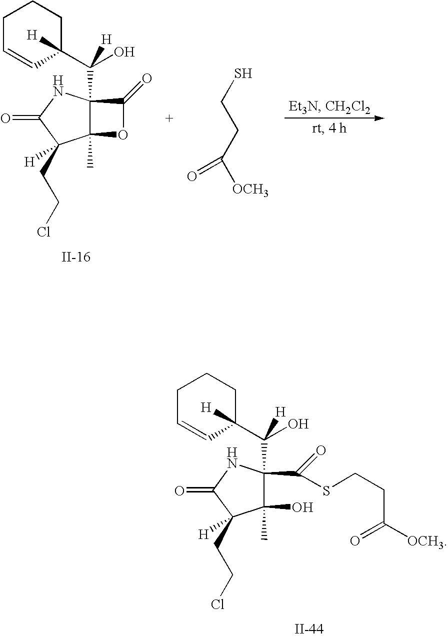

### Example 25

### Oxidation of Secondary Hydroxyl Group in Compounds of Formulae II-16, II-17 and II-18 and Reaction with Hydroxy or Methoxy Amines

Any of the compounds of Formulae II-16, II-17 and II-18 can be used as the starting compound. The secondary hydroxyl group in the starting compound is oxidized using either of the following reagents: pyridinium dichromate (PDC), pyridinium chlorochromate (PCC), Dess-Martin periodinane or oxalyl chloride (Swern oxidation) (Ref: Organic Syntheses, collective volumes I-VIII). Preferably, Dess-Martin periodinane can be used as a reagent for this reaction. (Ref: Fenteany G. et al. Science, 1995, 268, 726-73). The resulting keto compound is treated with hydroxylamine or methoxy amine to generate oximes.

### Examples:

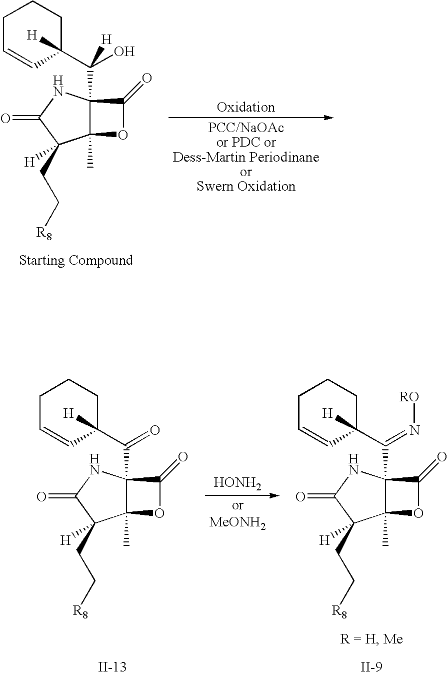

### Example 26

### Reductive Amination of Keto-Derivative

The keto derivatives, for example Formula II-8 and II-13, are treated with sodium cyanoborohydride (NaBH₃CN) in the presence of various bases to yield amine derivatives of the starting compounds which are subsequently hydrogenated with 10% Pd/C, H₂ to reduce the double bond in the cyclohexene ring.

### Example:

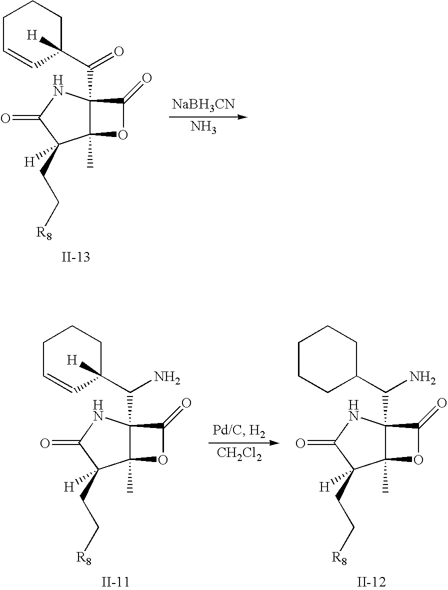

### Example 27

### Cyclohexene Ring Opening

Any compound of Formulae II-16, II-17 and II-18 can be used as a starting compound. The Starting Compounds can be protected, for example, at the alcohol and/or at the lactam nitrogen positions, and treated with OSO₄ and NaIO₄ in THF-H₂O solution to yield dial derivatives which are reduced to the alcohol with NaBH₄. The protecting groups can be removed at the appropriate stage of the reaction sequence to produce II-7 or II-6.

### Example:

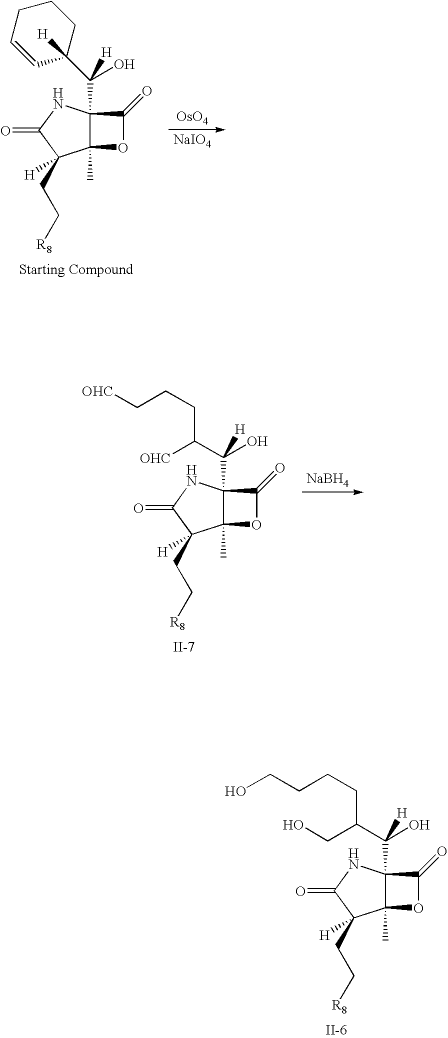

### Example 28

### Dehydration of Alcohol Followed by Aldehyde Formation at Lactone-Lactam Ring Junction

A starting compound of any of Formulae II-16, II-17 or II-18 is dehydrated, for example, by treatment with mesylchloride in the presence of base, or, for example, by treatment with Burgess reagent or other dehydrating agents. The resulting dehydrated compound is treated with OsO₄, followed by NaIO₄, or alternatively by ozonolysis, to yield an aldehyde group at the lactone-lactam ring junction.

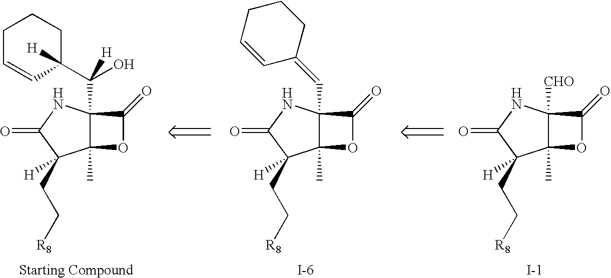

### Example 29

### Oxidation of the Cyclohexene Ring to Produce Cyclohexadienes or a Phenyl Ring

A Starting Compound, such as the ketone of Formula II-13C, is treated with Pd/C to produce a cyclohexadiene derivative. The new double bond can be at any position of the cyclohexene ring. The ketone can be reduced, for example, with sodium borohydride, to obtain the corresponding secondary alcohol(s). Alternatively, the cyclohexadiene derivative can be further treated, for example with DDQ, to aromatize the ring to a phenyl group. Similarly, the ketone can be reduced, for example, with sodium borohydride, to obtain the corresponding secondary alcohol(s).

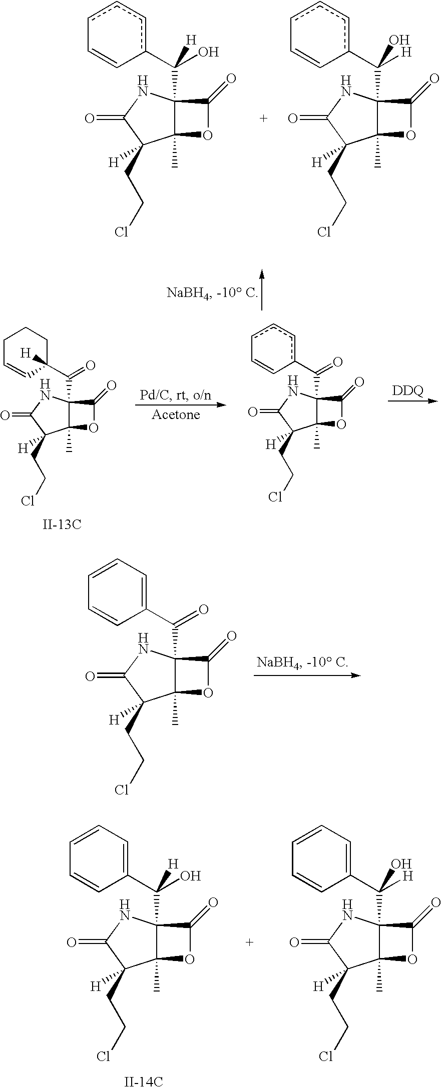

### Example 30

### Various Reactions on Aldehyde Derivatives I-1

Wittig reactions are performed on the aldehyde group using various phosphorus ylides [e.g., (triphenylphosphoranylidene)ethane] to yield an olefin. The double bond in the side chain is reduced by catalytic hydrogenation.

### Example:

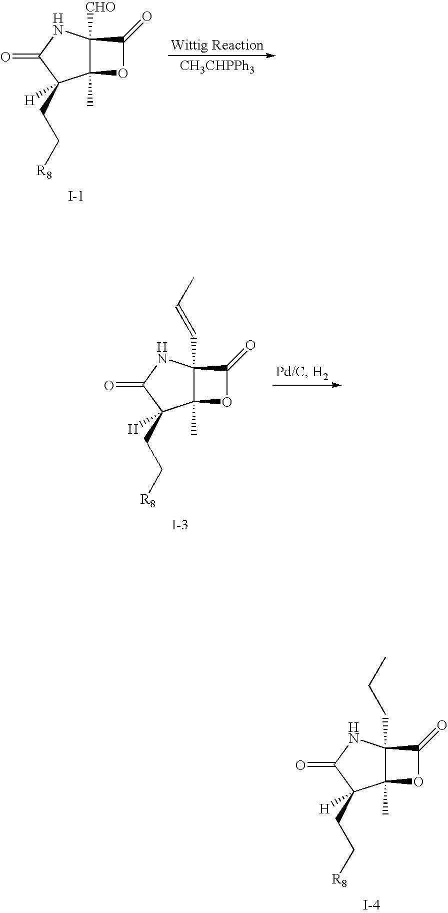

Reductive amination is performed on the aldehyde group using various bases (eg. NH₃) and sodium cyanoborohydride to yield amine derivatives. Alternatively, the aldehyde is reduced with NaBH₄ to form alcohols in the side chain.

### Example:

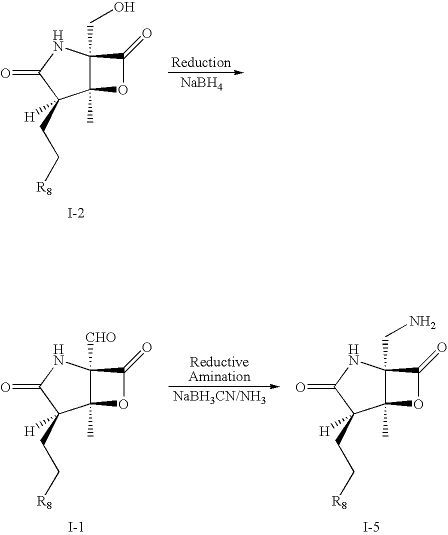

Organometallic addition reactions to the aldehyde carbonyl can be performed to yield various substituted secondary alcohols.

### Examples:

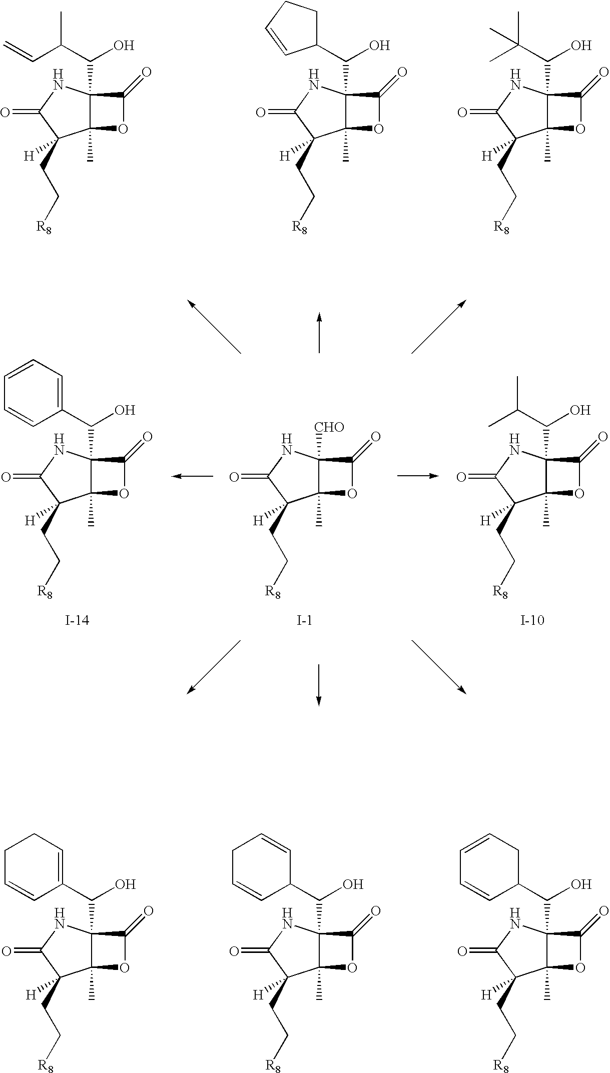

### Example 31

### Synthesis of the Compound of Formulae II-48 from II-16

The compound of Formula II-16 (15 mg, 0.048 mmol) was dissolved in 1:1 ratio of acetonitrile/DMSO (8 mL) in a scintillation vial (20 mL) to which triethylamine (40 μL, 0.29 mmol), Glutathione (44.2 mg, 0.144 mmol) and a magnetic stir bar were added. The reaction mixture was stirred at room temperature for about 3 hours. The solvent was evaporated from the reaction mixture to yield the compound of Formula II-48, which was purified by reversed phase HPLC using Ace 5 μm C18 column (150 mm×22 mm ID) with a solvent gradient of 10% to 70% H₂O/acetonitrile over 15 min, 70 to 100% acetonitrile over 5 min, holding at 100% acetonitrile for 4 min, at a flow rate of 14.5 mL/min. Diode array detector was used to monitor the purification process. Compound II-48 (10 mg) eluted as a pure compound at 8.255 min. Compound II-48: UV (Acetonitrile/H₂O) λₘₐₓ 235 (sh) nm; ESMS m/z 621.0 (M+H)⁺.

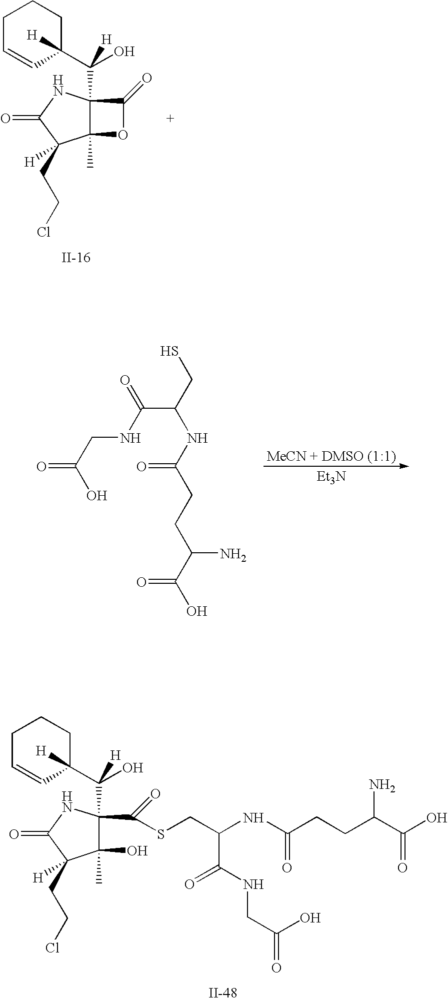

### Example 32

### Synthesis of the Compound of Formula II-50 from II-16

The compound of Formula II-16 (10 mg, 0.032 mmol) was dissolved in CH₂Cl₂ (9 mL) in scintillation vial (20 mL) to which triethylamine (26.5 μL, 0.192 mmol), N-Acetyl-L-Cysteine methyl ester (17 mg, 0.096 mmol) and a magnetic stir bar were added. The reaction mixture was stirred at room temperature for about 4 hours. The solvent was evaporated from the reaction mixture to yield the mixture of compound of Formulae II-50, which was further purified by normal phase HPLC using Phenomenex Luna 10 μm Silica column (25 cm×21.2 mm ID) with a solvent gradient of 10% to 100% Hexane/EtOAc over 24 min, holding at 100% EtOAc for 3 min, at a flow rate of 14.5 mL/min. ELSD was used to monitor the purification process. Compound II-50 (2 mg) was eluted at 10.39 min as a pure compounds. Compound II-50: UV (Acetonitrile/H₂O) λₘₐₓ 230 (sh) nm; ESMS m/z 491.1 (M+H)⁺& 513.0 (M+Na)⁺.

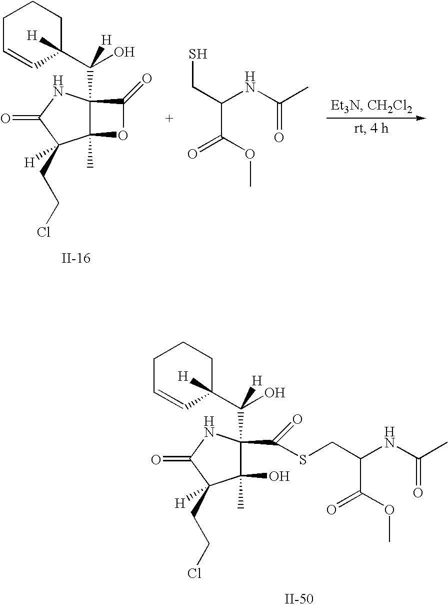

### Example 33

### Salinosporamide A (II-16) Inhibits Chymotrypsin-Like Activity of Rabbit Muscle 20S Proteasomes

The effect of Salinosporamide A (II-16) on proteasomes was examined using a commercially available kit from Calbiochem (catalog no. 539158), which uses a fluorogenic peptide substrate to measure the activity of rabbit muscle 20S proteasomes (Calbiochem 20S Proteasome Kit). This peptide substrate is specific for the chymotrypsin-like enzyme activity of the proteasome.

Omuralide was prepared as a 10 mM stock in DMSO and stored in 5 μL aliquots at −80° C. Salinosporamide A was prepared as a 25.5 mM solution in DMSO and stored in aliquots at −80° C. The assay measures the hydrolysis of Suc-LLVY-AMC into Suc-LLVY and AMC. The released coumarin (AMC) was measured fluorometrically by using λₑₓ=390 nm and λₑₘ=460 nm. The assays were performed in a microtiter plate (Corning 3904), and followed kinetically with measurements every five minutes. The instrument used was a Thermo Lab Systems Fluoroskan, with the incubation chamber set to 37° C. The assays were performed according to the manufacturer's protocol, with the following changes. The proteasome was activated as described with SDS, and held on ice prior to the assay. Salinosporamide A and Omuralide were serially diluted in assay buffer to make an 8-point dose-response curve. Ten microliters of each dose were added in triplicate to the assay plate, and 190 μL of the activated proteasome was added and mixed. The samples were pre-incubated in the Fluoroskan for 5 minutes at 37° C. Substrate was added and the kinetics of AMC were followed for one hour. All data were collected and plotted as the mean of triplicate data points. The data were normalized to reactions performed in the absence of Salinosporamide A and modeled in Prism as a sigmoidal dose-response, variable slope.

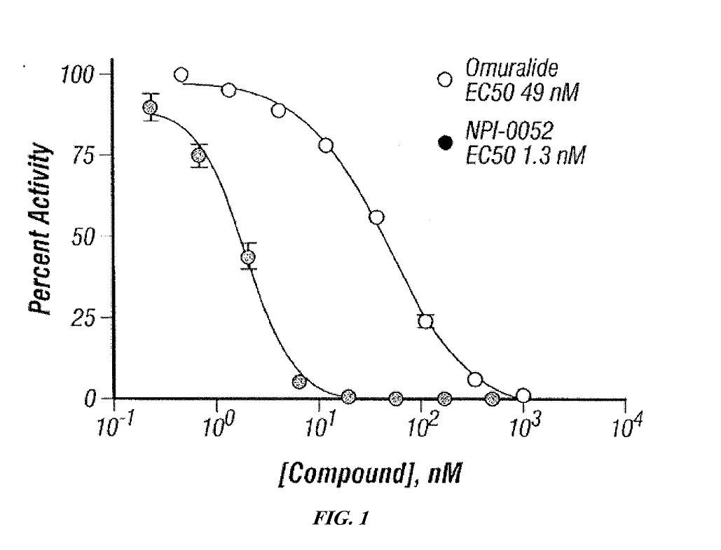

Similar to the results obtained for the in vitro cytotoxicity, Feling, et al., Angew Chem Int Ed Engl 42:355 (2003), the EC₅₀ values in the 20S proteasome assay showed that Salinosporamide A (NPI-0052) was approximately 40-fold more potent than Omuralide, with an average value of 1.3 nM versus 49 nM, respectively (FIG. 1). This experiment was repeated and the average EC₅₀ in the two assays was determined to be 2 nM for Salinosporamide A and 52 nM for Omuralide.

Salinosporamide A is a potent inhibitor of the chymotrypsin-like activity of the proteasome. The EC₅₀ values for cytotoxicity were in the 10-200 nM range suggesting that the ability of Salinosporamide A to induce cell death was due, at least in large part, to proteasome inhibition. The data suggest that Salinosporamide A is a potent small molecule inhibitor of the proteasome.

### Example 34

### Salinosporamide A (II-16) Inhibition of PGPH Activity of Rabbit Muscle 20S Proteasomes

Omuralide can inhibit the PGPH activity (also known as the caspase-like) of the proteasome; therefore, the ability of Salinosporamide A to inhibit the PGPH activity of purified rabbit muscle 20S proteasomes was assessed. A commercially available fluorogenic substrate specific for the PGPH activity was used instead of the chymotrypsin substrate supplied in the proteasome assay kit described above.

Salinosporamide A (II-16) was prepared as a 20 mM solution in DMSO and stored in small aliquots at −80° C. The substrate Z-LLE-AMC was prepared as a 20 mM stock solution in DMSO, stored at −20° C. The source of the proteasomes was the commercially available kit from Calbiochem (Cat. #539158). As with the chymotrypsin substrate, the proteasome can cleave Z-LLE-AMC into Z-LLE and free AMC. The activity can then be determined by measuring the fluorescence of the released AMC (λₑₓ=390 nm and λₑₘ=460 nm). The proteasomes were activated with SDS and held on ice as per manufacturer's recommendation. Salinosporamide A was diluted in DMSO to generate a 400-fold concentrated 8-point dilution series. The series was diluted 20-fold with assay buffer and preincubated with the proteasomes as described for the chymotrypsin-like activity. After addition of substrate, the samples were incubated at 37° C., and release of the fluorescent AMC was monitored in a fluorimeter. All data were collected and plotted as the mean of triplicate points. In these experiments, the EC₅₀ was modeled in Prism as normalized activity, where the amount of AMC released in the absence of Salinosporamide A represents 100% activity. As before, the model chosen was a sigmoidal dose-response, with a variable slope.

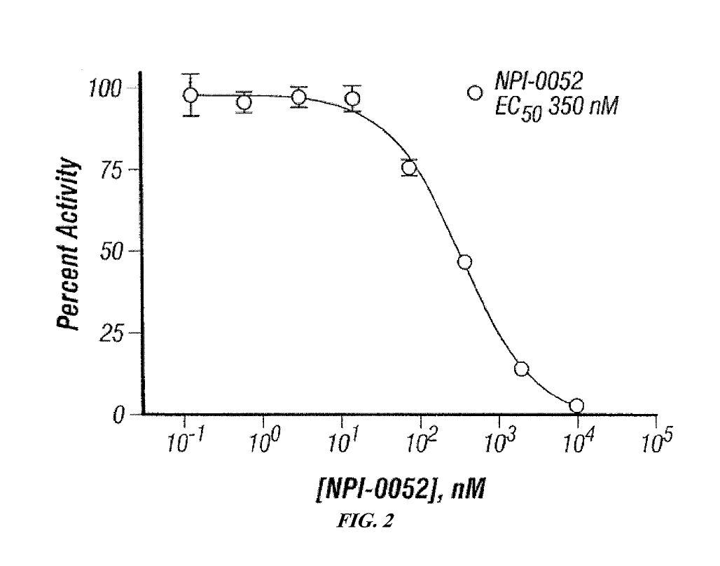

Data revealed that Salinosporamide A (NPI-0052) inhibited the PGPH activity in rabbit muscle 20S proteasomes with an EC₅₀ of 350 nM (FIG. 2). A replicate experiment was performed, which gave a predicted EC₅₀ of 610 nM. These results indicate that Salinosporamide A does block the in vitro PGPH activity of purified rabbit muscle 20S proteasomes, albeit with lower potency than seen towards the chymotrypsin-like activity.

### Example 35

### Inhibition of the Chymotrypsin-Like Activity of Human Erythrocyte 20S Proteasomes

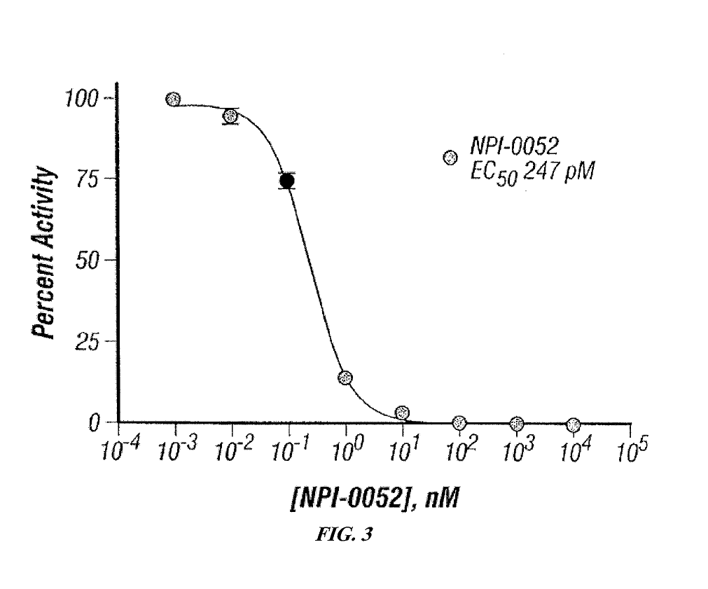

The ability of Salinosporamide A (II-16) to inhibit the chymotrypsin-like activity of human erythrocyte 20S proteasomes was assessed in vitro. The calculated EC₅₀ value is approximately 3 nM (FIG. 3). These data indicate that the inhibitory effect of Salinosporamide A is not limited to rabbit skeletal muscle proteasomes.

Salinosporamide A was prepared as a 20 mM solution in DMSO and stored in small aliquots at −80° C. The substrate, suc-LLVY-AMC, was prepared as a 20 mM solution in DMSO and stored at −20° C. Human erythrocyte 20S proteasomes were obtained from BIOMOL (Cat. #SE-221). The proteasome can cleave suc-LLVY-AMC into suc-LLVY and free AMC and the activity can then be determined by measuring the fluorescence of the released AMC (λex=390 nm and λₑₘ=460 nm). The proteasomes were activated by SDS and stored on ice as with the experiments using rabbit muscle proteasomes. Salinosporamide A was diluted in DMSO to generate a 400-fold concentrated 8-point dilution series. The series was then diluted 20-fold with assay buffer and pre-incubated with proteasomes at 37° C. The reaction was initiated with substrate, and the release of AMC was followed in a Fluoroskan microplate fluorimeter. Data were collected and plotted as the mean of triplicate points. Data were captured kinetically for 3 hours, and indicated that these reactions showed linear kinetics in this time regime. The data were normalized to reactions performed in the absence of Salinosporamide A and modeled in Prism as a sigmoidal dose-response, variable slope.

Replicate experiments performed using human erythrocyte proteasomes from separate lots resulted in a range of EC₅₀ values of approximately 4 nM. These results indicate that the in vitro chymotrypsin-like activity of human erythrocyte 20S proteasomes is sensitive to Salinosporamide A.

Formula II-16 also showed inhibition of the Trypsin-like and Caspase-like activity of human erythrocyte proteasomes. For Trypsin-like the studies showed an EC₅₀ value of about 9 nM, and for Caspase-like an EC₅₀ of about 390 nM. Additional studies of Chymotrypsin-like activity in human erythrocytes resulted in an EC₅₀ of about 250 pM. Furthermore, studies showed that Formula II-16 is specific for the proteasome, showing little or no effect on other proteolytic enzymes. For example, Formula II-16 when tested for inhibition of Chymotrypsin, Cathepsin B and Thrombin, respectively, had EC₅₀ values of 18,000 nM, >200,000 nm, and >200,000 nM, respectively.

### Example 36

### Anti-Tuberculosis Activity

The activity of Salinosporamide A in inhibition of the proteasome of mycobacterium tuberculosis is tested by measuring proteasomal protease activity in cell lysates. To measure proteasomal protease activity, mycobacterium tuberculosis is lysed by agitation with zirconia silica beads. The soluble fraction is filtered through a 0.45 micron filter. Aliquots of the filtrate (120 μg protein) are incubated with succinyl-leu-leu-val-tyr-α-methylcoumarin in the presence of 0.05% SDS and fluorescence is monitored. These conditions report activity of the mycobacterial proteasome. Knipfer, et al., Mol Microbiol 25: 375 (1997). The 50% inhibitory concentration is determined by applying the Hill equation to data from 2 experiments, each in triplicate.

Salinosporamide A is assayed in liquid culture to test its ability to inhibit recovery of wild-type Mycobacterium tuberculosis from nitrite-mediated injury. Mycobacterium tuberculosis is incubated with either no compound, or Salinosporamide A in 7H9-ADNaCl at pH 5.5 with or without 3 mM nitrite. Bacteria is subcultured into fresh 7H9-ADNaCl at pH 6.6. Outgrowth of surviving bacteria is measured by optical density (A₅₈₀). Outgrowth is measure 6 days after subculture of Mycobacterium tuberculosis that is incubated in medium at pH 5.5 without nitrite. Outgrowth is measured 15 days after subculture of Mycobacterium tuberculosis that is incubated in medium at pH 5.5 with nitrite. Following the exposure to nitrite, a longer period of outgrowth of surviving bacteria is necessary before absorbance becomes detectable.

In survival assays Salinosporamide A is evaluated based on the growth of Mycobacterium tuberculosis on agar plates. Salinosporamide A is able to augment the antimycobacterial effect of nitrite when the inhibitors and nitrite are removed simultaneously by plating bacteria on agar after 6 days of exposure. Salinosporamide A is able to augment the antimycobacterial effect of nitrite, if present, after nitrite mediated injury. Salinosporamide A is able to enhance the antimycobacterial effect when added along with nitrite at day 0 and when added only after the subculture on day 6, plating on day 10. Salinosporamide A is able to increase the antimycobacterial activity of nitrite when Mycobacterium tuberculosis is given time to recover during a 4-day period of subculture at pH 6.5 before being plated. Salinosporamide A is also effective if added at the time of subculture.

### Example 37

### Treatment of Tuberculosis

A human patient diagnosed with tuberculosis is administered a compound described herein. After administration, the symptoms of tuberculosis are ameliorated. In one experiment, the patient is cured after continued administration of a compound described herein.

### Example 38

### In Vivo Biology Maximum Tolerated Dose (MTD) Determination

In vivo studies were designed to determine the MTD of Salinosporamide A when administered intravenously to female BALB/c mice.

BALB/c mice were weighed and various Salinosporamide A concentrations (ranging from 0.01 mg/kg to 0.5 mg/kg) were administered intravenously as a single dose (qdx1) or daily for five consecutive days (qdx5). Animals were observed daily for clinical signs and were weighed individually twice weekly until the end of the experiment (maximum of 14 days after the last day of dosing). Results are shown in Table 1 and indicate that a single intravenous Salinosporamide A dose of up to 0.25 mg/kg was tolerated. When administered daily for five consecutive days, concentrations of Salinosporamide A up to 0.1 mg/kg were well tolerated. No behavioral changes were noted during the course of the experiment.

TABLE 1
MTD DETERMINATION OF SALINOSPORAMIDE
A IN FEMALE BALB/C MICE

| | Dose | | | Days of |
|---|---|---|---|---|
| Group | (mg/kg) | Route/Schedule | Deaths/Total | Death |
| 1 | 0.5 | i.v.; qdx1 | 3/3 | 3, 3, 4 |
| 2 | 0.25 | i.v.; qdx1 | 0/3 | |
| 3 | 0.1 | i.v.; qdx1 | 0/3 | |
| 4 | 0.05 | i.v.; qdx1 | 0/3 | |
| 5 | 0.01 | i.v.; qdx1 | 0/3 | |
| 6 | 0 | i.v.; qdx1 | 0/3 | |
| 7 | 0.5 | i.v.; qdx5 | 3/3 | 4, 6, 7 |
| 8 | 0.25 | i.v.; qdx5 | 3/3 | 4, 5, 5 |
| 9 | 0.1 | i.v.; qdx5 | 0/3 | |
| 10 | 0.05 | i.v.; qdx5 | 0/3 | |
| 11 | 0.01 | i.v.; qdx5 | 0/3 | |
| 12 | 0 | i.v.; qdx5 | 0/3 | |

### Example 39

### Preliminary Assessment of Salinosporamide A Absorption, Distribution, Metabolism and Elimination (ADME) Characteristics

Studies to initiate the evaluation of the ADME properties of Salinosporamide A were performed. These studies consisted of solubility assessment, LogD^{7.4} determination and a preliminary screen to detect cytochrome P450 enzyme inhibition. Results from these studies showed an estimated solubility of Salinosporamide A in PBS (pH 7.4) of 9.6 μM (3 μg/ml) and a LogD^{7.4} value of 2.4. This LogD^{7.4} value is within the accepted limits compatible with drug development (LogD^{7.4}<5.0) and suggests oral availability. Results from the preliminary P450 inhibition screen showed that Salinosporamide A, when tested at 10 μM, showed no or low inhibition of all P450 isoforms: CYP1A2, CYP2C9 and CYP3A4 were inhibited by 3%, 6% and 6% respectively, while CYP2D6 and CYP2C19 were inhibited by 19% and 22% respectively.

### Example 40

### Salinosporamide A and its Effects In Vivo on Whole Blood Proteasome Activity

Salinosporamide A was previously demonstrated to be a potent and specific inhibitor of the proteasome in vitro, with an IC₅₀ of 2 nM towards the chymotrypsin-like activity of purified 20S proteasomes. To monitor the activity of Salinosporamide A in vivo, a rapid and reproducible assay (adapted from Lightcap et al. 2000) was developed to assess the proteosome activity in whole blood.

In brief, frozen whole blood samples were thawed on ice for one hour, and resuspended in 700 μL of ice cold 5 mM EDTA, pH 8.0 in order to lyse the cells by hypotonic shock. This represents approximately 2-3 times the volume of the packed whole blood cells. Lysis was allowed to proceed for one hour, and the cellular debris was removed by centrifugation at 14,000×g for 10 minutes. The supernatant (Packed Whole Blood Lysate, PWBL) was transferred to a fresh tube, and the pellet discarded. Protein concentration of the PWBL was determined by BCA assay (Pierce) using BSA as a standard. Approximately 80% of the samples had a total protein concentration between 800 and 1200 μg/mL.

Proteasome activity was determined by measuring the hydrolysis of a fluorogenic substrate specific for the chymotrypsin-like activity of proteasomes (suc-LLVY-AMC, Bachem Cat. I-1395). Control experiments indicated that >98% of the hydrolysis of this peptide in these extracts is mediated by the proteasome. Assays were set up by mixing 5 μL of a PWBL from an animal with 185 μL of assay buffer (20 mM HEPES, 0.5 mM EDTA, 0.05% Triton X-100, 0.05% SDS, pH 7.3) in Costar 3904 plates. Titration experiments revealed there is a linear relationship between protein concentration and hydrolysis rate if the protein concentration in the assay is between 200 and 1000 μg. The reactions were initiated by the addition of 10 μL of 0.4 mM suc-LLVY-AMC (prepared by diluting a 10 mM solution of the peptide in DMSO 1:25 with assay buffer), and incubated in a fluorometer (Labsystems Fluoroskan) at 37° C. Hydrolysis of the substrate results in the release of free AMC, which was measured fluorometrically by using λₑₓ=390 nm and λₑₘ=460 nm. The rate of hydrolysis in this system is linear for at least one hour. The hydrolysis rate of each sample is then normalized to relative fluorescent units per milligram of protein (RFU/mg).

To explore the in vivo activity of Salinosporamide A, male Swiss-Webster mice (5 per group, 20-25 g in weight) were treated with various concentrations of Salinosporamide A. Salinosporamide A was administered intravenously and given its LogD^{7.4} value of 2.4, suggestive of oral availability, Salinosporamide A was also administered orally. Salinosporamide A dosing solutions were generated immediately prior to administration by dilution of Salinosporamide A stock solutions (100% DMSO) using 10% solutol yielding a final concentration of 2% DMSO. The vehicle control consisted of 2% DMSO in 10% solutol. One group of animals was not dosed with either vehicle or Salinosporamide A in order to establish a baseline for proteasome activity. Salinosporamide A or vehicle was administered at 10 mL/kg and ninety minutes after administration the animals were anesthetized and blood withdrawn by cardiac puncture. Packed whole blood cells were collected by centrifugation, washed with PBS, and re-centrifuged. All samples were stored at −80° C. prior to the evaluation of the proteasome activity.

In order to be certain that the hydrolysis of the substrate observed in these experiments was due solely to the activity of the proteasome, dose response experiments on the extracts were performed using the highly specific proteasomal inhibitor Epoxomicin. PWBL lysates were diluted 1:40 in assay buffer, and 180 μL were added to Costar 3904 plates. Epoxomicin (Calbochem Cat. 324800) was serially diluted in DMSO to generate an eight point dose response curve, diluted 1:50 in assay buffer, and 10 μL added to the diluted PWBL in triplicate. The samples were preincubated for 5 minutes at 37° C., and the reactions initiated with substrate as above. The dose response curves were analyzed in Prism, using a sigmoidal dose response with variable slope as a model.

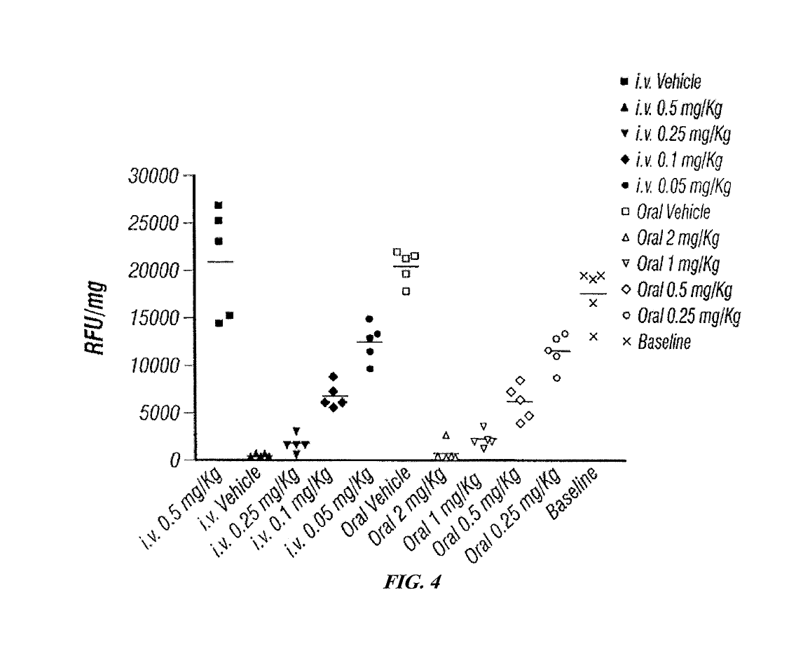

FIG. 4 is a scatter plot displaying the normalized proteasome activity in PWBL's derived from the individual mice (5 mice per group). In each group, the horizontal bar represents the mean normalized activity. These data show that Salinosporamide A causes a profound decrease in proteasomal activity in PWBL, and that this inhibition is dose dependent. In addition, these data indicate that Salinosporamide A is active upon oral administration.

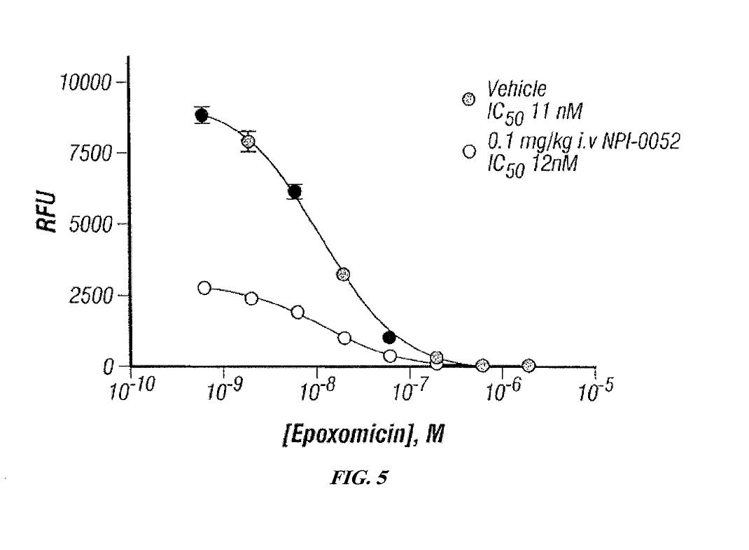

The specificity of the assay was shown by examining the effect of a known proteasome inhibitor, Epoxomicin, on hydrolysis of the peptide substrate. Epoxomicin is a peptide epoxide that has been shown to highly specific for the proteasome, with no inhibitory activity towards any other known protease (Meng et al., 1999). Lysates from a vehicle control and also from animals treated intravenous (i.v.) with 0.1 mg/kg Salinosporamide A were incubated with varying concentration of Epoxomicin, and IC₅₀ values were determined. Palayoor et al., Oncogene 18:7389-94 (1999). As shown in FIG. 5, Epoxomicin caused a dose dependent inhibition in the hydrolysis of the proteasome substrate. The IC₅₀ obtained in these experiments matches well with the 10 nM value observed using purified 20S proteasomes in vitro (not shown). These data also indicate that the remaining activity towards this substrate in these lysates prepared from animals treated with 0.1 mg/kg Salinosporamide A is due to the proteasome, and not some other protease. The residual activity seen in extracts treated with high doses of Epoxomicin is less than 2% of the total signal, indicating that over 98% of the activity observed with suc-LLVY-AMC as a substrate is due solely to the activity of the proteasomes present in the PWBL.

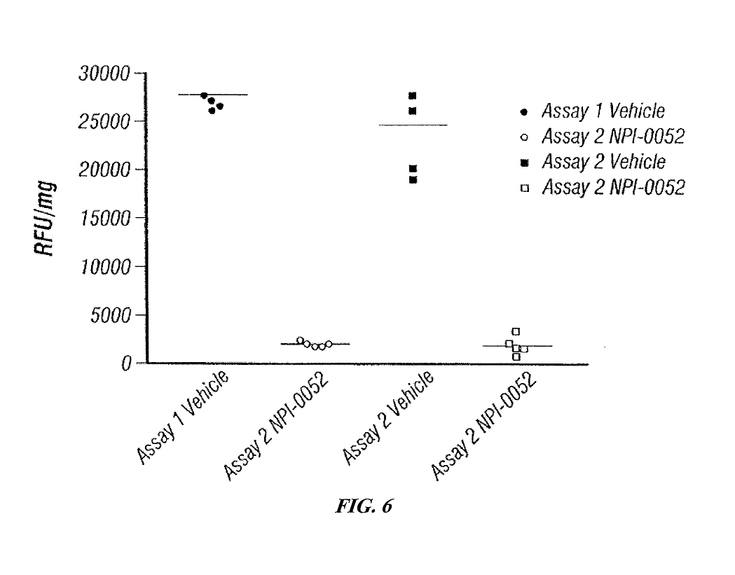

Comparison of intra-run variation in baseline activity and the ability of Salinosporamide A to inhibit proteasomal activity was also assessed. In FIG. 6, the results of separate assays run several weeks apart are shown. Qureshi, et al., J. Immunol. 171(3):1515-25 (2003). For clarity, only the vehicle control and matching dose results are shown. While there was some variation in the proteasomal activity in PWBL derived from individual animals in the control groups, the overall mean was very similar between the two groups. The animals treated with Salinosporamide A (0.1 mg/kg i.v.) also show very similar residual activity and average inhibition.

### Example 41

### Structure Activity Relationships

While not being bound by any particular theory certain compounds disclosed herein have been shown to have beneficial activity. With regard to Formula II, compounds having a halogenated substituent at R₁ are preferred. Most preferred are n-halogenated ethyl at R₁.

Also, most preferred are compounds with a hydroxy group at E₅ and the attached carbon is in an S conformation (compounds having the stereochemistry of compound II-18, for example). Oxidation from a hydroxyl group to a ketone is less preferred.

In one preferred embodiment, the preferred substituent at R₄ is cyclohexene. In another preferred embodiment, the cyclohexene is oxidized to an epoxide. Less preferred are compounds with hydrogenation of the double bond of the cyclohexene substituent.

Furthermore in some embodiments, preferably, R₃ is methyl, with ethyl being less preferred.

### Example 42

### Formulation to be Administered Orally or the Like

A mixture obtained by thoroughly blending 1 g of a compound obtained and purified by the method of the embodiment, 98 g of lactose and 1 g of hydroxypropyl cellulose is formed into granules by any conventional method. The granules are thoroughly dried and sifted to obtain a granule preparation suitable for packaging in bottles or by heat sealing. The resultant granule preparations are orally administered at between approximately 100 ml/day to approximately 1000 ml/day, depending on the symptoms, as deemed appropriate by those of ordinary skill in the art of treating cancerous tumors in humans.

### Example 43

### Antimicrobial Assays

Minimum inhibitory concentrations (MICs) are determined according to the National Committee for Clinical Laboratory Standards (NCCLS) susceptibility test guideline M7-A5 (Ferraro, M. 2001 Methods for Dilution Antimicrobial Susceptibility Tests for Bacteria that Grow Aerobically; Approved Standard (NCCLS). National Committee for Clinical Laboratory Standards (NCCLS), Villanova, which is incorporated herein by reference in its entirety). The compound of formula II-16 is tested in an appropriate solvent for the antimicrobial assay. Antimicrobial data for the compounds of formula II-16 is determined in a variety of infectious diseases.

The examples described above are set forth solely to assist in the understanding of the embodiments. Thus, those skilled in the art will appreciate that the methods may provide derivatives of compounds.

One skilled in the art would readily appreciate that the present invention is well adapted to carry out the objects and obtain the ends and advantages mentioned, as well as those inherent therein. The methods and procedures described herein are presently representative of preferred embodiments and are exemplary and are not intended as limitations on the scope of the invention. Changes therein and other uses will occur to those skilled in the art which are encompassed within the spirit of the invention.

It will be readily apparent to one skilled in the art that varying substitutions and modifications can be made to the embodiments disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably can be practiced in the absence of any element or elements, limitation or limitations which is not specifically disclosed herein. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention. Thus, it should be understood that although the present invention has been specifically disclosed by preferred embodiments and optional features, modification and variation of the concepts herein disclosed can be resorted to by those skilled in the art, and that such modifications and variations are considered to be falling within the scope of the embodiments of the invention.

What is claimed is:

1. A method of treating an infectious disease comprising administering to an animal a compound having the structure of any one of Formulas I and II, or a pharmaceutically acceptable salt or pro-drug thereof: 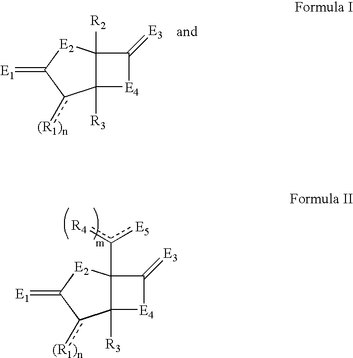
wherein:
the dashed lines represent a single or a double bond;
each R₁ is separately a hydrogen, a halogen, a cyano, a nitro, an azido, a hydroxy, or a thiocyano, or selected from the group consisting of optionally substituted: C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl;
n is 1 or 2, where if n is 2, then each R₁ can be the same or different;
m is 1 or 2, where if m is 2, then each R₄ can be the same or different;
R₂ is a hydrogen, a halogen, a cyano, a nitro, an azido, a hydroxy, or a thiocyano, or selected from the group consisting of optionally substituted: C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl;
R₃ is a halogen or selected from the group consisting of optionally substituted C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, aminocarbonyl, aminocarbonyloxy, nitro, azido, phenyl, cycloalkylacyl, hydroxy, alkylthio, arylthio, oxysulfonyl, carboxy, cyano, and halogenated alkyl including polyhalogenated alkyl;
each of E₁, E₃, E₄ and E₅ is an optionally substituted heteroatom;
E₂ is an optionally substituted heteroatom or —CH₂— group;
each R₄ is separately a halogen, a cyano, a nitro, an azido, or a thiocyano, or selected from the group consisting of optionally substituted C₁-C₂₄ alkyl, C₂-C₂₄ alkenyl, C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxycarbonyl, alkoxycarbonylacyl, amino, hydroxy, aminocarbonyl, aminocarbonyloxy, phenyl, cycloalkylacyl, alkylthio, arylthio, oxysulfonyl, carboxy, thio, sulfoxide, sulfone, sulfonate esters, boronic acids and esters, and halogenated alkyl including polyhalogenated alkyl; and
wherein the infectious disease is selected from the group consisting of Bacteremia, Botulism, Brucellosis, Clostridium Difficile, Campylobacter Infection, Cat Scratch Disease, Chancroid, Chlamydia, Cholera, Clostridium Perfringens, Bacterial Conjunctivitis, Diphtheria, E. Coli Infections, Ehrlichiosis, Epididymitis, Gardnerella, Gas Gangrene, Gonorrhea, Helicobacter Pylori, Haemophilus, Influenzae B, Impetigo, Intertrigo, Leprosy, Listeriosis, Lyme Disease, Methicillin Resistant Staphylococcus Aureus, Orchitis, Osteomyelitis, Otitis, Media Pertussis, Plague, Pneumonia, Prostatitis Pyelonephritis, Q Fever, Rocky Mountain Spotted Fever, Salmonellosis, Scarlet Fever, Sepsis, Shigellosis, Staphylococcal Infections, Streptococcal Infections, Syphilis, Tetanus, Toxic Shock Syndrome, Trachoma, Traveller's Diarrhea, Tuberculosis, Tularemia, Typhoid Fever, Typhus Fever, Urinary Tract Infections, Bacterial Vaginosis, Pertussis, Yersiniosis, malaria, African trypanosomiasis, candidiasis, histoplasmosis, blastomycosis, coccidioidomycosis, aspergillisis, and mucormycosis.

2. The method of claim 1, wherein the compound is Salinosporamide A:

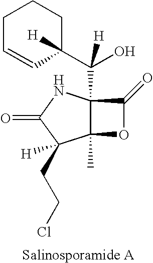

3. The method of claim 1, wherein the infectious disease is caused by a bacterial infection.

4. The method of claim 3, wherein the compound is Salinosporamide A:

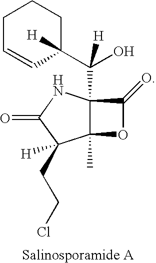

5. The method of claim 3, wherein the bacterial infectious disease is Tuberculosis.

6. The method of claim 5, wherein the compound is Salinosporamide A:

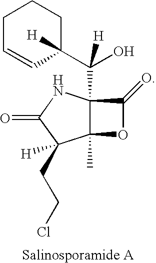

7. The method of claim 5, wherein the bacteria causing Tuberculosis is selected from the group consisting of Mycobacterium bovis, Mycobacterium africanum and Mycobacterium microti.

8. The method of claim 7, wherein the compound is Salinosporamide A:

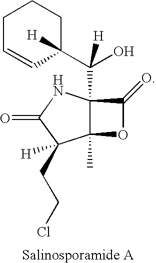

9. The method of claim 5, wherein the bacteria causing Tuberculosis is Mycobacterium tuberculosis.

10. The method of claim 9, wherein the compound is Salinosporamide A:

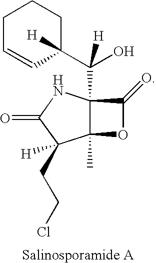

11. The method of any one of the claims 1, further comprising co-administering one or more anti-infective agent(s).

12. The method of claim 11, wherein the compound is Salinosporamide A:

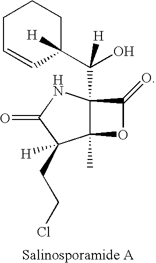

13. The method of the claim 11, wherein the anti-infective agent(s) is selected from the group consisting of isoniazid, rifampin, ethambutol, pyrazinamide, rifater, streptomycin, rifapentine and epoxomicin.

14. The method of claim 13, wherein the compound is Salinosporamide A:

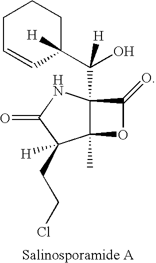

15. The method of any one of the claims 1, wherein the animal is a human.

16. The method of claim 15, wherein the compound is Salinosporamide A: [chemical structure]
